(19) United States
(12) Patent Application Publication
(10) Pub. No.: US 2026/0007134 A1
(43) Pub. Date: Jan. 8, 2026

(54) BIOLOGICAL CONTROL OF SOFT ROT AND BIOFILMS BY MICROBIAL PREDATORS ENCAPSULATED IN CARRIERS

(71) Applicants: YISSUM RESEARCH DEVELOPMENT COMPANY OF THE HEBREW UNIVERSITY OF JERUSALEM LTD., Jerusalem (IL); THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Amos NUSSINOVITCH, Rehovot (IL); Gal SASON, Rehovot (IL); Edouard JURKEVITCH, Ness Ziona (IL); Iris YEDIDIA, Rehovot (IL)

(21) Appl. No.: 18/992,397

(22) PCT Filed: Jul. 10, 2023
(86) PCT No.: PCT/IL2023/050716

§ 371 Date: Jan. 8, 2025

Related U.S. Application Data
(60) Provisional application No. 63/359,905, filed on Jul. 11, 2022.
(60) Provisional application No. 63/499,998, filed on May 4, 2023.

(51) Int. Cl. A01N 63/20; A01N 25/10; A01P 1/00

(52) U.S. Cl. CPC A01N 63/20; A01N 25/10; A01P 1/00

(57) ABSTRACT

The present invention provides systems for differential delivery of viable BALOs. The systems comprise a carrier comprising a polysaccharide hydrocolloid, and BALOs immobilized within the carrier, wherein the BALOs are present in a physiological state selected from bdelloplasts, bdellocysts, and APs, which are present at a cell density of at least about 1.0×10⁸ PFU/(g carriers), wherein the polysaccharide hydrocolloid is in a wet or dried-gel form, and wherein the carrier preserves viability and bacteriolytic efficiency of the immobilized BALOs. Further provided are methods for the preparation of the systems for differential delivery of viable BALOs, and methods for use thereof, such as for treatment of phytopathogenic bacterial diseases in plants or crop.

### FIELD OF THE INVENTION

The present invention is directed to hydrocolloid-based compositions for encapsulation and delivery of viable Bdellovibrio-and-like organisms (BALOs) and uses thereof.

### BACKGROUND OF THE INVENTION

Biofilms are matrix-enclosed microbial aggregates that adhere to a biological or non-biological surface. The complex microbial community of a biofilm is highly resistant to antibiotics and sanitizers and confers persistent survival that is a challenge to overcome. Biofilm formation is a significant problem in various industries and can lead to substantial economic and health problems. Many outbreaks of pathogens have been attributed to biofilms. At water and sewage treatment facilities and water distribution systems, biofilms (biofouling) cause metal corrosion, increase the risk of contamination, decrease the quality of water, and reduce the efficacy of heat exchange.

The Pectobacterium and Dickeya pectinolytic bacteria belonging to the Pectobacteriaceae family are phytopathogens responsible for several macerating diseases (soft rot, black leg and aerial stem rot) on a wide range of crops and ornamental plants in a variety of climates. These diseases are accountable for paramount economic damages with estimated global crop losses of at least 30%. As of today, there are no effective physical and chemical controls against this group of bacteria and most of the practices to protect crops are based on sanitation, nutrition, and growth management.

However, recent findings show that Bdellovibrio and like organisms (BALOs) are promising biocontrol agents against the soft rot Pectobacteriaceae (SRP). BALOs are a group of small, highly motile gram-negative bacteria that predate upon other gram-negative bacteria and are found in many terrestrial and aquatic environments (Rotem et al., 2014). These bacteria interact with their prey as highly motile attack phase (AP) cells, by attaching to the outer membrane and consuming the prey extracellularly (epibiotic predation), or by penetrating their periplasm (periplasmic predation). Yet, most BALO isolates, including Bdellovibrio bacteriovorus, the most studied of the BALOs, are periplasmic predators. During prey invasion, the predator alters the prey cell wall, preventing surinfection, and transforming it into a shielded feeding niche called a bdelloplast. Within it, the BALO cell grows to finally yield progeny AP cells that escape the prey remains (Rotem et al., 2014). The BALOs' prey range varies between strains, some exhibiting high specificity to a certain prey, while others are generalist-like, using a wide range of prey. Community and single cell analyses reveal complex predatory interactions. Genetic resistance to BALOs is not easily acquired, yet prey populations can exhibit plastic phenotypic resistance (Shemesh et al., 2004; Duncan et al., 2018; Aharon et al., 2021).

These properties give BALOs the potential to be an alternative source of antibiotics (Sockett, 2009). However, application of AP cells or bdelloplasts, while showing great potential to protect against pathogens of humans and other animals as well as against plant pathogens, is not common (Duncan et al., 2018). It requires freshly grown BALO cultures which imposes several limitations in terms of shelf-life, transport, and handling (Mansfield et al., 2012). Therefore, practical implementation requires suitable delivery vehicles.

BALO encapsulation in gelatin microparticles was reported for use in aquaculture systems (Mansfield et al., 2012). Encapsulated bdellovibrio powder has been proposed as a potential bio-disinfectant against whiteleg shrimp-pathogenic vibrios (Cao et al., 2019). Polyhydroxyalkanoate carriers were developed for BALO delivery in the industrial production of biological plastics, for example B. bacteriovorus HD100 encapsulation (González et al., 2020)

Since biocontrol includes the use of sensitive living organisms, a suitable encapsulation system provides several advantages over free cell inoculation. Encapsulation is a process of forming a continuous layer entrapping cells and/or compounds within a matrix core. Encapsulation within a matrix protects the living cells from biotic and abiotic stress factors by providing a beneficial microenvironment. Proper matrix formulation has the potential to improve the application of microbial biological control agents by extending shelf life, decreasing the number of applications, and reducing doses as it can provide sufficient cell density while keeping cell performance, and simplify handling (Vemmer, 2013). For such preparations, water-soluble polymeric materials (hydrocolloids) extracted from natural sources have been widely used to produce gel-based carriers for the encapsulation of microorganisms in the food, biotechnology, and agriculture industries.

Hydrocolloid carriers encapsulating microorganisms can be tailored to specific needs. For example, pectin has been widely used as a delivery vehicle for colon-specific oral drugs and, to a lesser extent, probiotics (low-methoxyl-pectin and rice-bran) (Chotiko et al., 2016; Jung et al., 2013). Few reports studied the controlled-release of kasugamycin (antibiotic) conjugated with pectin against phytopathogens with pectinase activity (Fan et al., 2017; Liu et al., 2015).

Recently, Sason et al. disclosed the biological control of soft rot in potato by BALOs entrapped within K-carrageenan carriers (Applied Microbiology and Biotechnology (2023) 107:81-96), and the self-demise of soft rot bacteria by activation of BALOs by pectin-based carriers (Microbial Biotechnology (2023) 16:1561-1576).

There remains an unmet need for encapsulation and delivery systems for viable BALOs to be used, inter alia, in agricultural and industrial applications to prevent bacterial soft rot infections in plant tissues and reduce biofilm formation. The encapsulation and delivery systems should provide long shelf life, as well as offer controllable release times and profiles of the encapsulated viable BALOs that can be tailored to specific applications and types of bacteria.

### SUMMARY OF THE INVENTION

The present invention provides systems for differential delivery of viable BALOs, methods for the preparation thereof, and methods for treating plants and crop by using said systems. The systems for differential delivery of viable BALOs are based on encapsulation in a polysaccharide hydrocolloid. The polysaccharide hydrocolloids with the encapsulated BALOs can be in a dried gel form, thereby offering long shelf-life and high stability of the viable BALOs.

The present invention is based in part on an unexpected finding that polysaccharide hydrocolloids can provide an efficient immobilization and delivery system for BALOs such that the carriers based on said polysaccharide hydrocolloids can entrap BALOs at a cell density of at least about 1.0×10⁸ PFU/(g carriers). The entrapped BALOs within the delivery system of the invention are present in a physiological state of bdelloplasts, bdellocysts and/or attack phase (AP) cells. According to some embodiments, the BALOs are present in a physiological state of bdelloplasts and AP cells.

It has surprisingly been discovered that the survival rates of the BALOs after drying the hydrocolloid-based delivery system were higher for bdelloplasts as compared to attack phase BALO cells. Additionally, different polysaccharide hydrocolloids were used to provide time-dependent, controllable release of the encapsulated BALOs. As exemplified hereinbelow, carrageenans were found to enable long-term entrapment of BALOs and fast delivery thereof upon swelling and dissolution of the dried hydrocolloid beads following contact with water. As such, the carrageenan-based carriers provide an efficient fast delivery system of BALOs for water-related applications, such as crop protection against soft rot disease and biofilm disruption. In contrast, pectin-based carriers ensured a long-term entrapment of the BALOs within the carrier and allowed targeted delivery at a certain location and at a sustained release manner. Without wishing to be bound by any theory or mechanism of action, it is contemplated that the degradation of the pectin-based delivery system is caused by pectinolytic enzymes secreted from the prey itself, i.e., soft rot Enterobacteria (SREs), the latter are triggered by the pectin of the delivery system, and as a result of the degradation of the delivery system, the predator is released in an “attract and kill” state. In this manner, the use of BALOs can be localized, minimizing potential damage by the biocontrol agent to beneficial microorganisms. The present invention therefore provides a universal system for differential delivery of viable, bacteriolytic BALOs, which system can be tailored to provide the desired delivery profile of the viable BALOs.

It is now further disclosed that while BALOs viability in liquid suspensions at 25° C. decreased sharply, resulting in no viable cells after two months of storage, the dried delivery systems of the present invention comprising BALOs immobilized within carrageenan-based carriers preserved viable BALOs at a cell density of at least 1.0×10¹⁰ PFU/(g carriers) after one year of storage at 25° C. It is also disclosed that while liquid suspensions of BALOs lost viability within six months of storage at 4° C., the dried delivery systems of the invention which comprise BALOs immobilized within carrageenan-based carriers preserved viable BALOs at a cell density of at least 1.0×10¹⁰ PFU/(g carriers) after one year of storage at 4° C., and the dried delivery systems of the invention which comprise BALOs immobilized within pectin-based carriers preserved viable BALOs at a cell density of at least 1.0×10⁸ PFU/(g carriers) after one year of storage at the same temperature. Thus, the delivery systems of the present invention are highly useful for long-term storage of viable, bacteriolytic BALOs.

It is further disclosed that the dried delivery systems of the present invention which comprise BALOs immobilized within carrageenan-based carriers were found to be highly effective in reducing bacterial soft rot infections in potato tubers under controlled conditions as well as under natural environmental conditions.

Thus, the delivery systems of the present invention, and in particular the dried form of these systems, which comprise BALOs immobilized/entrapped within polysaccharide hydrocolloids-based carriers, are highly effective, non-toxic, biodegradable biocontrol agents which enable achieving cell density of the viable bacterial predators of at least 1.0×10⁸, or at least 1.0×10⁹, or even at least 1.0×10¹⁰ PFU/(g carriers); provide controlled and localized release of BALOs; and preserve BALOs viability and bacteriolytic efficiency during months of storage. The delivery systems of the present invention are therefore useful in a variety of applications, including in agriculture, e.g., reducing macerating diseases on crops.

According to one aspect, the present invention provides a system for differential delivery of viable Bdellovibrio-and-like organisms (BALOs) comprising:
a carrier comprising a polysaccharide hydrocolloid, and
BALOs immobilized within the carrier, wherein the BALOs are in a physiological state selected from the group consisting of bdelloplasts, bdellocysts and attack phase cells (APs), which are present at a cell density of at least about 1.0×10⁸ PFU/(g carriers),
wherein the polysaccharide hydrocolloid is in a wet or dried-gel form, and wherein the carrier preserves viability and bacteriolytic efficiency of the immobilized BALOs.

According to some embodiments, the BALOs comprise or consist of Bdellovibrio baculovirus. According to additional embodiments, Bdellovibrio baculovirus are selected from the group consisting of Bdellovibrio baculovirus HD100, Bdellovibrio baculovirus 109 J, and Bdellovibrio baculovirus ΔmerRNA. Each possibility represents a separate embodiment of the invention. According to an exemplary embodiment, Bdellovibrio baculovirus comprise or consist of Bdellovibrio baculovirus HD100. According to a certain embodiment, the BALOs comprise or consist of bdelloplasts and APs. According to another embodiment, the BALOs comprise or consist of bdelloplasts.

According to further embodiments, the carrier is present as a granule, a bead, a capsule, a particle, a powder, and any combination thereof. Each possibility represents a separate embodiment of the invention. According to yet further embodiments, the granule, bead, capsule, or particle has an average Feret diameter ranging from about 0.01 mm to about 10 mm. According to additional embodiments, the granule, bead, capsule, or particle has an average Feret diameter ranging from about 0.1 mm to about 5 mm, or from about 2 mm to about 4 mm.

According to still further embodiments, the polysaccharide hydrocolloid is in the dried-gel form and the system is a dry system. According to one exemplary embodiment, the dry system is present as a powder.

According to some embodiments, the polysaccharide hydrocolloid is selected from the group consisting of κ-carrageenan, pectin, alginate, agar, gellan, carboxymethyl cellulose (CMC), furecellaran, xanthan gum, locust bean gum (LBG), konjac-mannan, chitosan, pullulan, curdlan, and derivatives and combinations thereof. Each possibility represents a separate embodiment of the invention. According to an exemplary embodiment, the polysaccharide hydrocolloid is κ-carrageenan. According to additional exemplary embodiments, pectin is low methoxyl pectin (LMP).

According to further embodiments, the polysaccharide hydrocolloid is present in the system in a weight percent ranging from about 2% to about 25%, out of the total weight of the dry system.

According to some embodiments, the polysaccharide hydrocolloid is crosslinked by a crosslinking agent and/or a gelation-inducing agent. According to additional embodiments, the crosslinking agent comprises a metal cation selected from the group consisting of a potassium ion, calcium ion, barium ion, magnesium ion, aluminum ion, zinc ion, lead ion, iron ion, strontium ion, copper ion, cadmium ion, nickel ion, cobalt ion, and combinations thereof. Each possibility represents a separate embodiment of the invention. According to further embodiments, the metal cation is present in the system in a weight percent ranging from about 0.1% to about 10%, out of the total weight of the dry system.

According to some embodiments, the carrier further comprises a protein hydrocolloid and/or a protein. According to further embodiments, the protein hydrocolloid or protein are selected from the group consisting of gelatin, collagen, collagen hydrolysate, albumin, and any combination thereof. Each possibility represents a separate embodiment of the invention.

According to yet further embodiments, the protein hydrocolloid is gelatin present in the system in a weight percent ranging from about 20% to about 85%, out of the total weight of the dry system.

According to some embodiments, the carrier further comprises a water-absorbing agent. According to further embodiments, the water absorbing agent is selected from the group consisting of starch, polyvinylpyrrolidone (PVP), and derivatives and combinations thereof. Each possibility represents a separate embodiment of the invention.

According to further embodiments, the water-absorbing agent is present in the system in a weight percent ranging from about 25% to about 85%, out of the total weight of the dry system.

According to some embodiments, the carrier further comprises at least one osmoprotectant.

According to further embodiments, the at least one osmoprotectant is selected from the group consisting of trehalose, inositol, glycerol, arabitol, betaine, an amino acid, and combinations thereof. Each possibility represents a separate embodiment of the invention.

According to yet further embodiments, the at least one osmoprotectant is present in the system in a weight percent ranging from about 20% to about 85%, out of the total weight of the dry system.

According to some embodiments, the system comprises from about 0.1% (w/w) to about 10% (w/w) water out of the total weight of the dry system.

According to some embodiments, the differential delivery of the BALOs comprises a fast release or a suspended release of said BALOs from the carrier.

According to some embodiments, the carrier suitable for the fast release of the BALOs comprises κ-carrageenan and potassium cations. According to further embodiments, the carrier further comprises trehalose. According to exemplary embodiments, k-carrageenan is present in the system in a weight percent ranging from about 5% to about 25%, potassium cations are present in a weight percent ranging from about 0.5% to about 10%, and trehalose is present in a weight percent ranging from about 25% to about 85%, out of the total weight of the dry system. According to a certain embodiment, k-carrageenan is present in the system in a weight percent ranging from about 15% to about 25%, potassium cations are present in a weight percent ranging from about 0.5% to about 10%, and trehalose is present in a weight percent ranging from about 70% to about 80%, out of the total weight of the dry system.

According to some embodiments, the carrier suitable for the suspended release of the BALOs comprises low methoxyl pectin (LMP) and calcium ions. According to additional embodiments, the LMP has a degree of esterification (DE) that ranges between about 5% and about 30%, alternatively between about 5% and about 10%, and preferably of about 7%; and a degree of amidation (DA) that ranges between about 0% and about 25%, alternatively between about 0% and about 5%, and preferably the LMP is non-amidated. Each possibility represents a separate embodiment of the invention.

According to further embodiments, the carrier suitable for the suspended release of the BALOs further comprises gelatin or a water-absorbing agent. According to certain embodiments, the LMP is present in the system in a weight percent ranging from about 2% to about 15% and gelatin is present in a weight percent ranging from about 20% to about 85%, out of the total weight of the dry system.

According to further embodiments, the carrier suitable for the suspended release of the BALOs further comprises trehalose. According to yet further embodiments, the LMP is present in the system in a weight percent ranging from about 2% to about 15%, calcium cations are present in a weight percent ranging from about 0.1% to about 5%, gelatin is present in a weight percent ranging from about 20% to about 50%, and trehalose is present in a weight percent from about 20% to about 50%, out of the total weight of the dry system. According to a certain embodiment, LMP having DE of about 7% and DA being null is present in the system in a weight percent ranging from about 5% to about 10%, calcium cations are present in a weight percent ranging from about 0.1% to about 5%, gelatin is present in a weight percent ranging from about 40% to about 50%, and trehalose is present in a weight percent ranging from about 40% to about 50%, out of the total weight of the dry system.

According to some embodiments, LMP, gelatin, and trehalose for the suspended release of the BALOs are present at a ratio of weight percent ranging from 1:1:1 to 1:10:10 in the dry system. According to certain embodiments, LMP, gelatin, and trehalose are present at a ratio of about 1:5:5 in the dry system. Preferably, the LMP has DE of about 7% and DA being null.

According to some embodiments, the dried system preserves viability of the immobilized BALOs at a density of at least about 1.0×10⁸ PFU/(g carriers) for at least 3 months, for at least 6 months, or for at least 12 months, at a temperature of up to about 25° C. According to further embodiments, the dried system preserves viability of the immobilized BALOs at a density of at least about 1.0×10⁸ PFU/(g carriers) for at least 3 months, for at least 6 months, or for at least 12 months, at a temperature ranging from about −20° C. to about 25° C. Each possibility represents a separate embodiment of the invention. According to an exemplary embodiment, the dried system comprising κ-carrageenan and trehalose preserves viability of the immobilized BALOs at a density of at least about 1.0×10¹⁰ PFU/(g carriers) for at least 12 months at a temperature of about 25° C. According to another exemplary embodiment, the dried system comprising LMP, gelatin, and trehalose preserves viability of the immobilized BALOs at a density of at least about 1.0×10⁸ PFU/(g carriers) for at least 12 months at a temperature of about 4° C.

According to another aspect, the present invention provides a method of preventing, reducing or eliminating a phytopathogenic bacterial disease in a plant, plant part, or a crop pre- or post-harvest comprising contacting the plant, the plant part, or the crop pre- or post-harvest with an effective amount of the delivery system according to the principles of the present invention, thereby preventing, reducing or eliminating the phytopathogenic bacterial disease in said plant, plant part, or crop.

According to some embodiments, the phytopathogenic bacterial disease is caused by phytopathogenic bacteria selected from the group consisting of Pectobacterium carotovorum, Pectobacterium chrysanthemi, Pectobacterium aroidearum, Pectobacterium atrosepticum Pectobacterium betavasculorum, Pectobacterium cacticida, Pectobacterium colocasium, Pectobacterium cypripedii, Pectobacterium melonis, Pectobacterium parmentieri, Pectobacterium Polaris, Pectobacterium punjabense, Pectobacterium rhapontici, Pectobacterium versatile, Pectobacterium wasabiae, Pectobacterium zantedeschiae, Xanthomonas campestris, Pseudomonas syringae, Pseudomonas fluorescens, Pseudomonas tolaasii, Pseudomonas glycinea, Erwinia amylovora, Ralstonia solanacearum, Acidovorax citrulli, Acidovorax oryzae, Acidovorax avenae, Dickeya solani, Dickeya chrysanthemi, Dickeya dadantii, Xylella fastidiosa, and Pantoea sp. Each possibility represents a separate embodiment of the invention.

According to some embodiments, the phytopathogenic bacterial disease is a macerating disease. According to further embodiments, the macerating disease is selected from the group consisting of soft rot, black leg, and aerial stem rot. According to an exemplary embodiment, the phytopathogenic bacterial disease is soft rot caused by Pectobacterium carotovorum.

According to yet further embodiments, the plant or the plant part is selected from the group consisting of a seed, seedling, plantlet, bulb, rhizome, tree, herb, shrub, creeper, vegetable, fruit, legume, mushroom, flower, and combinations thereof. Each possibility represents a separate embodiment of the invention.

According to certain embodiments, the plant, plant part or crop is selected from the group consisting of potato (Solanum tuberosum), onion, carrot, soybean, mushroom, and flower. According to an exemplary embodiment, the crop is potato and the phytopathogenic bacterial disease is soft rot.

According to some embodiments, contacting the plant, plant part, or crop with the system is performed by a process selected from the group consisting of a foliar application, soil application, application through irrigation systems, starter solutions, injection to soil, patch application, aerial application, and any combination thereof. Each possibility represents a separate embodiment of the invention.

According to another aspect, the present invention provides a method of reducing bacterial load of an item in at least one of pharmaceutical industry, microbiological industry, medicinal industry, food industry, marine industry, paper industry, textile, plant nurseries, aquaculture, soil treatments, ship maintenance, water treatment facilities, sewage treatment facilities, and water distribution systems, the method comprising contacting the item with an effective amount of the delivery system according to the principles of the present invention, thereby reducing the bacterial load of said item.

According to some embodiments, the method of reducing bacterial load of an item comprises preventing or arresting biofilm formation and/or reducing or eradicating mature biofilm. According to additional embodiments, the bacterial load is caused by bacteria selected from the group consisting of gram-negative bacteria, gram-positive bacteria, antibiotic resistant bacteria, and any combination thereof. According to further embodiments, the gram-negative bacteria are pathogenic gram-negative bacteria including, but not limited to, Escherichia coli, Pseudomonas, Klebsiella, Plesiomonas shigelloides, Salmonella, Shigella, Vibrio spp., such as Vibrio cholerae, Vibrio parahaemolyticus, Vibrio vulnificus, Yersinia enterocolitica, and Yersinia pseudotuberculosis. Each possibility represents a separate embodiment of the invention.

According to another aspect, there is provided a method for the preparation of a delivery system for the differential delivery of viable Bdellovibrio-and-like organisms (BALOs), wherein the delivery system comprises a carrier comprising a polysaccharide hydrocolloid and BALOs immobilized within the carrier, wherein the BALOs are in a physiological state of bdelloplasts, bdellocysts, and APs, which are present at a cell density of at least about 1.0×10⁸ PFU/(g carriers), the method comprising:
(i) admixing a suspension comprising BALOs with a hydrocolloid solution comprising the polysaccharide hydrocolloid to form a hydrocolloid composition comprising the BALOs; and
(ii) admixing the hydrocolloid composition comprising the BALOs of step (i) with a crosslinking solution to form the polysaccharide hydrocolloid in a wet gel form, thereby forming the delivery system in a wet form.

According to some embodiments, the method for the preparation of the delivery system further comprises a step of drying the delivery system of step (ii), thereby forming a dry delivery system.

According to further embodiments, drying the delivery system in the gel form is performed by at least one of an air drying, desiccant drying, vacuum drying, fluidized bed drying, spray-drying, sun drying and freeze-drying. According to a certain embodiment, drying the delivery system is performed by vacuum drying.

According to yet further embodiments, the method further comprises a step of grinding, crushing, pulverizing and/or any process known in the art useful for reducing the Feret diameter of a granule, bead, capsule, or particle.

According to still further embodiments, admixing the hydrocolloid composition comprising the BALOs with the crosslinking solution is performed by at least one of dripping, spraying, emulsification, and dispersion in oil followed by chilling. According to an exemplary embodiment, admixing the hydrocolloid composition comprising the BALOs with the crosslinking solution is performed by dripping.

According to further embodiments, the polysaccharide hydrocolloid for the preparation of the delivery system is selected from the group consisting of κ-carrageenan, pectin, alginate, agar, gellan, carboxymethyl cellulose (CMC), furecellaran, xanthan gum, locust bean gum (LBG), konjac-mannan, chitosan, pullulan, curdlan and derivatives and combinations thereof. According to one exemplary embodiment, the polysaccharide hydrocolloid is κ-carrageenan. According to another exemplary embodiment, pectin is low methoxyl pectin (LMP).

According to yet further embodiments, the polysaccharide hydrocolloid for the preparation of the delivery system is present in the hydrocolloid solution in a weight percent ranging from about 0.25% to about 10% out of the total weight of the hydrocolloid solution. According to still further embodiments, the polysaccharide hydrocolloid is present in the hydrocolloid solution in a weight percent ranging from about 1% to about 5% out of the total weight of the hydrocolloid solution. According to a certain embodiment, the polysaccharide hydrocolloid is present in the hydrocolloid solution in a weight percent of about 1% to about 3% out of the total weight of the hydrocolloid solution.

According to still further embodiments, a volumetric ratio between the BALOs suspension and the hydrocolloid solution ranges from about 1:3 to about 1:20, preferably from about 1:4 to about 1:9.

According to some embodiments, the hydrocolloid composition for the preparation of the delivery system further comprises a protein hydrocolloid and/or a protein. According to further embodiments, the protein hydrocolloid or protein are selected from the group consisting of gelatin, collagen, collagen hydrolysate, albumin, and any combination thereof. According to yet further embodiments, gelatin is present in the hydrocolloid solution in a weight percent ranging from about 0.25% to about 15% out of the total weight of the hydrocolloid solution. According to yet further embodiments, gelatin is present in a weight percent ranging from about 1% to about 10% out of the total weight of the hydrocolloid solution. According to a certain embodiment, gelatin is present in a weight percent of about 5% out of the total weight of the hydrocolloid solution.

According to some embodiments, the hydrocolloid composition for the preparation of the delivery system further comprises a water-absorbing agent. According to further embodiments, the water-absorbing agent is selected from the group consisting of starch, polyvinylpyrrolidone (PVP), and derivatives and combinations thereof. According to yet further embodiments, the water-absorbing agent is present in the hydrocolloid solution in a weight percent ranging from about 0.25% to about 15% out of the total weight of the hydrocolloid solution.

According to some embodiments, the hydrocolloid composition for the preparation of the delivery system further comprises at least one osmoprotectant.

According to further embodiments, the at least one osmoprotectant is selected from the group consisting of trehalose, inositol, glycerol, arabitol, betaine, an amino acid, and combinations thereof. According to yet further embodiments, the osmoprotectant is present in the hydrocolloid solution in a weight percent ranging from about 0.25% to about 15% out of the total weight of the hydrocolloid solution, alternatively in a weight percent ranging from about 1% to about 10% out of the total weight of the hydrocolloid solution. According to a certain embodiment, the osmoprotectant is present in the hydrocolloid composition in a weight percent of about 5% out of the total weight of the hydrocolloid solution.

According to some embodiments, the crosslinking solution comprises a metal cation and/or a gelation-inducing agent.

According to further embodiments, the crosslinking solution and the hydrocolloid composition are admixed at a volumetric ratio of from about 10:1 to about 500:1. According to a certain embodiment, the crosslinking solution and the hydrocolloid composition are admixed at a volumetric ratio of about 100:1.

According to yet further embodiments, the metal cation present in the crosslinking solution useful for the preparation of the system is selected from the group consisting of a potassium (K⁺), calcium (Ca²⁺), barium (Ba²⁺), magnesium (Mg²⁺), aluminum (Al³⁺), zinc (Zn²⁺), lead (Pb²⁺), ferrous (Fe²⁺), strontium (Sr²⁺), copper (Cu²⁺), cadmium (Cd²⁺), nickel (Ni²⁺), cobalt (Co²⁺), and combinations thereof. According to an exemplary embodiment, when the polysaccharide hydrocolloid is κ-carrageenan, the cross-linking solution comprises potassium ions as metal cations. According to another exemplary embodiment, when the polysaccharide hydrocolloid is LMP, the cross-linking solution comprises calcium ions as metal cations.

According to still further embodiments, the crosslinking solution comprises the metal cation in a weight percent ranging from about 0.1% to about 5% out of the total weight of the crosslinking solution.

According to some embodiments, the drying is performed at a temperature ranging from about 10° C. to 60° C. and/or at a pressure ranging from 10 mbar to 1000 mbar. According to an exemplary embodiment, the drying is performed at a temperature ranging from about 40° C. at a pressure of about 400 mbar.

According to some embodiments, following the step of drying, the system comprises at least about 1.0×10⁸ (PFU/g carriers) BALOs.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F present digital images acquired under a binocular (model 5ZX16, Olympus America Inc., Center Valley, PA, USA) of dried K-carrageenan carrier (350 μm in diameter) before (FIG. 1A) and after immersion in excess DW for 2.5 min, 5 min, 7.5 min, 10 min, and 12.5 (FIGS. 1B-1F, respectively). Scale bar=200 μm.

FIG. 2 presents histograms showing the concentration of potassium released from dried K-carrageenan carriers immersed in DW over time. n=6. (Student's 1-test, P<0.05).

FIGS. 3A-3B show the effect of predator cell type and added osmoprotectant on the entrapment efficiency (FIG. 3A) and the survival of the predator after vacuum-drying (FIG. 3B). Results represent the average of two independent experiments with three technical replicates for each treatment. n=5. (Student's t-test, P<0.05).

FIGS. 4A1-4D4 show SEM micrographs. FIGS. 4A1-4A4: K-carrageenan carrier, FIGS. 4B1-4B4: K-carrageenan carrier+bdelloplasts, FIGS. 4C1-4C4: K-carrageenan carrier with trehalose+bdelloplasts, FIGS. 4D1-4D4: K-carrageenan carrier with glycerol+bdelloplasts. In each row: FIGS. 4A1-4D1: an intact carrier, FIGS. 4A2-4D2: a close-up of the outer side of the carrier, FIGS. 4A3-4D3: a cut carrier, showing the internal part, FIGS. 4A4-4D4: a close-up on the inner side of the carrier.

FIGS. 5A-5B depict the growth and predation dynamics of released encapsulated B. bacteriovorus HD100-Td-tomato in dry κ-carrageenan carriers on E. coli ML35. The prey population was tracked by optical density (OD) (FIG. 5A) and the predatory population was tracked by fluorescence (relative fluorescence units, RFU) (FIG. 5B). Two independent experiments with three technical replicates for each treatment were conducted.

FIGS. 6A-6B depict the growth and predation dynamics of released encapsulated B. bacteriovorus HD100-Td-tomato in dry κ-carrageenan carriers on P. carotovorum subsp. carotovorum WPP14. The prey population was tracked by optical density (OD) (FIG. 6A) and the predatory population was tracked by fluorescence (relative fluorescence units, RFU) (FIG. 6B). Two independent experiments with three technical replicates for each treatment were conducted.

FIGS. 7A-7B depict the growth and predation dynamics of released encapsulated bdelloplasts of B. bacteriovorus HD100-Td-tomato in dry κ-carrageenan carriers on Pectobacterium carotovorum subsp. brasiliense. The prey population was tracked by optical density (OD) (FIG. 7A) and the predatory population was tracked by fluorescence (relative fluorescence units, RFU) (FIG. 7B). Two independent experiments with three technical replicates for each treatment were conducted.

FIGS. 8A-8B depict the growth and predation dynamics of released encapsulated bdelloplasts of B. bacteriovorus HD100-Td-tomato in dry κ-carrageenan carriers on Dickeya solani. The prey population was tracked by optical density (OD) (FIG. 8A) and the predatory population was tracked by fluorescence (relative fluorescence units, RFU) (FIG. 8B). Two independent experiments with three technical replicates for each treatment were conducted.

FIG. 9 shows the effect of carrier preparation conditions on the survival of encapsulated bdelloplasts with or without trehalose. Results represent the average of two independent experiments with three technical replicates for each treatment. n=5. (Student's t-test, P<0.05).

FIGS. 10A-10F are SEM (A-D) and high-resolution SEM (E-F) micrographs of κ-carrageenan-trehalose carrier encapsulating predators prepared with 0.5% KCl. An intact carrier (FIG. 10A), a close-up of the outer side of the carrier (FIG. 10B), the internal part of the carrier (FIG. 10C), a close-up of the inner side of the carrier (FIG. 10D), Bdelloplasts and AP cells (marked with black and white arrows, respectively) encapsulated within the carrier (FIG. 10E), and AP cells encapsulated within the carrier (FIG. 10F).

FIGS. 11A-11C depict the effect of encapsulated bdelloplasts of B. bacteriovorus HD100 in-carrageenan-trehalose carriers applied 60 min before application of P. carotovorum subsp. brasiliense (Pcb) on the development of soft-rot disease in potato slices: maceration area (cm²) (FIG. 11A), number of diseased slices (FIG. 11B), photographs of potato slices with a macerated tissue appears as dark patches (FIG. 11C). Error bars represent the standard error of 18 replicates. (Student's t-test, P<0.05).

FIGS. 12A-12C depict the effect of κ-carrageenan-trehalose carriers (without predators) applied 60 min before application of P. carotovorum subsp. brasiliense (Pcb) on the development of soft-rot disease in potato slices: maceration area (cm²) (FIG. 12A), number of diseased slices (FIG. 12B), photographs of potato slices with a macerated tissue appears as dark patches (FIG. 12C). Error bars represent the standard error of 18 replicates. (Student's 1-test, P<0.05).

FIGS. 13A-13B show the biofilm disruption of Pseudomonas fluorescens (FIG. 13A) and E-coli ZK2686 (FIG. 13B) by κ-carrageenan carriers encapsulating bdelloplasts of B. bacteriovorus HD100. The results are averages of 6 repetitions+SD from two different batches. (Student's t-test, P<0.05).

FIGS. 14A-14C present growth curves of P. carotovorum subsp. brasiliense (Pc1692, Pc3; FIGS. 14A and 14B, respectively) and carotovorum (WPP14; FIG. 14C) on 3 different LMP solutions (106 S-YA, 104 AS and 5 CS) as single carbon sources. Results are averages of 10 replicates±SD from two different batches.

FIGS. 15A-15C present growth curves of P. carotovorum subsp. brasiliense (Pc1692, Pc3; FIGS. 15A and 15B, respectively) and carotovorum (WPP14; FIG. 15C) on 3 different LMP carriers (106 S-YA, 104 AS and 5 CS) as a single carbon source. Results are averages of 10 replicates±SD from two different batches.

FIGS. 16A-16C depict the activity of extracellular PCWDEs (Pel, Peh and Prt) secreted by P. carotovorum subsp. Brasiliense (Pc1692, Pc3; FIGS. 16A and 16B, respectively), and carotovorum (WPP14; FIG. 16C) after exposure to LMP solutions (106 S-YA, 104 AS and 5 CS) as a single carbon source. Results are averages of 10 replicates±SD from two different batches. (Student's t-test, P<0.05).

FIGS. 17A-17D depict the attraction of GFP-tagged Pcb to different LMP 5 CS, 104 AS, and 106 S-YA carriers. Experimental system (FIG. 17A). Fluorescent binocular images after 48 h of GFP-tagged Pcb to LMP 5 CS (FIG. 17B), 104 AS (FIG. 17C), or 106 S-YA (FIG. 17D). Scale bar=1 cm.

FIGS. 18A-18F depict the degradation of the different LMP carriers by P. carotovorum subsp. brasiliense (Pcb). Representative digital images of carriers' size and appearance (×2 magnification): 5 CS (FIGS. 18A, 18D), 104 AS (FIGS. 18B, 18E), 106 AS-YA (FIGS. 18C, 18F); incubated for 72 h in DDNB, without (FIGS. 18A-18C) or with Pcb (FIGS. 18D-18F) were acquired under a binocular (model 5ZX16, Olympus America Inc., Center Valley, PA, USA). Percent weight change of the carriers in DDNB without or with Pcb (FIG. 18G). Results are averages of 6 replicates+SD from two different batches. (Student's 1-test, P<0.05).

FIGS. 19A-19E depict the degradation of 5 CS carriers by P. carotovorum subsp. brasiliense (Pcb). Representative digital images of differently sized carriers (×2 magnification) in DDNB for 72 h: 3 mm (FIG. 19A-19B), 2 mm (FIG. 19C-19D), without (FIG. 19A, FIG. 19C) or with Pcb (FIG. 19B, FIG. 19D), were acquired under a binocular (model 5ZX16, Olympus America Inc., Center Valley, PA, USA). Percent weight change of the carriers in DDNB without or with Pcb (FIG. 19E). Results are averages of 6 replicates±SD from two different batches. (Student's t-test, P<0.05).

FIGS. 20A-20G depict the degradation of 5 CS-gelatin carriers by P. carotovorum subsp. Brasiliense (Pcb). Representative digital images of carriers (×2 magnification) with gelatin added at 0% (FIGS. 20A, 20B), 2% (FIGS. 20C, 20D), and 5% (FIGS. 20E, 20F) in DDNB for 72 h, without (FIGS. 20A, 20C, 20E) or with Pcb (FIGS. 20B, 20D, 20F) were acquired under a binocular (model 5ZX16, Olympus America Inc., Center Valley, PA, USA). Percent weight change of the carriers in DDNB without or with Pcb (FIG. 20G). Results are averages of 6 replicates±SD from two different batches. (Student's t-test, P<0.05). ND, not determined.

FIGS. 21A-21D depict the degradation of dried 5 CS-gelatin carriers by P. carotovorum subsp. brasiliense (Pcb). Representative digital images (×2 magnification) of dry carrier (FIG. 21A), dry carrier in DDNB for 72 h without (FIG. 21B) or with Pcb (FIG. 21C) were acquired under a binocular (model 5ZX16, Olympus America Inc., Center Valley, PA, USA). Percent weight change of the carriers in DDNB without or with Pcb (FIG. 21D). Results are averages of 6 replicates±SD from two different batches. (Student's t-test, P<0.05). ND, not determined.

FIGS. 22A-22F are SEM micrographs (FIGS. 22A-D) and high-resolution SEM (FIGS. 22E, 22F) of optimized dried carriers comprising LMP and gelatin encapsulating B. bacteriovorus predators: an intact carrier (FIG. 22A). The outer surface of the carrier (FIG. 22B). A carrier cut in half, showing a thin shell wall and a hollow core (FIG. 22C). The inner surface of the carrier (FIG. 22D). AP cells and bdelloplasts embedded on the surface of the carrier (FIGS. 22E, 22F). Black and white arrows point to bdelloplasts and AP cells, respectively.

FIGS. 23A-23B depict the growth and predation efficiency on P. carotovorum subsp. brasiliense (Pcb) prey of encapsulated B. bacteriovorus HD100-Td-tomato in optimized dried carriers comprising LMP and gelatin with 1, 2 or 3 carriers per well. Optical density (OD, 600 nm) of the prey populations (FIG. 23A). Td-tomato fluorescence (relative fluorescence units, RFU) of the predator population (FIG. 23B). Two independent experiments each with three technical replicates for each treatment were conducted.

FIG. 24 depicts the long-term survival of B. bacteriovorus HD100 in carrageenan-trehalose and pectin-gelatin-based carriers, and in liquid suspensions at different temperatures. Results represent the average of two independent experiments with three technical replicates for each treatment.

FIGS. 25A-25D depict the protection of potato tubers against Pectobacterium brasiliense by hydrated Bdelovibrio formulation based on κ-carrageenan-trehalose under controlled conditions. The tubers were dipped in P. brasiliense suspension and treated with the predators as follows: control (water only); Pb1692 inoculation followed by sterile tap water (Pecto only); pathogen inoculation and application of predators as freshly grown AP cells or as encapsulated predators released from carriers. FIG. 25A depicts the percentage of tuber decay (by weight). FIG. 25B depicts the disease index. FIG. 25C is a photograph of the potato tubers in planting trays, after Pb1692 inoculation, predator treatments, and prior to covering with planting soil. FIG. 25D depicts the number of infected potatoes. Results represent the average±SD of eight independent experiments with 15 replicates for each treatment. [[IS—Treatments not connected by the same letter are significantly different-DELETE]] (Student's t-test, P<0.05).

FIGS. 26A-26E show the protection of potato tubers in pots under natural net-house conditions against Pectobacterium brasiliense by hydrated Bdelovibrio formulation based on κ-carrageenan-trehalose. FIG. 26A is a photograph of the pot plants post Pb1692 inoculation in the net-house. FIG. 26B depicts the number of infected potatoes at the end of the 4-week period with the following treatments: control (water only); Pb1692 inoculation followed by sterile tap water (Pecto only); pathogen inoculation and application of predators as freshly grown AP cells or as predators released from carriers. FIG. 26C is a photograph of cut potato tubers at the end of the experiment. FIG. 26D depicts the percentage of tuber decay (by weight). FIG. 26E depicts the disease index. Results represent the average±SD of two independent experiments with 32 replicates for each treatment. [[IS-Treatments not connected by the same letter are significantly different-DELETE]] (Student's t-test, P<0.05).

### DETAILED DESCRIPTION OF THE INVENTION

The present invention provides a system for differential delivery of viable Bdellovibrio-and-like organisms (BALOs) comprising a carrier comprising a polysaccharide hydrocolloid, and BALOs immobilized within the carrier, wherein the BALOs are in a physiological state of AP cells, bdellocysts and bdelloplasts, which are present at a cell density of at least 1.0×10⁸ PFU/(g carriers), wherein the polysaccharide hydrocolloid is in a wet or dried-gel form, and wherein the carrier preserves viability and bacteriolytic efficiency of the immobilized BALOs. The present invention further provides uses of the delivery systems and methods of preparation thereof.

### Delivery Systems

The terms “system for differential delivery of viable Bdellovibrio-and-like organisms (BALOs)”, “delivery system”, “system”, and “BALOs' delivery system” are used herein interchangeably and denote the system of the present invention.

The term “bdellovibrio-and-like organisms” (BALOs), as used herein, are gram-negative, small, obligate predators of other gram-negative bacteria. These bacteria interact with their prey as highly motile attack phase cells, attaching to the outer membrane and consuming the prey extracellularly (epibiotic predation) or penetrating their periplasm (periplasmic predation). The former divide in a binary fashion, while the latter grow as a polynucleotide filament to finally split as progeny attack cells. Most BALO isolates, including Bdellovibrio bacteriovorus, are periplasmic predators. According to certain embodiments, BALOs comprise or consist of Bdellovibrio bacteriovorus, Bdellovibrio stolpii and Bdellovibrio starrii.

The term “hydrocolloid”, as used herein, refers to water-soluble polymers of vegetable, animal, microbial or synthetic origin, that generally contain multiple hydroxyl, carboxyl, amine and/or amide groups.

The terms “polysaccharide hydrocolloid” and “protein hydrocolloid”, as used herein, refer to a polysaccharide or protein, respectively, capable of thickening aqueous solutions by increasing viscosity values or forming a gel in an aqueous medium. Gel formation is the phenomenon involving the association or cross-linking of the polymer chains to form a three-dimensional network that traps or immobilizes the water within it to form a rigid structure that is resistant to flow. Gel formation may be spontaneous or induced by using a suitable cross-linking agent or when combined with additional hydrocolloids at defined conditions and concentrations. For example, the combination of xanthan gum and locust bean gum (LBG), both non-gelling hydrocolloids, leads to the formation of a firm gel.

The term “bdelloplasts”, as used herein, refers to a modified gram-negative cell formed in the host of a parasitic Bdellovibrio bacteriovorus bacterium. Bdelloplasts are generated by penetrating the periplasm of gram-negative bacteria, which constitute the main prey for BALOs. After prey invasion, the prey peptidoglycan is modified, producing a bdelloplast. Acylation of the prey peptidoglycan by long-chain fatty acids and solubilization of 25% of the lipopolysaccharide (LPS) glucosamine by a lipopolysaccharidase activity increase bdelloplast hydrophobicity and stabilize the outer membrane, which now acts as an osmotic barrier. Thus, bdelloplasts can offer enhanced protection compared to naked AP cells during dehydration of the carriers, in which the osmolarity is expected to increase.

The term “bdellocysts”, as used herein, refers to bacterial cells of the Bdellovibrio sp. which infect a prey and produce therein resting cells, known as bdellocysts. Bdellocysts typically contain more deoxyribonucleic acid (DNA), ribonucleic acid (RNA), protein, and carbohydrate than attack phase cells. Bdellocysts are more resistant than attack phase cells to effects of elevated temperatures, sonic treatment, and desiccation.

The term “immobilized”, as used herein, refers to bacterial cells entrapped, embedded, and/or encapsulated within the polysaccharide hydrocolloid, and released therefrom by disintegration, dissolution and/or degradation of the hydrocolloid.

According to some embodiments, the immobilized BALOs, e.g., bdelloplasts, are present at a cell density of at least 1.0×10⁸ PFU/(g carriers). According to further embodiments, the immobilized BALOs, e.g., bdelloplasts, are present at a cell density of at least 1.0×10⁹ PFU/(g carriers). According to yet further embodiments, the immobilized BALOs, e.g., bdelloplasts, are present at a cell density of at least 1.0×10₁₀ PFU/(g carriers).

The term “wet form”, as used herein, refers to a gel formed by the polysaccharide hydrocolloid, which contains at least 75 wt % water out of the total weight of the hydrocolloid or gel. According to further embodiments, the term “wet form”, refers to a carrier prepared from an aqueous solution that has not undergone drying

The term “dried-gel form”, as used herein, refers to a gel formed by the polysaccharide hydrocolloid and then dried such that it contains less than about 10 wt % water out of the total weight of the dried gel.

The term “preserves viability”, as used herein, refers to at least about 1.0×10⁸ PFU/(g carriers) of viable BALOs which are immobilized within the carrier after drying. In some embodiments, the term “preserves viability” refers to bdelloplasts and/or AP cells, which remain viable following release from the carrier.

Quantitative measurements of cell viability can be performed by solubilizing gel carriers or dried carriers in sterile sodium hexametaphosphate (SHMP) or sterile DW, in order to release the encapsulated Bdellovibrio cells. PFUs, representing viable cells can be counted by dilution plating in double-layered agar.

The percentage of BALOs viable cells immobilized within the carrier is embodied in the encapsulation efficiency (EE) index and is calculated according to the following equation:

$$EE ( \% ) = ( N / {N}_{0} ) \times 100 , \qquad Equation I$$

where EE is the encapsulation efficiency, expressed in percentage, N is the number of cells released from the carriers (CFU/g of carriers), and N₀ is the number of cells in the hydrocolloid solution (CFU/g of suspension).

The term “preserves bacteriolytic efficiency”, as used herein, refers to BALOs which are immobilized within the carrier, released therefrom, and retain bacteriolytic efficiency of at least 10%, or at least 20%, 30%, 40%, 50%, 60%, 70%, 80%, or at least 90%, or 100% of the bacteriolytic activity of BALOs prior to immobilization. In further embodiments, the term “preserves bacteriolytic efficiency” refers to BALOs which retain bacteriolytic efficiency following release from the dried carrier. Bacteriolytic efficiency of the BALOs is preserved as long as the viability of the BALOs is preserved.

BALOs bacteriolytic efficiency following encapsulation within the carrier was examined using predation dynamics assays. Predation of soft rot bacteria by encapsulated BALOs in the dried carriers was tracked by fluorescence (predator growth) and OD (prey decay) in a plate reader incubated at 28° C.

According to some embodiments, the carrier is present as a granule, a bead, a capsule, a particle, a powder, and any combination thereof. Each possibility represents a separate embodiment of the present invention.

The Feret diameter of the carrier is measured along the longest chord thereof. According to some embodiments, the carrier has an average Feret diameter ranging from about 0.01 mm to about 10 mm. In further embodiments, the carrier has an average Feret diameter ranging from about 0.1 mm to about 5 mm. In yet further embodiments, the carrier has an average Feret diameter ranging from about 2 mm to about 4 mm.

According to some currently preferred embodiments, the polysaccharide hydrocolloid is in the dried-gel form. In such embodiments, the system is referred to as a “dry system” or “dried system”. Without wishing to be bound by any theory or mechanism of action, it is contemplated that the dried gel form of the hydrocolloid-based carrier provides extended shelf-life and stability of the encapsulated BALOs, as compared to the wet gel form. Furthermore, it has been surprisingly found that the dried gel carrier provided faster BALOs release upon contact with water as compared to the wet gel carriers. The inventors of the present invention have further shown that the dried hydrocolloid-based carriers are highly efficient for entrapment and fast release of the bacterial predator B. bacteriovorus, keeping cells viable, capable of preventing bacterial infections in plant tissues. It has been further unexpectedly discovered that the bacteriolytic efficiency of the bdelloplasts released from the dried gel carriers was higher as compared to that of the released AP cells. Therefore, the combination of the dried-gel carriers and the bdelloplast form of B. bacteriovorus provides the most efficient BALOs' delivery system.

The polysaccharide hydrocolloid can be a naturally occurring or a semi-synthetic polysaccharide hydrocolloid (e.g., chemically modified). The naturally occurring polysaccharide hydrocolloid can be obtained or extracted from various natural sources, such as, e.g., botanical, microbial, or animal sources. Polysaccharide hydrocolloids obtained/extracted from natural sources include, but are not limited to, κ-carrageenan, pectin, alginate, agar, gellan, furecellaran, xanthan gum, locust bean gum (LBG), konjac-mannan, chitosan, pullulan, curdlan, and derivatives and combinations thereof. Semi-synthetic polysaccharide hydrocolloids include, but are not limited to, carboxymethyl cellulose and other cellulose ethers, hydroxyethyl and propyl cellulose.

The type of hydrocolloid can be chosen based on the intended use of the BALOs' delivery system. For example, if the delivery system is to be used in the food industry, naturally occurring hydrocolloids, which are food-grade materials, should be used. Furthermore, the type of the hydrocolloid may be selected based on the desired release profile of the immobilized BALOs, as detailed hereinbelow.

Non-limiting examples of suitable polysaccharide hydrocolloids, which can be employed in the delivery system include k-carrageenan, pectin, alginate, agar, gellan, carboxymethyl cellulose (CMC), furecellaran, xanthan gum, locust bean gum (LBG), konjac-mannan, chitosan, pullulan, curdlan, and derivatives and combinations thereof.

The term “derivative” as used herein, includes salts, amides, esters, enol ethers, enol esters, acetals, ketals, orthoesters, hemiacetals, hemiketals, acids, bases, solvates, hydrates or prodrugs of a hydrocolloid.

In some exemplary embodiments, the carrier comprises κ-carrageenan.

Carrageenan is an intercellular matrix constituent in many species of red seaweed. Chemically, this polymer is a linear, anionic, sulfated galactan which is composed of repeating galactose units and 3,6-anhydrogalactose, both sulfated and nonsulfated, linked by alternating α-(1-3)- and β-(1-4)-glycosidic linkages (Nussinovitch & Hirashima, 2013). Depending on the number and position of the sulfate groups, three main carrageenans have been identified: iota (1), kappa (κ) and lambda (λ). Both κ- and ι-carrageenan dispersions undergo sol-gel transition upon cooling and adding counter ions such as K⁺ and Ca²⁺ (Thrimawithana, Young, Dunstan, & Alany, 2010). Carrageenans are used for their gelling, thickening and stabilizing abilities in a variety of commercial applications, particularly in food products and sauces (Necas & Bartosikova, 2013).

In some exemplary embodiments, the carrier comprises pectin.

Pectin is an anionically charged structural plant polysaccharide consisting of a linear backbone of a (1-4)-D-galacturonic acid residue, which can be partly methyl-esterified or amidated. Therefore, pectin is characterized by its degree of esterification (DE) as well as its degree of amidation (DA). Low methoxylated pectin (also termed low-methoxyl-pectin, LMP) (DE<50%), amidated or not, can form a gel in the presence of divalent cations. Pectin has been widely used as a delivery vehicle for colon-specific oral drugs and, to a lesser extent, probiotics (low-methoxyl-pectin and rice-bran).

According to some embodiments, pectin is a low methoxyl pectin (LMP).

According to some embodiments, the carrier comprises a LMP having a degree of esterification that ranges between about 5% and 30% and a degree of amidation that ranges between about 0% and 25%. According to further embodiments, the carrier comprises LMP having a degree of esterification that ranges between about 5% and 10% and a degree of amidation that ranges between about 0% and 5%. In certain embodiments, the carrier comprises a non-amidated LMP.

According to some embodiments, the polysaccharide hydrocolloid is present in the system in a weight percent ranging from about 2% to about 25% out of the total weight of the dry system.

According to some embodiments, the polysaccharide hydrocolloid is present in the system in a weight percent ranging from about 0.5% to about 25% out of the total weight of the wet system. In further embodiments, the hydrocolloid is present in the system in a weight percent ranging from about 0.5% to about 5% out of the total weight of the wet system. In yet further embodiments, the hydrocolloid is present in the system in a weight percent ranging from about 1% to about 3% out of the total weight of the wet system.

According to some embodiments, the polysaccharide hydrocolloid is crosslinked by a crosslinking agent and/or combined with a gelation-inducing agent. The most common type of hydrocolloid crosslinking agents are metal ions, and, in particular, monovalent or divalent metal ions. In some embodiments, the crosslinking agent comprises a metal cation selected from the group consisting of a calcium (Ca²⁺), barium (Ba²⁺), magnesium (Mg²⁺), aluminum (Al³⁺), potassium (K⁺), zinc (Zn²⁺), lead (Pb²⁺), ferrous (Fc²⁺), strontium (Sr²⁺), copper (Cu²⁺), cadmium (Cd²⁺), nickel (Ni²⁺), cobalt (Co²⁺), and combinations thereof. Each possibility represents a separate embodiment of the present invention. In some exemplary embodiments, carriers comprising κ-carrageenan are crosslinked by potassium (K⁺) ions. In further exemplary embodiments, carriers comprising LMP are crosslinked by (Ca²⁺) ions.

In some embodiments, gelation-inducing agents include polycationic hydrocolloids and polycationic agents such as, for example, LBG, genipin, chitosan, polylysine, polyarginine, polyethylenimine (PEI), mannosylated PEI, and derivatives and combinations thereof. Each possibility represents a separate embodiment of the present invention.

According to some embodiments, the carrier further comprises a water-absorbing agent. The term “water-absorbing agent”, as used herein, refers to an agent that is configured to enhance the absorbance of water by the carrier.

In some embodiments, the carrier comprises pectin as the polysaccharide hydrocolloid. In some embodiments, the carrier further comprises gelatin as a protein hydrocolloid. Without wishing to be bound by any theory or mechanism of action, it is contemplated that gelatin forms ionic interactions with pectin dependent on pH, ionic strength, and type of pectin, thereby helping to overcome the reduced stability when using a low concentration of pectin in the carrier.

According to some embodiments, gelatin is present in the system in a weight percent ranging from about 20% to about 85% out of the total weight of the dry system.

In further embodiments, gelatin is present in the system in a weight percent ranging from about 20% to about 50% out of the total weight of the dry system. In yet further embodiments, gelatin is present in the system in a weight percent ranging from about 40% to about 50% out of the total weight of the dry system.

According to some embodiments, gelatin is present in the system in a weight percent ranging from about 0.25% to about 15% out of the total weight of the wet system.

According to some embodiments, the system and/or the carrier do not contain gelatin as a single hydrocolloid component. In further embodiments, the system and/or the carrier do not contain gelatin.

Dry pectin carriers have a dense structure that impedes pectinolytic activity, which can be overcome by the addition of gelatin or a water-absorbing agent, increasing swelling. In some embodiments, the water-absorbing agent is a hydrocolloid selected from the group consisting of starch, polyvinylpyrrolidone (PVP), polyacrylic and polymethacrylic compounds, vinyl polymers, polycarboxylic acids, polyethers, polyimines, polyamides, and derivatives and combinations thereof. Each possibility represents a separate embodiment of the present invention.

According to some embodiments, the water-absorbing agent is present in the system in a weight percent ranging from about 25% to about 85% out of the total weight of the dry system.

In further embodiments, the water-absorbing agent is present in the system in a weight percent ranging from about 35% to about 80% out of the total weight of the dry system. In yet further embodiments, the water-absorbing agent is present in the system in a weight percent ranging from about 45% to about 75% out of the total weight of the dry system.

According to some embodiments, the water-absorbing agent is present in the system in a weight percent ranging from about 0.25% to about 15% out of the total weight of the wet system.

According to some embodiments, the carrier further comprises at least one osmoprotectant. The term “osmoprotectant”, as used herein, refers to a compound that acts as an osmolyte and helps predator bacteria as described herein to survive osmotic stress. The osmoprotectant may be added to the carrier composition prior to desiccation to reduce/eliminate salt-induced stress and to improve survival of the encapsulated bacteria during dehydration. Osmoprotectants include a variety of compound classes, such as sugars, amino acids, polyols and heterosides.

The inventors of the present invention have surprisingly found that the higher the molecular weight of the added osmoprotectant, the higher the entrapment efficiency of BALOs within the carrier.

According to some embodiments, the molecular weight of the osmoprotectant is at least 50 g/mole. In further embodiments, the molecular weight of the osmoprotectant is at least 200 g/mole. In yet further embodiments, the molecular weight of the osmoprotectant is at least 300 g/mole.

According to some embodiments, the at least one osmoprotectant is selected from the group consisting of trehalose, inositol, glycerol, arabitol, betaine, mannitol, sucrose, an amino acid, and combinations thereof. Each possibility represents a separate embodiment of the present invention. In some embodiments, the at least one osmoprotectant is trehalose and/or glycerol. In certain embodiments, the at least one osmoprotectant is trehalose.

According to some embodiments, the at least one osmoprotectant is present in the system in a weight percent ranging from about 20% to about 85% out of the total weight of the dry system. According to alternative embodiments the at least one osmoprotectant is present in the system in a weight percent ranging from about 10% to about 90% of the total weight of the dry system.

According to some embodiments, the at least one osmoprotectant is present in the system in a weight percent ranging from about 0.25% to about 15% out of the total weight of the wet system.

According to some embodiments, the system comprises up to about 0.1% (w/w) water out of the total weight of the dry system. According to further embodiments, the system comprises up to about 0.5% (w/w) water out of the total weight of the dry system. According to yet further embodiments, the system comprises up to about 2.5% (w/w) water out of the total weight of the dry system.

According to some embodiments, the system comprises from about 0.1% (w/w) to about 10% (w/w) water out of the total weight of the dry system. In further embodiments, the system comprises from about 0.2% (w/w) to about 5% (w/w) water out of the total weight of the dry system. In yet further embodiments, the system comprises from about 0.5% (w/w) to about 2.5% (w/w) water out of the total weight of the dry system.

According to some embodiments, the system comprises from about 75% (w/w) to about 99% (w/w) water out of the total weight of the wet system.

As mentioned hereinabove, the dried hydrocolloid-based carriers are highly efficient in BALOs entrapment, preserving BALOs' viability and bacteriolytic efficiency. BALOs are bacterial predators that have an obligate requirement for gram-negative prey for their growth and replication. Release of BALOs, e.g., bdelloplasts, from said carriers activates this obligatory need to prey for their survival outside the carrier. Controlling BALO's predatory capability by the differential release of immobilized BALOs, e.g., bdelloplasts, from the carriers allows efficient predation to be achieved.

The system of the present invention is configured to release the immobilized viable BALOs, e.g., bdelloplasts, upon the carrier disintegration.

According to some embodiments, the differential delivery of BALOs, e.g., bdelloplasts, comprises a fast release or a suspended release of the viable BALOs, e.g., bdelloplasts, from the carrier. A differential delivery system with a fast predatory release profile is suitable for preventive antimicrobial treatments, while a system with a suspended predatory release can be beneficially used in targeted treatments. According to some embodiments, the carrier composition is tailored according to a desired release profile of the BALOs, e.g., bdelloplasts, from said carrier.

According to some embodiments, the carrier composition suitable for the fast release of the BALOs comprises κ-carrageenan and potassium cation.

According to some embodiments, the carrier comprising κ-carrageenan is configured to undergo swelling and dissolution/disintegration upon contact with an aqueous solution, thereby releasing the BALOs, e.g., bdelloplasts. According to certain embodiments, the aqueous solution is water/DW/DDW. Without wishing to be bound by any theory or mechanism of action, it is contemplated that swelling and disintegration behaviors of the κ-carrageenan-based dried carrier are due to a loss of potassium ions which are the main cross-linking agents and/or to an exchange of these cations with other monovalent cations present where the delivery system is applied. Without further wishing to be bound by any theory or mechanism of action, swelling and disintegration behaviors depend on the external volume of the aqueous solution that influences the potassium concentration gradient during diffusion, and on the carrier size which determines the surface area from which potassium ions can escape.

According to some embodiments, the disintegration of the carrier comprising κ-carrageenan occurs from about 1 min to about 48 h following contact with an aqueous solution. This fast disintegration can be particularly useful in aquaculture. In further embodiments, the disintegration of the carrier comprising κ-carrageenan occurs from about 1 min to about 12 h following contact with an aqueous solution. In yet further embodiments, the disintegration of the carrier comprising κ-carrageenan occurs from about 1 min to about 3 h following contact with an aqueous solution.

According to some embodiments, the carrier comprises κ-carrageenan and trehalose. According to some embodiments, k-carrageenan is present in the system in a weight percent ranging from about 5% to about 25% and trehalose is present in a weight percent ranging from about 25% to about 85%, out of the total weight of the dry system.

In further embodiments, κ-carrageenan is present in the system in a weight percent ranging from about 5% to about 25% and trehalose is present in a weight percent ranging from about 25% to about 80%, out of the total weight of the dry system. In yet further embodiments, κ-carrageenan is present in the system in a weight percent ranging from about 15% to about 25% and trehalose is present in a weight percent ranging from about 70% to about 80%, out of the total weight of the dry system.

According to some embodiments, k-carrageenan is present in the system in a weight percent ranging from about 0.25% to about 10% and trehalose is present in a weight percent ranging from about 0.25% to about 15%, out of the total weight of the wet system.

According to some embodiments, the carrier comprises κ-carrageenan and glycerol. According to some embodiments, k-carrageenan is present in the system in a weight percent ranging from about 5% to about 25% and glycerol is present in a weight percent ranging from about 25% to about 85%, out of the total weight of the dry system.

In further embodiments, κ-carrageenan is present in the system in a weight percent ranging from about 15% to about 25% and glycerol is present in a weight percent ranging from about 45% to about 80%, out of the total weight of the dry system. In yet further embodiments, κ-carrageenan is present in the system in a weight percent ranging from about 15% to about 25% and glycerol is present in a weight percent ranging from about 50% to about 80%, out of the total weight of the dry system.

According to some embodiments, κ-carrageenan is present in the system in a weight percent ranging from about 0.25% to about 10% and glycerol is present in a weight percent ranging from about 0.25% to about 15%, out of the total weight of the wet system.

According to some embodiments, the carrier composition suitable for the suspended release of BALOs, e.g., bdelloplasts, comprises LMP, gelatin or a water-absorbing agent, and calcium cation.

According to some embodiments, LMP is present in the system in a weight percent ranging from about 2% to about 25% and gelatin or the water-absorbing agent are present in a weight percent ranging from about 20% to about 85%, out of the total weight of the dry system. According to further embodiments, LMP is present in the system in a weight percent ranging from about 2% to about 15% and gelatin or the water-absorbing agent are present in a weight percent ranging from about 25% to about 80%, out of the total weight of the dry system.

According to some embodiments, LMP is present in the system in a weight percent ranging from about 0.25% to about 10% and gelatin or the water-absorbing agent is present in a weight percent ranging from about 0.25% to about 15%, out of the total weight of the wet system.

According to some embodiments, the carrier composition suitable for the suspended release of BALOs, e.g., bdelloplasts, comprises LMP, gelatin, trehalose, and calcium cation. According to further embodiments, LMP is present in the system in a weight percent ranging from about 2% to about 15%, gelatin is present in a weight percent ranging from about 20% to about 50%, and trehalose is present in a weight percent ranging from about 20% to about 50%, out of the total weight of the dry system. According to yet further embodiments, LMP is present in the system in a weight percent ranging from about 5% to about 10%, gelatin is present in a weight percent ranging from about 40% to about 50%, and trehalose is present in a weight percent ranging from about 40% to about 50%, out of the total weight of the dry system.

According to some embodiments, the carrier comprising LMP is configured to undergo disintegration by plant cell wall degrading enzymes (PCWDEs). PCWDEs and pectinases, in particular, are produced by soft rot Enterobacteria (SREs), which are gram-negative pathogenic bacteria, preyed upon by BALOs. Without wishing to be bound by any theory or mechanism of action, it is contemplated that the LMP-based immobilization system for BALOs according to the principles of the present invention employs PCWDE secretion by the pathogens and chemotaxis that is induced by pectin residues of the system, bringing upon the disintegration of the immobilization system and release of the encapsulated predators.

According to some embodiments, enzymatic degradation of the carrier comprising LMP occurs over a period of hours to days or weeks following contact with the PCWDEs in soil or in any medium. In further embodiments, disintegration of the carrier comprising LMP occurs from about 6 hours to about 84 hours following contact with the PCWDEs. In yet further embodiments, the disintegration of the carrier comprising LMP occurs from about 10 hours to about 72 hours following contact with the PCWDEs, alternatively from about 12 hours to about 36 hours, or for about 24 hours.

According to some embodiments, full decomposition of the polymer in actual environmental conditions may take a period of months. For example, full decomposition of the polymer in soil could take a period of 6 to 9 months.

The system can further include additives such as, but not limited to, anti-caking agents, anti-clamping agents, preservatives, antioxidants, adhesives, hydrophobic components, and resins.

According to some embodiments, the components of the delivery system, e.g., the polysaccharide hydrocolloid, the crosslinking agent, the water absorbing agent, and the osmoprotectant as well as the other optional additives, such as the anti-caking agent, the anti-clamping agent, the preservative, the antioxidant, the adhesive, the hydrophobic component, and the resin, are GRAS (generally recognized as safe), biodegradable, and non-toxic. According to further embodiments, the components of the delivery system are FDA approved.

### Uses of the Delivery Systems

The system of the present invention can be useful in agricultural applications to prevent bacterial soft rot infections in plant tissues or in crop. The inventors of the present invention have examined the efficacy of the system using a potato slices model as well as potato tubers and showed a decrease in soft rot disease incidence, smaller maceration area, and decrease in disease index after treatment of SRE-infected potato slices or potato tubers with the BALOs-encapsulating carrier.

Thus, the system of the present invention can be used in the treatment of a phytopathogenic bacterial disease in a plant, plant part, or crop, pre- or post-harvest.

In some embodiments, the phytopathogenic bacterial disease is caused by phytopathogenic bacteria that include, but are not limited to, Pectobacterium carotovorum, Pectobacterium chrysanthemi, Pectobacterium aroidearum, Pectobacterium atrosepticum Pectobacterium betavasculorum, Pectobacterium cacticida, Pectobacterium colocasium, Pectobacterium cypripedii, Pectobacterium melonis, Pectobacterium parmentieri, Pectobacterium polaris, Pectobacterium punjabense, Pectobacterium rhapontici, Pectobacterium versatile, Pectobacterium wasabiae, Pectobacterium zantedeschiae, Xanthomonas campestris, Pseudomonas syringae, Pseudomonas fluorescens, Erwinia amylovora, Ralstonia solanacearum, Acidovorax citrulli, Acidovorax oryzae, Acidovorax avenae, Dickeya solani, Dickeya chrysanthemi, Dickeya dadantii, Dickeya pectinolytic, and Xylella fastidiosa. Each possibility represents a separate embodiment of the present invention.

In some embodiments, the crop is selected from the group consisting of potato (Solanum tuberosum), onion (dry bulb and green), welsh onion (Allium fistulosum), shallot, carrot, soybean, barley, buckwheat, millet, oats, rice, rye, quinoa, sugar beet, teff, teosinte, triticale, wheat, wild rice, corn, soybean, cotton, borage, buffalo gourd (Cucurbita foetidissima), canola, crambe, flax, jojoba, lesquerella, oilseed rape, safflower, sesame, sunflower, grain sorghum (milo), sugar cane, aloe vera, asparagus, bamboo shoots, globe artichoke, okra, peanut, pineapple, strawberry, allspice, angelica, star anise, annatto (seed), balm, basil, burnet, chamomile, caper buds, caraway, black caraway, cardamom, cassia bark, cassia buds, catnip, celery seed, chervil (dried), chive, Chinese chive, cinnamon tree, clary, clove buds, coriander leaf (cilantro or Chinese parsley), coriander seed (cilantro), costmary, culantro (leaf), culantro (seed), cumin, curry (leaf), dill (dillweed), dill (seed), epazote, fennel seed, fenugreek, white ginger flower, grains of paradise (Aframomum melegueta), horehound (Marrubium vulgare), hyssop, juniper berry, lavender, lemongrass, lovage (leaf and seed), mace, marigold, marjoram (including oregano), mexican oregano, mioga flower, mustard (seed), nasturtium, nutmeg, parsley, pennyroyal, pepper (black and white), pepper leaves, peppermint, perilla, poppy (seed), rosemary, rue, saffron, sage, savory (summer and winter), spearmint, stevia leaves, sweet bay, tansy, tarragon, thyme, vanilla, wintergreen, woodruff, wormwood, broccoli, Chinese broccoli (Gai Lan), broccoli raab (rapini), brussels sprouts, cabbage, Chinese cabbage (bok choy), Chinese cabbage (napa), Chinese mustard cabbage (Gai Choy), cauliflower, cavalo broccolo, collards, kale, kohlrabi, mizuna, mustard greens, mustard spinach, rape greens, garlic, great-headed garlic, leck, chayote (fruit), Chinese waxgourd (Chinese preserving melon), citron melon, cucumber, gherkin, hyotan, cucuzza, hechima, Chinese okra, melon, balsam apple, balsam pear, bittermelon, Chinese cucumber, cantaloupe, casaba, crenshaw melon, golden pershaw melon, honeydew melon, honey ball melon, mango melon, Persian melon, pineapple melon, Santa Claus melon, snake melon, pumpkin, crookneck squash, scallop squash, straightneck squash, vegetable marrow, zucchini, butternut squash, calabaza, hubbard squash, acorn squash, spaghetti squash, watermelon, amaranth (Chinese spinach), arugula (roquette), beet greens, cardoon, celery, Chinese celery, celtuce, chaya, chervil, edible-leaved chrysanthemum, garland chrysanthemum, cress (garden and upland), dandelion, dock (sorrel), dokudami, endive (escarole), Florence fennel, gow kee, lettuce (head and leaf), orach, parsley, purslane (garden and winter), dadicchio (red chicory), rhubarb, spinach, New Zealand spinach, vine spinach, Swiss chard, watercress (upland), water spinach, eggplant, groundcherry (Physalis spp), bell pepper, chili pepper, pepper, pimento, sweet pepper, tomatillo, tomato, legume vegetables, grain lupin, sweet lupin, white lupin, field bean, kidney bean, lima bean, navy bean, pinto bean, runner bean, snap bean, tepary bean, wax bean, adzuki bean, asparagus bean, blackeyed pea, catjang, Chinese longbean, cowpea, crowder pea, moth bean, mung bean, rice bean, southern pea, urd bean, yardlong bean, broad bean (fava), chickpea (garbanzo), guar, jackbean, lablab bean, lentil, dwarf pea, edible-podded pea, English pea, field pea, garden pea, green pea, snowpea, sugar snap pea, pigeon pea, soybean (immature seed), sword bean, arracacha, arrowroot, Chinese artichoke, Jerusalem artichoke, beet (garden), burdock, canna, cassava (bitter and sweet), celeriac, Chayote (root), chervil (turnip-rooted), chicory, chufa (Cyperus esculentats), dasheen (taro), galangal, ginseng, horseradish, leren, kava (turnip-rooted), parsley (turnip-rooted), parsnip, radish, oriental radish, rutabaga, salsify, back salsify, Spanish salsify, skirret, sweet potato, tanier, turmeric, turnip, wasabi, yacon, yam bean, truc yam, and any combinations thereof. Each possibility represents a separate embodiment of the present invention.

In some embodiments, the treatment is for a plant or a plant part selected from the group consisting of a seed, seedling, plantlet, bulb, rhizome, tree, herb, shrub, creeper, vegetable, fruit, legume, mushroom, flower, and combinations thereof. The terms “plant” or a “plant part” as used herein, are meant to encompass any plant organ or tissue.

Treating a phytopathogenic bacterial disease in a plant, a plant part, or crop comprising contacting of the plant, the plant part, or the crop with an effective amount of the system according to the principles of the present invention.

The term “effective amount” as used herein refers to an amount of the delivery system that can reduce the number of gram-negative bacteria by at least 1-log reduction, by at least 2-log reduction, at least 3-log, or by at least 4-log reduction as compared to the number of viable bacteria prior to treatment/contact with the delivery system. Alternatively, the effective amount can reduce disease incidence and/or manifestations such as area of maceration, by at least about 5%, 10%, 20%, 30%, 40%, 50%, 60%, 70%, 80%, or by at least about 90%, or by 100% as compared to the disease incidence/manifestations prior to treatment/contact with the delivery system.

The plant can be contacted with the system by at least one of a foliar application, a soil application, application through irrigation systems, starter solutions, injection to soil, patch application, and aerial application. Each possibility represents a separate embodiment of the invention.

According to some embodiments, contacting the plant, plant part, or crop with the system comprises direct contact or indirect contact.

In some embodiments, the direct contact comprises foliar spraying by an aqueous composition comprising said system. In some embodiments, the carrier comprises LMP, gelatin, and trehalose. In exemplary embodiments, the carrier comprises κ-carrageenan and trehalose.

In some embodiments, the indirect contact of the plant with the system comprises administering the system to the soil in which said plant is planted. The surrounding conditions such as ion content and moisture percentage can regulate the release pattern. In some related embodiments, the system is in the form of granules.

According to some embodiments, the delivery system can be applied, directly or indirectly, once or as many times as required such as, for example, once a day, once every two days, once every three days, once a week for 1, 2, 3, 4, 5, or more times as required, so as to prevent or treat the phytopathogenic bacterial disease pathogen population to increase above disease-causing level. The delivery system can be applied 2 times a day, 3 times a day, or as required. According to an exemplary embodiment, the method comprises contacting a potato tuber with the delivery system of the invention comprising BALOs immobilized within a carrier comprising κ-carrageenan and trehalose once daily for 3-4 days, wherein the delivery system is applied to the soil adjacent the potato tuber.

According to further embodiments, the delivery system can be contacted with the plant, plant part, or crop before phytopathogen infection. Alternatively or additionally, the delivery system can be contacted with the plant, plant part, or crop after phytopathogen infection.

According to yet further embodiments, the delivery system can be contacted with the plant, plant part, or crop before, concomitantly with, or after treating said plant, plant part, or crop with any known treatment including, but not limited to, a physical treatment, e.g., hot water at about 50° C. to about 55° C. or dry hot air at about 50° C., and a chemical treatment, e.g., antibacterial agent such as an antibiotic agent, copper containing agents.

According to another aspect, there is provided a method for reducing bacterial load of an item in at least one of pharmaceutical industry, microbiological industry, medicinal industry, food industry, marine industry, paper industry, textile, plant nurseries, aquaculture, soil treatments, ship maintenance, water treatment facilities, sewage treatment facilities, drainage systems, and water distribution systems, the method comprising contacting the item with an effective amount of the delivery system according to the principles of the present invention, thereby reducing the bacterial load of said item.

According to some embodiments, the bacterial load is caused by, comprises, or consists of bacteria selected from the group consisting of gram-negative bacteria, gram-positive bacteria, antibiotic resistant bacteria, and any combination thereof. Without wishing to be bound by any theory or mechanism of action, it is contemplated that BALOs degrade the biofilm matrix of gram-positive bacteria, however such degradation does not involve predation upon the gram-positive bacteria.

According to some embodiments, the gram-negative bacteria are phytopathogenic bacteria. According to further embodiments, the gram-negative bacteria are pathogenic gram-negative bacteria. Pathogenic gram-negative bacteria include, but are not limited to, Escherichia coli, Vibrio spp., such as whiteleg shrimp-pathogenic vibrios, Vibrio cholerae, Vibrio parahaemolyticus, Vibrio vulnificus, Pseudomonas, Klebsiella, Plesiomonas shigelloides, Salmonella, Shigella, Yersinia enterocolitica, and Yersinia pseudotuberculosis. Each possibility represents a separate embodiment of the invention.

According to additional embodiments, the gram-positive bacteria are pathogenic gram-positive bacteria. Pathogenic gram-positive bacteria include, but are not limited to, Actinomyces, Bacillus, Listeria, Lactococcus, Staphylococcus, Streptococcus, Enterococcus, Mycobacterium, Corynebacterium, and Clostridium. According to further embodiments, the Staphylococcus or Streptococcus bacteria are selected from the group consisting of Staphylococcus aureus, Staphylococcus simulans, Streptococcus suis, Staphylococcus epidermidis, Streptococcus equi, Streptococcus equi zoo, Streptococcus agalactiae (GBS), Streptococcus pyogenes (GAS), Streptococcus sanguinis, Streptococcus gordonii, Streptococcus dysgalactiae, Group G Streptococcus, Group E Streptococcus, and Streptococcus pneumonia. Each possibility represents a separate embodiment of the invention.

According to yet further embodiments, the antibiotic-resistant bacteria include, but are not limited to, antibiotic-resistant Staphylococcus bacteria and antibiotic-resistant Streptococcus bacteria. According to further embodiments, the antibiotic-resistant Staphylococcus or Streptococcus bacteria are selected from the group consisting of methicillin-resistant Staphylococcus aureus (MRSA), vancomycin resistant Staphylococcus aureus (VRSA), daptomycin-resistant Staphylococcus aureus (DRSA), and linezolid-resistant Staphylococcus aureus (LRSA). Each possibility represents a separate embodiment of the invention.

According to some embodiments, the method of reducing bacterial load of an item comprises reducing or eradicating biofilm.

The term “biofilm” refers to a population of microorganisms, such as bacteria, growing on a surface, wherein the bacteria are encased in a matrix generally composed of polysaccharides, proteins, and nucleic acids. In this state, bacteria are less susceptible to both phagocytes and antibiotics. The term “biofilm” is further intended to include biological films that develop and persist at interfaces in aqueous environments.

The term “reducing biofilm” and “eradicating biofilm” are used herein interchangeably and are defined as the ability to degrade an existing or mature biofilm and/or to inhibit, prevent, or reduce the formation of a biofilm in vitro as well as in vivo and/or to kill bacteria embedded or associated with biofilms.

The terms “inhibiting biofilm formation”, “preventing biofilm formation” or “reducing biofilm formation” are used herein interchangeably and refer to the ability to avert or reduce the formation of a biofilm. According to some embodiments, preventing biofilm formation means inhibiting bacterial attachment to an outer surface of an item or to an inner surface of an item. Additionally or alternatively, preventing biofilm formation means killing the biofilm-forming bacteria.

The items which can be contacted with the delivery system include, but are not limited to, machinery and equipment for manufacturing or storage of pharmaceuticals; medicals devices or implants; contact lenses; machinery or equipment for food preparation and storage; tanks, pipes, filters, reservoirs; coolers; drainage systems, e.g., in hospitals, such as sinks, filters, pipes, and the like; water irrigation systems and any part thereof; boats and any part thereof.

The term “reducing bacterial load” as used herein refers to a reduction in the number of viable bacteria present on an outer surface of an item or on an inner surface of the item as a result of contacting it with the delivery system of the present invention as compared to the number of viable bacteria present on the surface of said item or within said item prior to contact with the delivery system. The number of viable bacteria can be reduced by at least 1-log reduction, at least 2-log reduction, at least 3-log reduction, or by at least 4-log reduction as compared to the number of viable bacteria prior to treatment/contact with the delivery system. The number of viable bacteria can be determined by any method known in the art, e.g., PFU, representing viable cells that can be counted by dilution plating in double-layered agar.

The delivery system can be contacted with the item before bacterial contamination and/or biofilm formation occurs, so as to prevent bacterial contamination and/or biofilm formation. Alternatively or additionally, the delivery system can be contacted with the item after bacterial contamination and/or biofilm formation.

### Methods of Preparation of the Delivery Systems

According to another aspect, there is provided a method for the preparation of a delivery system for the differential delivery of viable Bdellovibrio-and-like organisms (BALOs), wherein the delivery system comprises a carrier comprising a polysaccharide hydrocolloid and BALOs immobilized within the carrier, wherein the BALOs are in a physiological state of bdelloplasts, bdellocysts, and APs, which are present at a cell density of at least about 1.0×10⁸ PFU/(g carriers), the method comprises the following steps:
(i) admixing a suspension comprising BALOs with a hydrocolloid solution comprising the polysaccharide hydrocolloid to form a hydrocolloid composition comprising the BALOs; and
(ii) admixing the hydrocolloid composition comprising the BALOs of step (i) with a crosslinking solution to form the polysaccharide hydrocolloid in a wet gel form, thereby forming said delivery system in a wet form.

According to some embodiments, the method further comprises a step of drying the delivery system of step (ii), thereby forming said system in a dry form.

According to additional embodiments, drying the system is performed by at least one of an air drying, desiccant drying, vacuum drying, fluidized bed drying, spray-drying, sun drying, and freeze-drying. Each possibility represents a separate embodiment of the present invention. In some exemplary embodiments, drying the system is performed by vacuum drying. The advantages of vacuum drying compared to freeze-drying are that the product is not frozen, so energy consumption and the related economic and environmental impacts are reduced. Furthermore, potential freezing damage is avoided. The main issue with various drying techniques, however, lies in the survival of microorganisms during dehydration. This may be particularly acute with bacteria that do not form spores, i.e., all gram-negative taxa. After dehydration, the water availability within the carrier decreases until the immobilized cells reach a dormant state during which metabolism slows, sometimes to a complete standstill. It has been surprisingly found by the inventors of the present invention that survival rates of BALOs within the carrier after drying were higher for bdelloplasts compared to AP cells. Survival rates were further improved in the presence of osmoprotectants, wherein an osmoprotectant having high molecular weight increased the survival rate of bdelloplasts as compared to an osmoprotectant having low molecular weight.

According to further embodiments, the method further comprises a step of grinding, crushing, pulverizing and/or any process known in the art useful for reducing the Feret diameter of a granule, bead, capsule, or particle, thereby obtaining the dry system in the form of a powder.

According to some embodiments, admixing the hydrocolloid composition with the crosslinking solution is performed by at least one of dripping, spraying, emulsification, and inclusion in oil followed by chilling. In some exemplary embodiments, admixing the hydrocolloid composition with the crosslinking solution is performed by dripping the hydrocolloid composition into the crosslinking solution comprising a metal cation and/or a gelation-inducing hydrocolloid.

According to some embodiments, the hydrocolloid composition is an aqueous composition. The term “aqueous composition”, as used herein, refers to a solution or a mixture in which the solvent comprises from about 75% (w/w) to about 99.8% (w/w) water.

According to some embodiments, the polysaccharide hydrocolloid is present in the hydrocolloid solution in a weight percent ranging from about 0.25% to about 10% out of the total weight of the hydrocolloid solution. In further embodiments, the polysaccharide hydrocolloid is present in the hydrocolloid solution in a weight percent ranging from about 0.5% to about 5% out of the total weight of the solution. In yet further embodiments, the hydrocolloid is present in the hydrocolloid solution in a weight percent ranging from about 1% to about 3% out of the total weight of the solution.

According to some embodiments, the polysaccharide hydrocolloid is selected from the group consisting of κ-carrageenan, pectin, alginate, agar, gellan, carboxymethyl cellulose (CMC), furecellaran, xanthan gum, locust bean gum (LBG), konjac-mannan, chitosan, pullulan, curdlan and derivatives and combinations thereof. Each possibility represents a separate embodiment of the invention.

According to an exemplary embodiment, the polysaccharide hydrocolloid is κ-carrageenan.

According to some embodiments, the volumetric ratio between the hydrocolloid solution and the BALOs suspension, preferably the bdelloplasts suspension, ranges from about 1:3 to about 1:20, preferably from about 1:4 to about 1:9. According to an exemplary embodiment, the volumetric ratio between the hydrocolloid solution and the BALOs suspension is of about 1:9.

According to some embodiments, the hydrocolloid composition further comprises a protein hydrocolloid and/or a protein. According to further embodiments, protein hydrocolloids and proteins include, but are not limited to, gelatin, collagen, collagen hydrolysate, albumin, casein, whey protein, zein, soy protein, and any derivative or combination thereof. Without wishing to be bound by any mechanism of action, the addition of a protein hydrocolloid such as gelatin can modify the rate of degradation of the carrier by gelatinases secreted by the prey, thereby releasing the encapsulated BALOs at the desired release rate.

According to some embodiments, the hydrocolloid composition further comprises a water-absorbing agent. In some embodiments, the water-absorbing agent is selected from the group consisting of starch, polyvinylpyrrolidone (PVP), and derivatives and combinations thereof. Each possibility represents a separate embodiment of the invention.

In some embodiments, the polysaccharide hydrocolloid is LMP.

According to some embodiments, gelatin or the water-absorbing agent are present in the hydrocolloid solution in a weight percent ranging from about 0.25% to about 25% out of the total weight of the hydrocolloid solution. In further embodiments, gelatin or the water-absorbing agent are present in the hydrocolloid solution in a weight percent ranging from about 0.5% to about 10%, out of the total weight of the solution. In yet further embodiments, gelatin or the water-absorbing agent are present in the hydrocolloid solution in a weight percent ranging from about 1% to about 5% out of the total weight of the hydrocolloid solution.

According to some embodiments, the hydrocolloid composition further comprises at least one osmoprotectant. According to further embodiments, the at least one osmoprotectant is selected from the group consisting of trehalose, inositol, glycerol, arabitol, betaine, an amino acid, and combinations thereof. Each possibility represents a separate embodiment of the invention. In some embodiments, the at least one osmoprotectant is trehalose and/or glycerol.

In certain embodiments, the hydrocolloid is κ-carrageenan and the at least one osmoprotectant is trehalose.

According to some embodiments, the osmoprotectant is present in the hydrocolloid solution in a weight percent ranging from about 0.25% to about 15% out of the total weight of the hydrocolloid solution. In further embodiments, the osmoprotectant is present in the hydrocolloid solution in a weight percent ranging from about 0.5% to about 12.5% out of the total weight of the solution. In yet further embodiments, the osmoprotectant is present in the hydrocolloid solution in a weight percent ranging from about 1% to about 10% out of the total weight of the hydrocolloid solution.

According to some embodiments, the crosslinking solution and the hydrocolloid composition are contacted at a volumetric ratio of from about 10:1 to about 500:1. In further embodiments, the crosslinking solution and the hydrocolloid composition are contacted at a volumetric ratio of from about 30:1 to about 300:1. In yet further embodiments, the crosslinking solution and the hydrocolloid composition are contacted at a volumetric ratio of from about 50:1 to about 150:1. According to a certain embodiment, the crosslinking solution and the hydrocolloid composition are contacted at a volumetric ratio of about 100:1.

According to some embodiments, the crosslinking solution comprises a metal cation and/or a gelation-inducing agent. According to additional embodiments, the crosslinking solution comprises the metal cation and/or the gelation-inducing agent in a weight percent ranging from about 0.1% (w/w) to about 5% (w/w) out of the total weight of the crosslinking solution. In further embodiments, the crosslinking solution comprises the metal cation and/or the gelation-inducing agent in a weight percent ranging from about 0.2% (w/w) to about 4% (w/w) out of the total weight of the crosslinking solution. In yet further embodiments, the crosslinking solution comprises the metal cation and/or the gelation-inducing hydrocolloid in a weight percent ranging from about 0.3% (w/w) to about 1% (w/w) out of the total weight of the crosslinking solution.

In some embodiments, the metal cation is selected from the group consisting of a calcium (Ca²⁺), potassium (K⁺), barium (Ba²⁺), magnesium (Mg²⁺), aluminum (Al³⁺), zinc (Zn²⁺), lead (Pb²⁺), ferrous (Fc²⁺), strontium (Sr²⁺), copper cation (Cu²⁺), cadmium (Cd²⁺), nickel (Ni²⁺), cobalt (Co²⁺), and combinations thereof. Each possibility represents a separate embodiment of the invention.

In some exemplary embodiments, the metal cation is calcium (Ca²⁺). In certain such embodiments, the hydrocolloid is LMP.

In yet another exemplary embodiment, the metal cation is potassium (K⁺). In certain such embodiments, the hydrocolloid is κ-carrageenan.

In some embodiments, the gelation-inducing agent is a polycationic hydrocolloid and/or a polycationic compound selected from the group consisting of LBG, genipin, chitosan, polylysine, polyarginine, and derivatives and combinations thereof. Each possibility represents a separate embodiment of the invention.

According to exemplary embodiments, the method for the preparation of the delivery system of the present invention comprises the following steps:
(i) admixing a suspension comprising BALOs with a hydrocolloid solution comprising the polysaccharide hydrocolloid to form a hydrocolloid composition comprising the BALOs,
   wherein the polysaccharide hydrocolloid is κ-carrageenan or LMP present in the hydrocolloid solution in a weight percent ranging from about 0.25% to about 10%, preferably in a weight percent ranging from about 1% to about 3%, out of the total weight of the hydrocolloid solution;
   wherein the hydrocolloid solution further comprises trehalose in a weight percent ranging from about 0.25% to about 15%, preferably in a weight percent ranging from about 1% to about 10%, out of the total weight of the hydrocolloid solution;
   wherein the suspension of BALOs is added to the hydrocolloid solution in a volumetric ratio ranging from 1:100 to about 1:4, preferably in a volumetric ratio of 1:9; and
(ii) admixing the hydrocolloid composition comprising the BALOs of step (i) with a crosslinking solution to form the polysaccharide hydrocolloid in a wet gel form,
   wherein the crosslinking solution comprises a metal cation;
   wherein if the polysaccharide hydrocolloid is κ-carrageenan, the metal cation is potassium ion, and if the polysaccharide hydrocolloid is LMP, the metal cation is calcium ion; and
   wherein the metal cation is present in the crosslinking solution in a weight percent ranging from about 0.1% to about 5%, preferably in a weight percent ranging from about 0.3% to about 1%, out of the total weight of the crosslinking solution.

According to further exemplary embodiments, if the hydrocolloid solution being used in the method for the preparation of the delivery system comprises LMP, the hydrocolloid solution can further comprise gelatin in a weight percent ranging from about 0.25% to about 25%, preferably in a weight percent ranging from about 1% to about 5%, out of the total weight of the hydrocolloid solution.

According to some embodiments, the drying is performed at a temperature ranging from about 10° C. to 60° C. In further embodiments, the drying is performed at a temperature ranging from about 20° C. to 50° C. In yet further embodiments, the drying is performed at a temperature ranging from about 30° C. to 40° C.

According to some embodiments, the vacuum-drying is performed at a pressure ranging from 10 mbar to 1000 mbar. In further embodiments, the vacuum-drying is performed at a pressure ranging from 50 mbar to 800 mbar. In yet further embodiments, the vacuum-drying is performed at a pressure ranging from 100 mbar to 500 mbar.

According to some embodiments, following the drying the system comprises at least about 1.0×10⁸ (PFU/g carrier) BALOs or preferably bdelloplasts. According to further embodiments, following the drying the system comprises at least about 5.0×10⁸ (PFU/g carrier), about 1.0×10⁹ (PFU/g carrier), or about 5.0×10⁹ (PFU/g carrier) BALOs, or preferably bdelloplasts.

As used herein and in the appended claims the singular forms “a”, “an,” and “the” include plural references unless the content clearly dictates otherwise. Thus, for example, reference to “carrier” includes a plurality of such carriers and equivalents thereof known to those skilled in the art, and so forth. It should be noted that the term “and” or the term “or” is generally employed in its sense including “and/or” unless the content clearly dictates otherwise.

As used herein, the term “about”, when referring to a measurable value such as an amount, a temporal duration, and the like, is meant to encompass variations of +/−20%, preferably +/−10%, more preferably +/−5%, and even more preferably +/−1% from the specified value, as such variations are appropriate to perform the disclosed methods.

The terms “plurality” or “multiple”, as used herein, mean two or more.

Numerical ranges disclosed throughout the specification and claims such as amounts, ratios, and time periods, include any integer and numerical fraction in between.

It is to be understood that each possibility disclosed throughout the specification represents a separate embodiment of the invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Example 1

### Bacterial Strains, Media and Growth Conditions

Bdellovibrio-and-like organisms (BALOs) used as predatory bacteria were Bdellovibrio bacteriovorus HD100 and HD100-Td-tomato, containing plasmid pMQ414 and constitutively expressing the fluorescent td-Tomato protein. Lab stock of B. bacteriovorus was kept at −80° C.

Escherichia coli ML35, Pectobacterium carotovorum subsp. carotovorum WPP14, Pectobacterium carotovorum subsp. brasiliense (Pcb) and Dickeya solani were used as prey bacteria.

The prey bacteria P. carotovorum spp. and Dickeya solani were grown in LB medium at 28° C. under shaking at 250 rpm. E. coli was grown at 37° C. All were started from single colonies originating from laboratory stocks kept at −80° C. Overnight cultures were centrifuged at 4500 g for 10 min at 4° C., and the cell pellet was washed and resuspended in HEPES buffer pH 7.8 amended with 2 mM CaCl₂) and 3 mM MgCl₂·6H2O (aHEPES), to a final optical density (OD₆₀₀) of 10 for the P. carotovorum spp., Dickeya solani, and E. coli. The E. coli suspension was further concentrated to yield ˜10¹¹ colony forming units (CFU)/ml. The stock suspensions were stored at 4° C. until use, for up to two weeks.

To initiate predatory co-cultures, a bacteriological loopful from the frozen stock of the bacterial predator B. bacteriovorus HD100 or HD100-Td-tomato was transferred to a flask containing 1.5 ml of an OD₆₀₀=10 E. coli suspension diluted in 15 mL of aHEPES. The co-cultures were incubated at 28° C. under shaking at 250 rpm until the culture's turbidity reached below OD₆₀₀=0.1. Then the suspension was filtered through a 0.45 μm filter (Sartorius). Thereafter, 5 ml of the filtered predatory cells from the starter culture were inoculated into 40 ml of amended HEPES containing 5 ml of an OD₆₀₀=10 E. coli suspension and shaken at 250 rpm at 28° C. until the suspension's turbidity decreased. After most of the prey cells were lysed, the suspension was filtered through a 0.45 μm filter yielding ˜10⁹ plaque-forming units (PFU)/ml of predatory AP cells. The filtrate was stored at 4° C. until used for up to one day. To further concentrate the filtered predatory cells, the suspension (50 ml) was centrifugated at 9000 rpm for 20 min at 10° C. and the cell pellet was resuspended in 0.5 ml of aHEPES to a final concentration of ˜10¹¹ PFU/ml of predatory AP cells. To create large amounts of bdelloplasts, synchronization of the predator growth was performed by addition of 250 μl of the concentrated E. coli suspension (10¹¹ CFU/ml) to 500 μl of the concentrated AP cells suspension (10¹¹ PFU/ml) and incubation for about 1 hour at 28° C. under shaking at 250 rpm. Bdelloplasts' formation was verified by phase microscopy.

Prey and predator cultures were routinely counted by dilution plating as CFU per ml and PFU per ml, respectively. The latter plating was performed in double-layered agar.

### Example 2

### Preparation and Characterization of κ-Carrageenan Dried Gel Carriers

### Preparation of κ-Carrageenan Dried Gel Carriers

1.5% (w/w) κ-carrageenan dispersions were prepared by the addition of κ-carrageenan powder (Sigma, USA) to distilled water (DW). The mixture was then brought to a boil to complete gum dissolution. The solution (at 40° C.) was dripped into a 1% (w/w) solution of KCl (Biolab Chemicals, Israel) at a volumetric ratio of 1:100 and stirred for 20 min. The carriers were then dried in a vacuum oven (Binder, Germany) at 37° C. and 400 mbar.

### Swelling Ratio Measurement of κ-Carrageenan Dried Gel Carriers

Swelling measurements of κ-carrageenan dried gel carriers were performed by introducing the carriers into DW or 1% or 2% KCl solutions. Periodically, the carriers were removed from the external solutions, wiped with filter paper, and weighed (+0.001 g) on an analytical balance (Precisa, Switzerland). The test was ended when the carriers started to disintegrate (in DW) or reached a constant weight (in 1 and 2% KCl solutions).

The swelling ratio by weight was calculated as:

$$Swelling ratio = \frac{W ( final )}{W ( initial )}$$

where W (initial) is the initial specimen weight (g) and W (final) is the final specimen weight (g).

### Potassium Concentration Measurements

For determining the potassium concentration (mg/L) released from the dried K-carrageenan carriers, samples from the external solution (10 ml DW) were collected at different times (a sample at each time was taken from a different test tube) and tested by inductively coupled plasma atomic emission spectroscopy (ICP-AES) (Spectroflame ICP, Spectro Analytical Instruments, Germany). A negligible potassium concentration was found in DW (blank sample).

### Preparation of κ-Carrageenan Carriers for the Encapsulation of BALOs

κ-Carrageenan (1.67%; w/w) dispersions were prepared by addition of κ-carrageenan powder (Sigma, USA) to DW. Different osmoprotectants were then added to the κ-carrageenan dispersion to a final concentration (w/w) of 10% glycerol (Sigma, USA), 5% trehalose (Sigma, USA), 5% betaine (Sigma, USA) or 5% inositol (Sigma, USA). The mixture was then brought to boil to complete gum dissolution. Bdelloplasts suspension was then added at a 1:9 volumetric ratio to a κ-carrageenan-based solution at 40° C. This final mixture was dripped into a 1% (w/w) (or else mentioned) sterile solution of KCl (Biolab Chemicals, Israel) at a volumetric ratio of 1:100 and stirred for 20 min. The obtained crosslinked spherical carriers, with an average diameter of about 4 mm, were then dried in a vacuum oven (Binder, Germany) at 37° C. and 400 mbar to obtain carriers with a diameter of about 1 mm.

### Encapsulation Efficiency

The encapsulation efficiency (EE) represents a combined measure of the efficacy of entrapment and survival of viable cells during the encapsulation procedure, calculated according to the following equation (Gebara et al., 2013):

$$EE ( \% ) = ( N / {N}_{0} ) \times 100$$

where EE is the encapsulation efficiency, expressed in percentage, N is the number of cells released from the carriers (CFU/g of carriers), and No is the number of cells in the hydrocolloid solution (CFU/g of suspension).

### Bacterial Enumeration in the Carriers

For quantitative measurements of cell viability, the carriers were solubilized in order to release the encapsulated Bdelloplasts. To this end, wet or dried gel carriers were immersed in sterile 2% sodium hexametaphosphate (SHMP) or sterile DW, respectively. The mixtures were then shaken until all carriers were dissolved. PFUs, representing viable cells were counted by dilution plating in double-layered agar.

### Scanning Electron Microscopy (SEM)

The different κ-carrageenan-based dried carriers were glued with double-sided carbon sticky tape to an aluminum stub and coated with 3 nm Au—Pd in a Q150TES coater (Quorum Technologies Ltd., Lewes, England). To study the characteristic carrier structure, a Jeol IT-100 SEM (Tokyo, Japan) was used at 20 kV. To visualize the bacteria that reside within the carriers, a Jeol JSM-7800F high-resolution SEM (Tokyo, Japan) was used at 2 kV.

### Predation Dynamics by Released Encapsulated Predators

Predation by encapsulated B. bacteriovorus HD100-Td-tomato in κ-carrageenan carriers was tracked by monitoring the predator growth using fluorescence and prey decay using OD in a plate reader incubated at 28° C. (Tecan Spark 10M, Switzerland). The fluorescence detectable threshold for the lower and upper levels corresponded to ˜4×10⁷ and ˜10⁹ PFU/ml B. bacteriovorus HD100, respectively. Five hundred μl of dissolved carriers entrapping predators at a ratio of 1 dried carrier in 1 ml DW, 100 μl of the prey (E. coli and Pectobacterium carotovorum, OD₆₀₀=10), and 400 μl amended HEPES buffer were added to an Eppendorf tube and mixed. For each treatment, 200 μl were pipetted onto wells of clear black flat-bottom 96-well plates (Greiner-Bio One, Germany) in three technical replicates, in two independent experiments.

### Potato Slice Assay

The red-skinned Desiree potato cultivar was sourced from local supermarkets and used in all experiments. Potatoes were surface sterilized by dipping for 1 min in a 1% HCl solution, washed twice with sterile DW, and left to dry in a laminar flow hood. After potato surfaces were dried, potatoes were washed with 70% ethanol and again dried on sterile petri dishes. Slices of about 0.5 cm width were cut using a sterilized knife and were transferred using sterile tweezers to Petri dishes containing sterile 70 mm-diameter filter paper (Whatman 1) dampened with 330 μl of sterile DW.

P. carotovorum subsp. Brasiliense was grown as above, subcultured (˜100 μl) in fresh LB to an OD₆₀₀ of ˜0.2 and washed twice in aHEPES buffer; the OD₆₀₀ was adjusted to 0.2, then diluted to yield ˜10⁶ CFU/ml. Potato slices were inoculated with 10 μl drop of the pathogen culture, and with 10 μl or 20 μl drops of the different tested treatments; both were applied to the center of the slice. Tested treatments included two different concentrations (diluted or concentrated) of dissolved κ-carrageenan-trehalose carriers cross-linked by 0.5% KCl and encapsulating bdelloplasts. For diluted and concentrated liquids, the ratios were of 1 dried carrier in 0.5 ml DW and 1 dried carrier in 0.15 ml DW, respectively. Liquids from dissolved carriers were applied 60 min ahead of P. carotovorum subsp. brasiliense. The potato slices were incubated at 28° C. for 48 h. All experiments were repeated three times, with 6 replicates per treatment. After incubation, slices were photographed using a ruler for scale. The maceration area was calculated using the ImageJ image processing program.

### Biofilm Assays

Microtiter wells were inoculated (200 μl per well) from 18 h Pseudomonas fluorescens and E-coli ZK2686 LB-grown cultures (at 28° C. and 37° C., respectively) diluted 1:100 in LB. Cells were grown for 48 h at 28° C. (preformed biofilm). To assess predation dynamics of encapsulated B. bacteriovorus on these biofilms, the preformed biofilms were grown as described above, washed three times with DDNB in order to remove any planktonic cells, and 200 μl of dissolved or ground carriers in DDNB (in a ratio of 1 carrier to 1 ml) were added to the wells. As a control, 200 μl of DDNB was added to the wells. The microtiter dish was incubated at 28° C. for 72 h before it was stained with crystal violet (CV) and quantified as described previously (O'Toole, G. A., and R. Kolter. 1998. Flagellar and twitching motility are necessary for Pseudomonas aeruginosa biofilm development. Mol. Microbiol. 30:295-304; Kadouri, D., & O'Toole, G. A. 2005. Susceptibility of biofilms to Bdellovibrio bacteriovorus attack. Applied and Environmental Microbiology, 71 (7), 4044-4051). by using a plate reader (Tecan Spark 10M, Switzerland) at 600 nm.

### Example 3

### Properties of κ-Carrageenan Dried Gel Carriers

For fast predator release from the carriers, several hydrocolloid gelling agents were tested. In preliminary experiments, κ-carrageenan-based carriers cross-linked by KCl were found to meet these requirements.

Comparing the disintegration rate in distilled water (DW) of κ-carrageenan wet or dried gel carriers, it was found that the dry carriers solubilized faster.

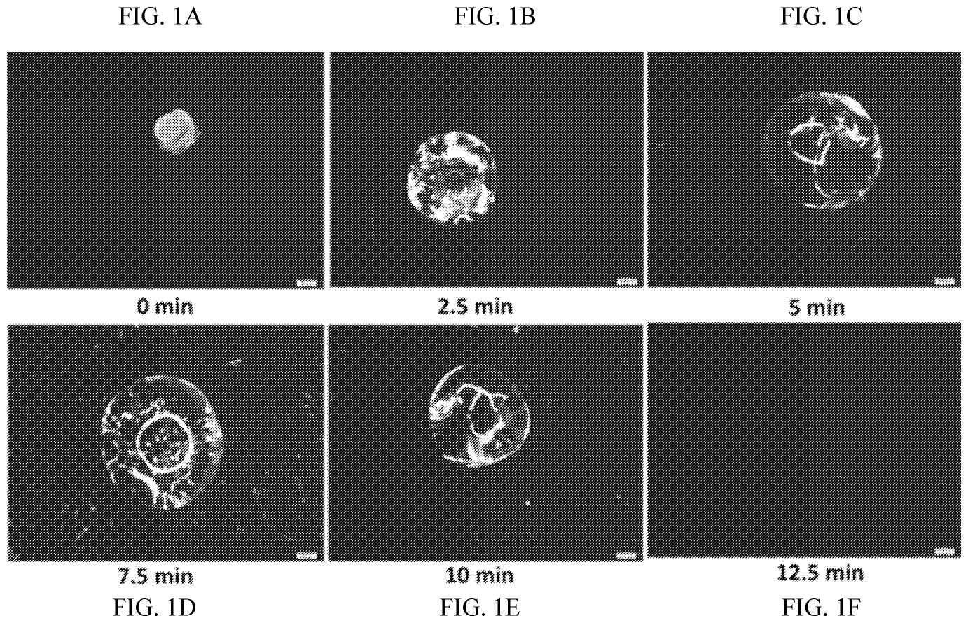

FIG. 1 demonstrates the characteristic swelling process and disintegration of a dried K-carrageenan carrier (350 μm diameter) when immersed in excess DW. The carriers were gradually swelled until complete disintegration after 12.5 min.

The effect of carrier size and the volume of water in the external solution on the swelling and disintegration of κ-carrageenan dry carriers was examined. The weight ratio between the carrier/s to the water was maintained for each water volume by placing 2 halves of a carrier together. Table 1 shows that the carriers swelled up to 30-34 times their initial weight, and for smaller carriers-maximum swelling before disintegration occurred earlier than for the larger carriers. Furthermore, the larger the water volume and the smaller the size of the carrier were, the disintegration occurred faster

TABLE 1
Parameters characterizing disintegration of κ-carrageenan
dried carriers in DW at 25° C.

| | Elapsed time for | Maximum swelling | |
|---|---|---|---|
| | maximum swelling | ratio (by weight) | Time of |
| | before disinte- | before disinte- | disinte- |
| | gration | gration | gration |
| Sample | (min) | (—) | (min) |
| 1 carrier in 10 ml | 30 | 34.4^{a} ± 1.6 | 60 |
| 1 carrier in 1 ml | 30 | 30.3^{b} ± 2.1 | 75 |
| 2 halves of a | 15 | 33.4^{ab} ± 4.6 | 45 |
| carrier in 10 ml | | | |
| 2 halves of a | 15 | 32.1^{ab} ± 2.6 | 60 |
| carrier in 1 ml | | | |

Superscript letters indicate significant differences (Student's t-test, P < 0.05).

The presented results are averages of 6 repetitions±SD from two different batches. Without wishing to be bound by any theory or mechanism of action, it is contemplated that the κ-carrageenan carriers disintegrated due to a loss of potassium ions which are the main cross-linking agents.

The swelling and disintegration processes were further examined when κ-carrageenan dried carriers were immersed in KCl solution, the main crosslinking agent, at different concentrations. As a result, the carriers did not disintegrate but swelled. Table 2 shows the higher the potassium concentration in the external solution was, the less the carriers swelled

TABLE 2
Equilibrium swelling ratio values of κ-carrageenan dried
carriers immersed in 10 ml KCl solutions at 25° C.

| Sample | Swelling ratio (—) |
|---|---|
| Carrier in 1% KCl | 8.3^{a} ± 0.8 |
| Carrier in 2% KCl | 5.2^{b} ± 0.3 |

Superscript letters indicate significant differences(Student's t-test, P < 0.05). The presented results are averages of 6 repetitions ± SD from two different batches.

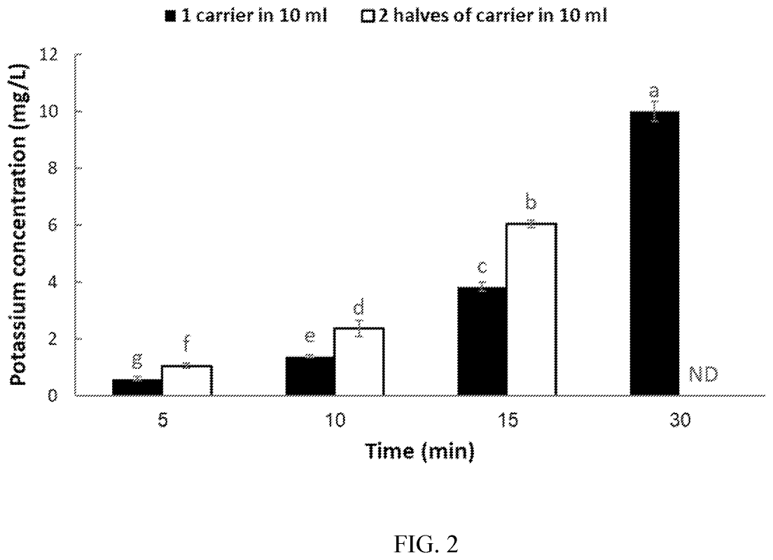

The concentration of potassium ions, which were released from the κ-carrageenan carriers before disintegration was measured as a function of time in an external solution of 10 ml DW. It was found that potassium concentration in the external solution increased with time, and the smaller the carriers, the higher and faster the increase. The total surface area of the 2 halved carriers is ˜1.5 times fold the surface of 1 intact carrier (assuming sphere shape), corresponding to the ratio in potassium concentration measured at each time point between the different sized carriers. The results are presented in FIG. 2.

### Example 4

### Encapsulation of BALOs in κ-Carrageenan Carriers and Survival Under Vacuum-Drying

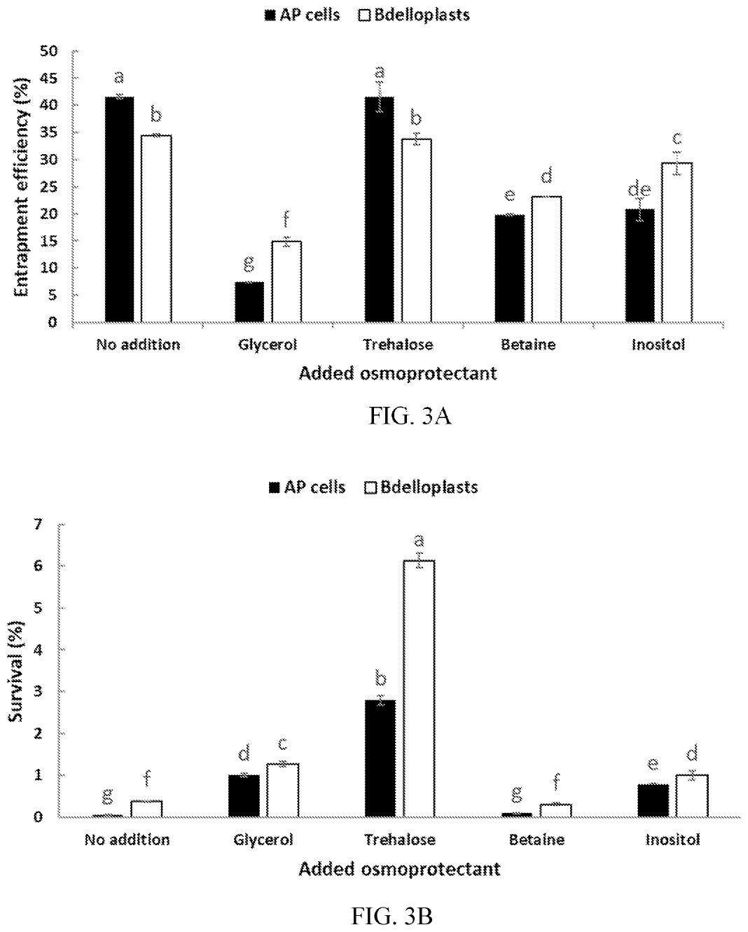

Encapsulation experiments of B. bacteriovorus HD100 in two different physiological states, AP cells or bdelloplasts, were performed. The effect of different osmoprotectants (glycerol, trehalose, betaine, and inositol) on the entrapment efficiency and survival rates of the predator bacterial cells is demonstrated in FIGS. 3A and 3B, respectively. The entrapment efficiency of AP cells and bdelloplasts varied from 7.4% with glycerol, to 41% with trehalose or without any addition, and appeared to decrease as the molecular weight of the added osmoprotectant was lower (FIG. 3A). Survival rates after drying were higher for bdelloplasts compared to AP cells (FIG. 3B). AP cells with no addition or with trehalose addition demonstrated survival rates of 0.05% and 2.8%, respectively, while bdelloplasts with betaine addition or with trehalose addition demonstrated survival rates of 0.3% to 6.1%, respectively (FIG. 3B).

Table 3 shows that encapsulated bdelloplasts with trehalose yielded the highest number of predators after drying (5.4×10⁹ PFU/g carriers), while the treatments without added osmoprotectants or with betaine fared the worst (Table 3). Regarding the percentage of survival after drying, a clear advantage of bdelloplasts over AP cells was observed (Table 3).

TABLE 3
The effect of predator cell type and added osmoprotectant
on encapsulated B. bacteriovorus HD100 density,
before and after vacuum-drying

| | | Before vacuum- | After vacuum- |
|---|---|---|---|
| Added | Physiological | drying (PFU/g | drying (PFU/g |
| Osmoprotectant | state | carriers) | carriers) |
| No Addition | AP | 9.3 × 10⁹ | 1.4 × 10⁸ |
| | Bdelloplasts | 3.9 × 10⁹ | 3.7 × 10⁸ |
| Glycerol | AP | 1.6 × 10⁹ | 3.8 × 10⁶ |
| | Bdelloplasts | 5.0 × 10⁹ | 1.4 × 10⁹ |
| Trehalose | AP | 6.7 × 10⁹ | 4.3 × 10⁹ |
| | Bdelloplasts | 3.8 × 10⁹ | 5.4 × 10⁹ |
| Betaine | AP | 3.5 × 10⁹ | 7.1 × 10⁷ |
| | Bdelloplasts | 2.3 × 10⁹ | 1.6 × 10⁸ |

Results represent the average of two independent experiments with three technical replicates for each treatment. In every test, five carriers were evaluated.

### Example 5

### The Effect of κ-Carrageenan Carriers Composition on their Structure

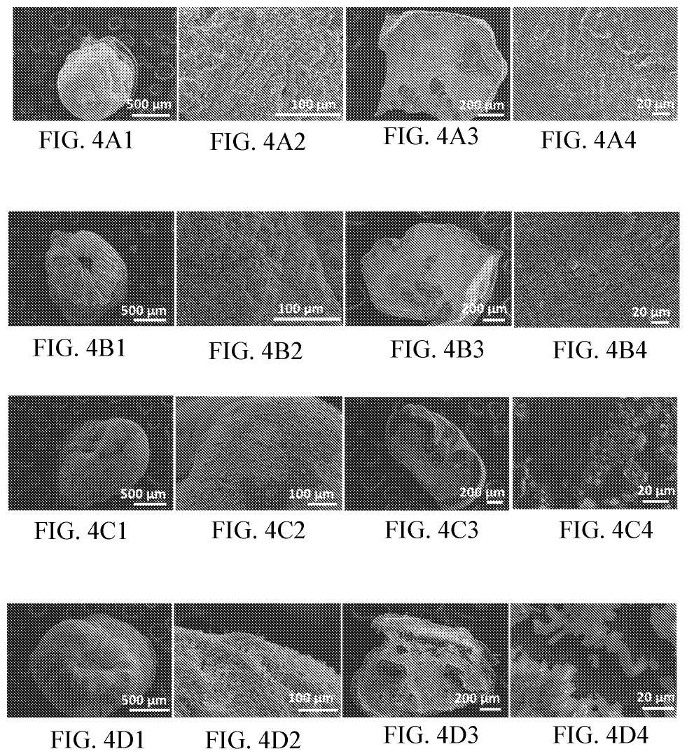

FIGS. 4A1-4D4 display SEM images of typical structures observed in various dried k-carrageenan carrier samples. KCl crystals were detected both on the surface as well as on the inner side of the carriers, presenting different crystallization patterns for each carrier sample depending on the added osmoprotectant. Dense zones (dark color in the halved carriers) and more porosive zones, usually near the carrier walls, were observed. For carriers with glycerol, a porous sponge-like structure on the outer side was distinguished.

### Example 6

### Predation Dynamics of Encapsulated B. bacteriovorus in κ-Carrageenan Carriers on Soft Rot Bacteria

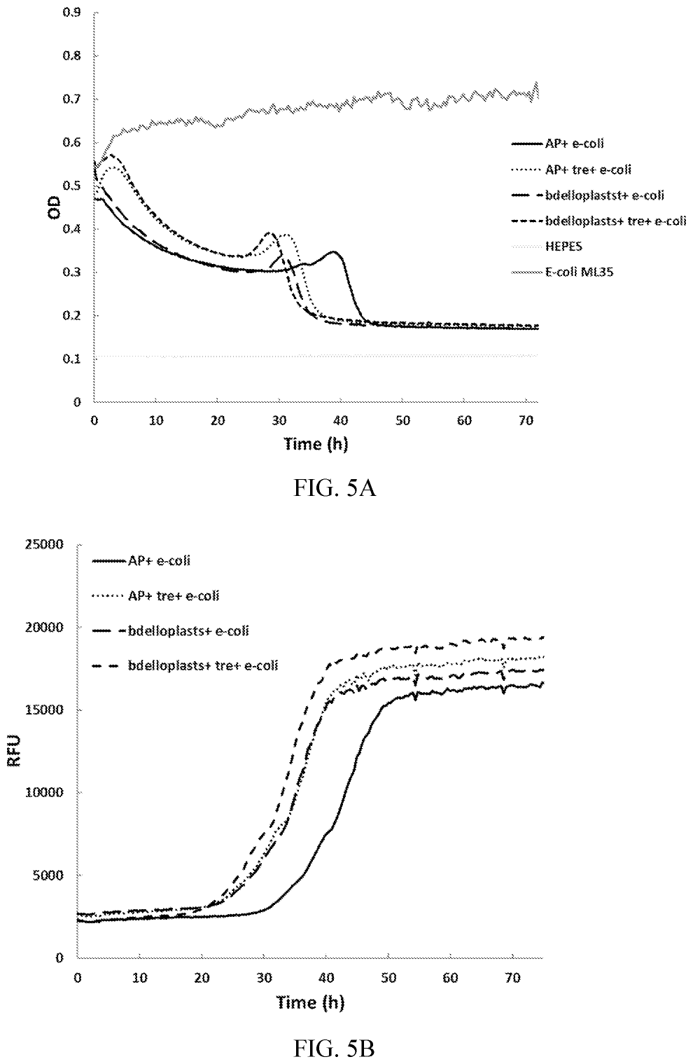

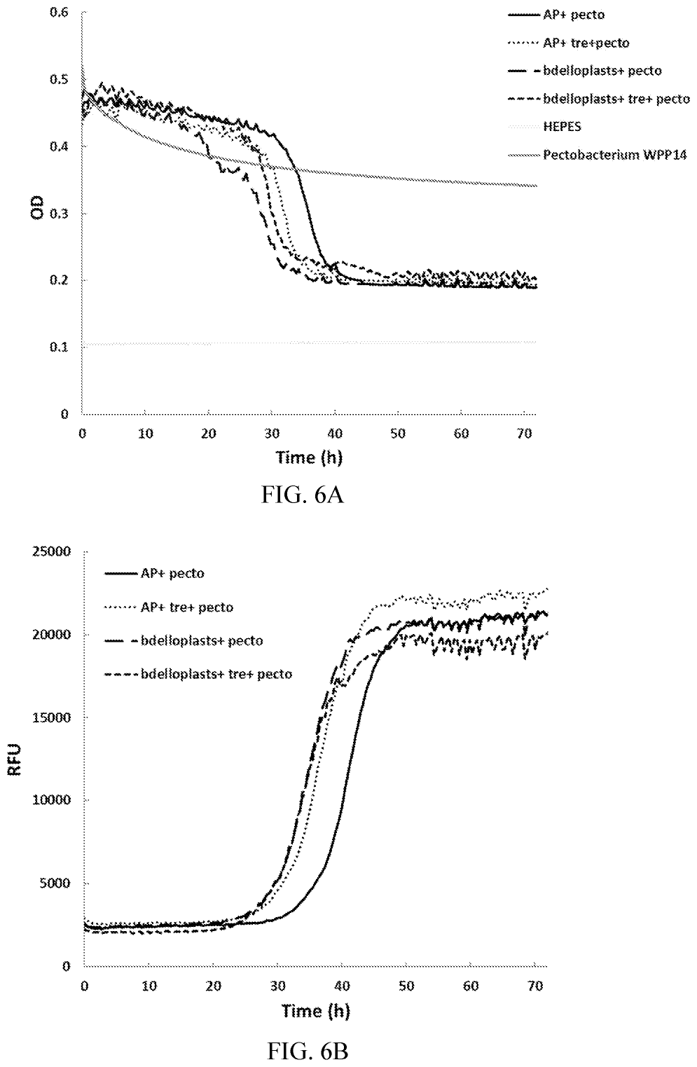

To test the predation dynamics of the predator encapsulated in κ-carrageenan carriers, B. bacteriovorus HD100 expressing the Td-tomato fluorescent protein were used. Predation of E. coli (a “standard” laboratory prey) and P. carotovorum by encapsulated AP cells or bdelloplasts predators, when released from carriers with or without trehalose, was examined (FIGS. 5A-5B and FIGS. 6A-6B). FIGS. 5A and 6A show a decrease in prey concentration in all treatments tested, with encapsulated bdelloplasts showing better performance as compared to AP cells. Carriers with bdelloplasts and trehalose preyed better on E-coli (FIG. 5A), while carriers with bdelloplasts without additives showed slightly faster predation on P. carotovorum (FIG. 6B). Predation by carriers with AP cells without additives lagged the rest of the treatments with ˜ 10 h delay (FIGS. 5A and 6A).

FIGS. 5B and 6B show an increase in predatory populations in all treatments exposed to prey. A higher increase in the concentration of bdelloplasts, released from carriers containing trehalose, was obtained when E. coli constituted the prey population (FIG. 5B), while for P. carotovorum prey population, a higher increase in concentration was obtained for AP cells released from carriers.

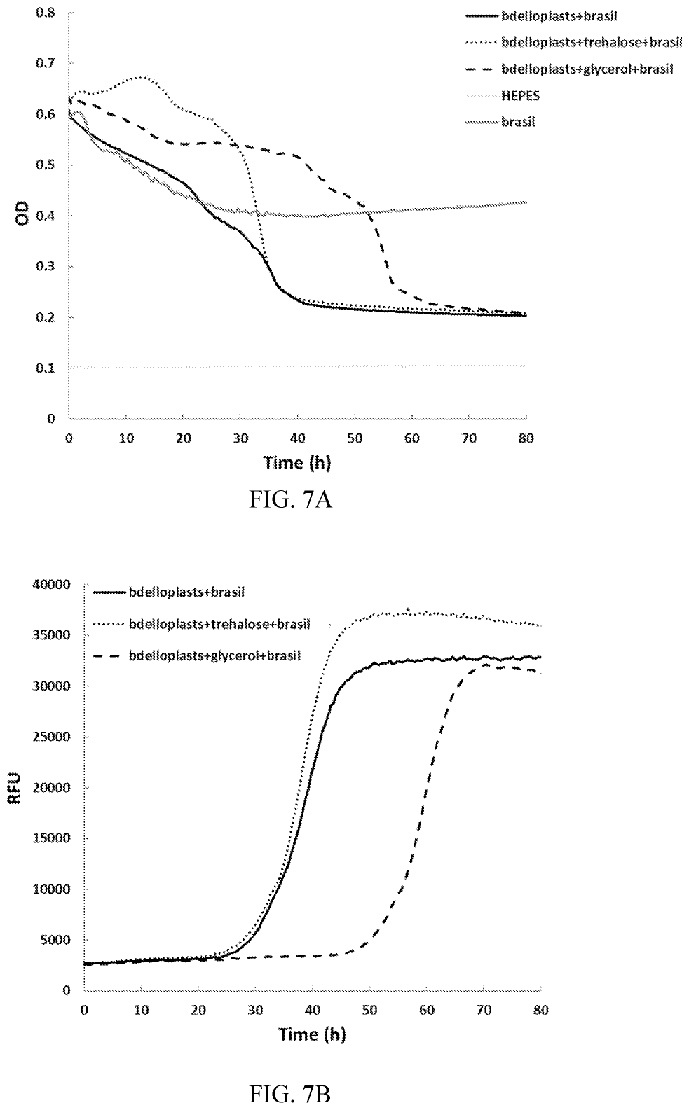

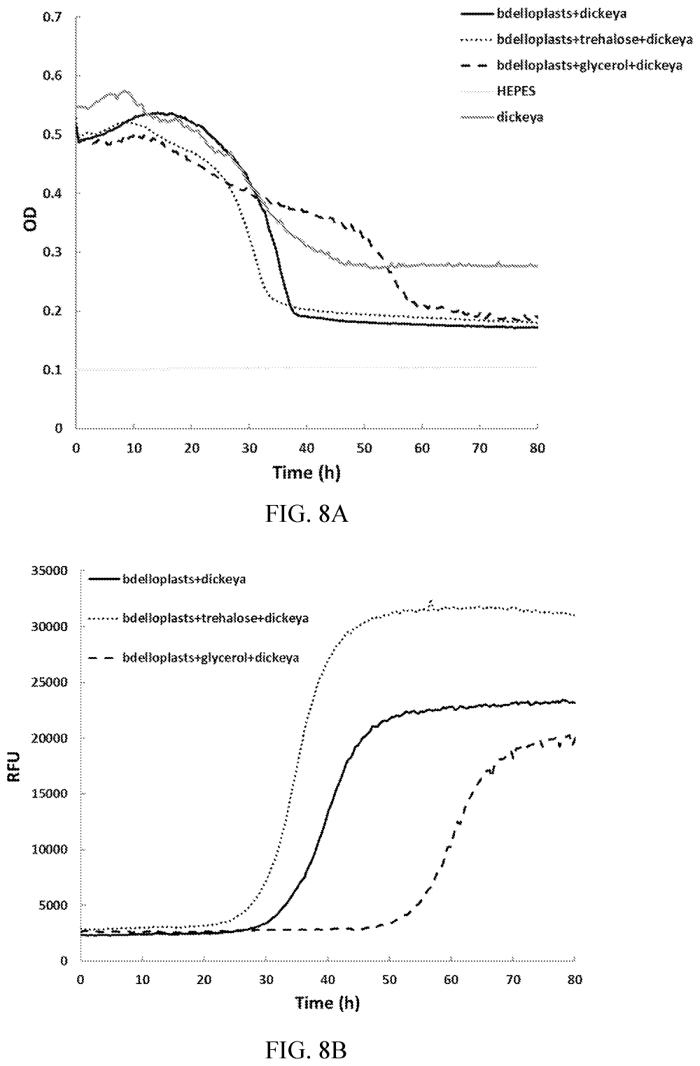

Carriers encapsulating bdelloplasts and including trehalose, glycerol or no additives were tested against two additional soft-rot bacteria: P. brasiliense (FIGS. 7A-7B) and Dickeya solani (FIGS. 8A-8B). The addition of glycerol resulted in much slower predation than the other treatments displaying a ˜25 h delay (FIGS. 7A and 8A). Predation dynamics on Pectobacterium was not different between the carriers with or without additives (FIG. 7A) and for the Dickeya a slight advantage for the carriers with trehalose was observed (FIG. 8A). Higher plateau RFU values were obtained for carriers with trehalose, as indicated by the increased numbers of predators after predation (FIGS. 7B and 8B).

### Example 7

### Effect of κ-Carrageenan Carrier Preparation Conditions on Predator Survival

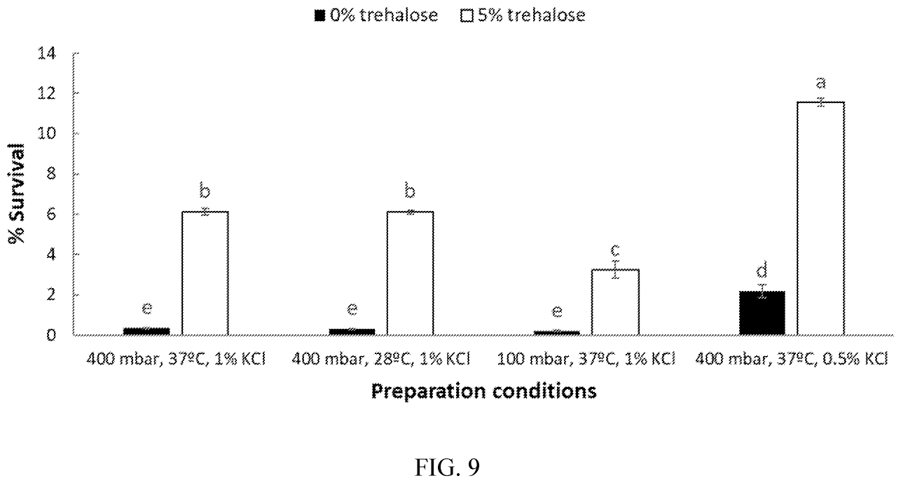

To further improve the survival of the predators, different drying conditions (vacuum and temperature) and cross-linker concentrations were examined. These experiments were conducted with the carriers encapsulating bdelloplasts with or without trehalose, as these previously yielded the best outcomes in terms of survival and predation. The results of these experiments are presented in FIG. 9. No difference in predator survival was observed between 37° C. and 28° C. Increasing vacuum from 400 to 100 mbar resulted in a small reduction or no change in survival. Decreasing the cross-linking agent concentration from 1 to 0.5% KCl significantly increased survival by about 2-fold, reaching 3.2×10⁹ PFU/g carriers when no trehalose was added. In its presence, survival increased to ˜12% or 1.3×10¹⁰ PFU/g carriers.

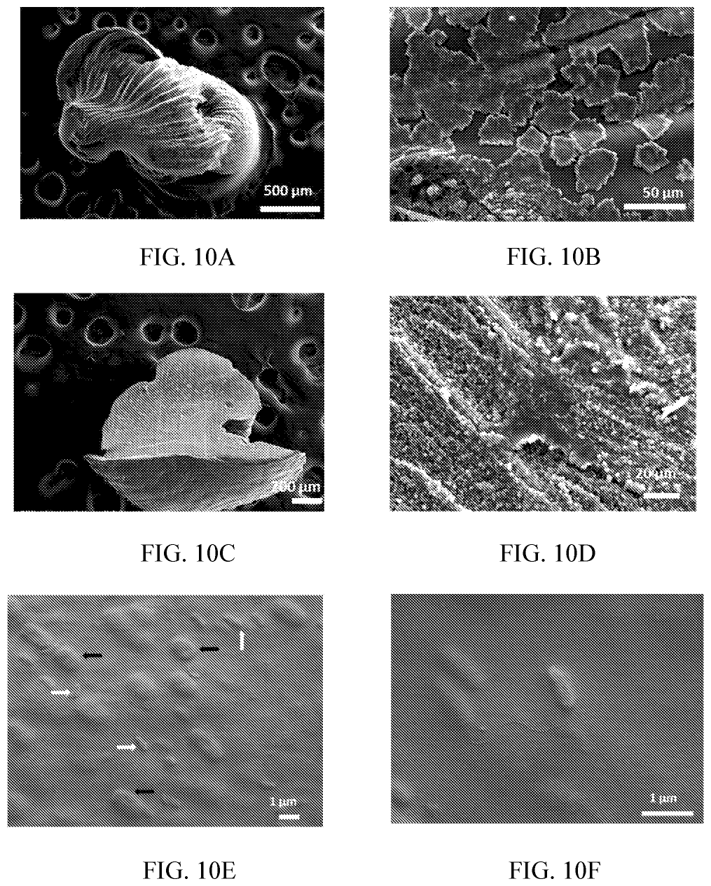

FIGS. 10A-10F illustrate SEM images of κ-carrageenan-trehalose carriers encapsulating predators prepared with 0.5% KCl. “Flakes” containing salts could be seen on the outer surface of the carriers (FIG. 10B). Crystals were also observed in the inner content of the carrier although they were smaller and of different configurations (FIG. 10D). Overall, fewer salts were detected compared to FIG. 4, where a higher KCl concentration was used. High-resolution SEM micrographs show a mixed population of AP cells and bdelloplasts, embedded within the matrix (FIGS. 10E and 10F). The HR-SEM micrographs show the B. bacteriovorus kept integrity both as bdelloplasts and as AP cells following vacuum-drying the κ-carrageenan-trehalose carriers.

### Example 8

### κ-Carrageenan Carrier Effectiveness in Preventing Disease in Potato Slices

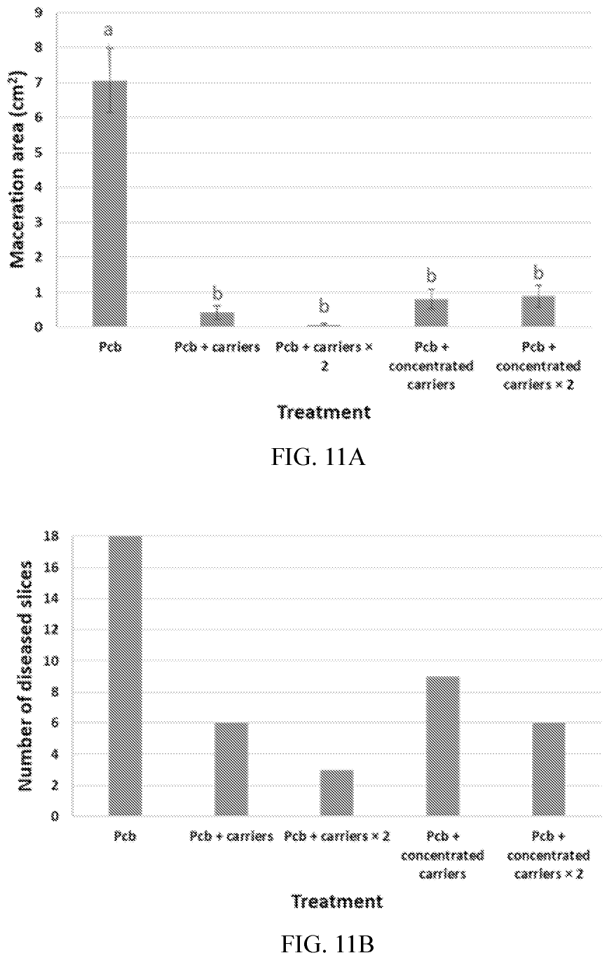

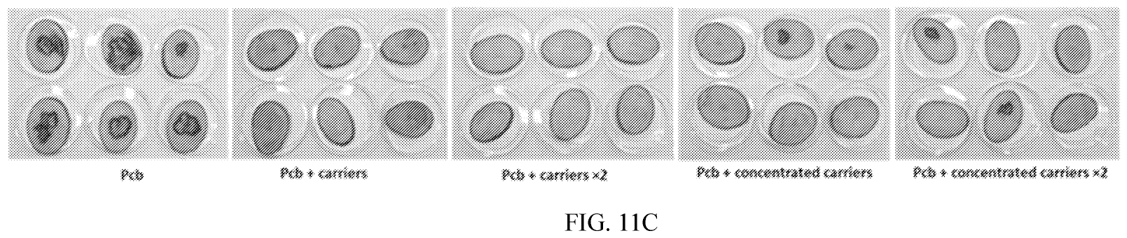

Potato slice assays were used to test the ability of the predators encapsulated in carrageenan-trehalose carriers to prevent soft rot Enterobacteria (SRE) maceration in situ. The carriers were dissolved in water and the suspension was applied 60 min before application of the highly virulent P. Brasiliense (Pcb) pathogen. The addition of diluted or concentrated suspensions obtained from dissolved carriers and the application of two different volumes of each, significantly reduced the maceration area (by ˜87-99%) (FIG. 11A) and the number of diseased slices (FIG. 11B) as compared to the P. brasiliense control. The lowest disease incidence was obtained in the treatment using the largest volume of the diluted suspension (FIG. 11B) as can be seen in the photographs of the potato slices as well (FIG. 11C).

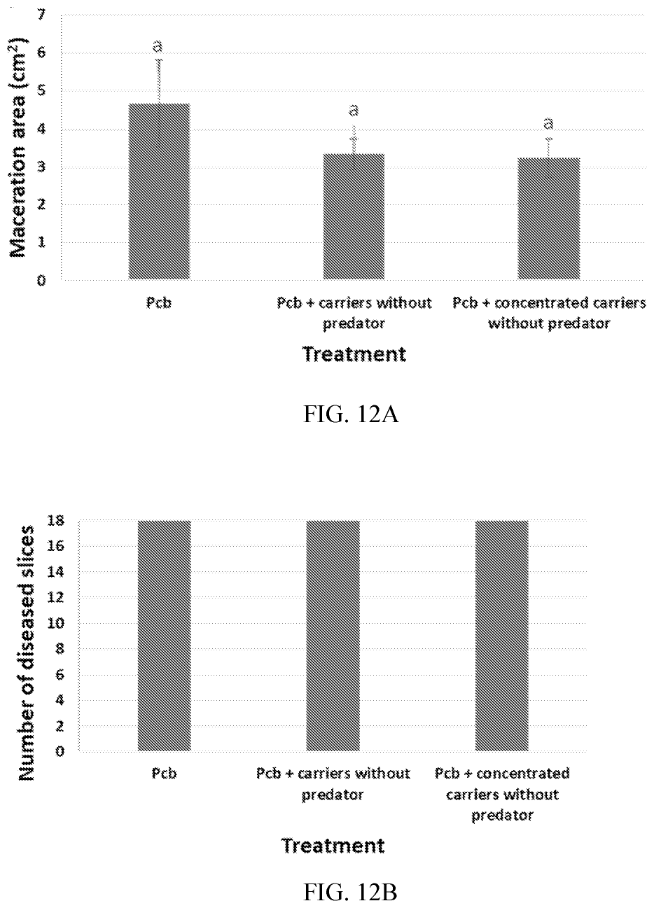

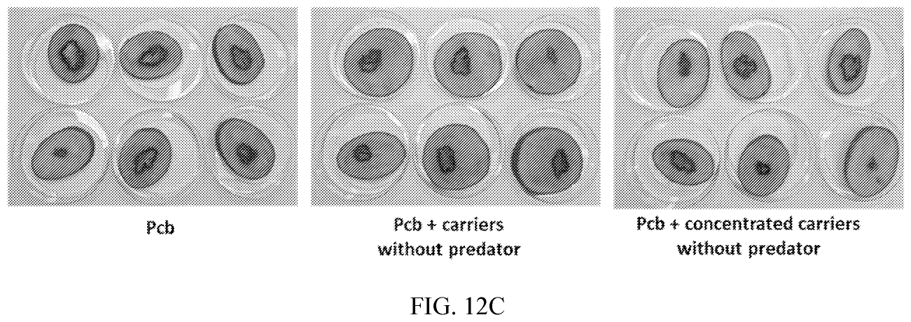

In order to confirm that the observed effect was not the result of indirect interactions of the carrier components such as elicitation of plant defenses, carriers without predators were tested. FIG. 12A-12C show that the treatments were not significantly different from that of the control with the pathogen only, indicating that the protective effect is due to the presence of high counts of viable predators within the carriers.

### Example 9

### Predation Dynamics of Encapsulated B. bacteriovorus in κ-Carrageenan Carriers on Biofilms

Biofilm assay was used to assess predation dynamics of encapsulated B. bacteriovorus on Pseudomonas fluorescens and E-coli ZK2686 biofilms. κ-carrageenan carriers (dissolved or ground, with and without trehalose) were added to developed biofilms of the two bacteria and quantification of biofilm biomass was assessed after 72 h incubation with the carriers using crystal violet (CV) staining measured at OD₆₀₀.

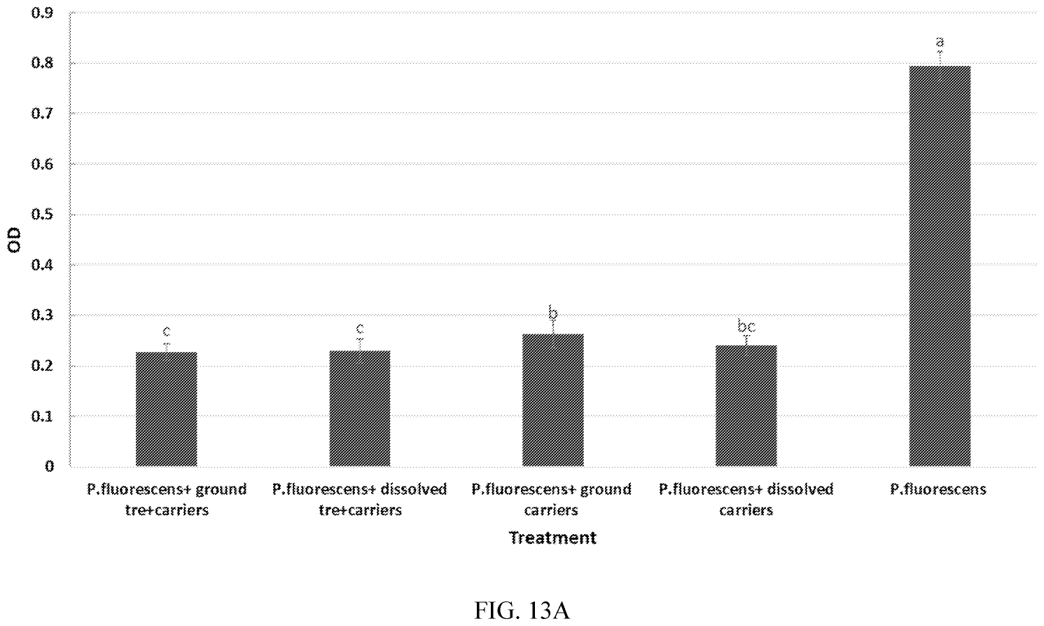

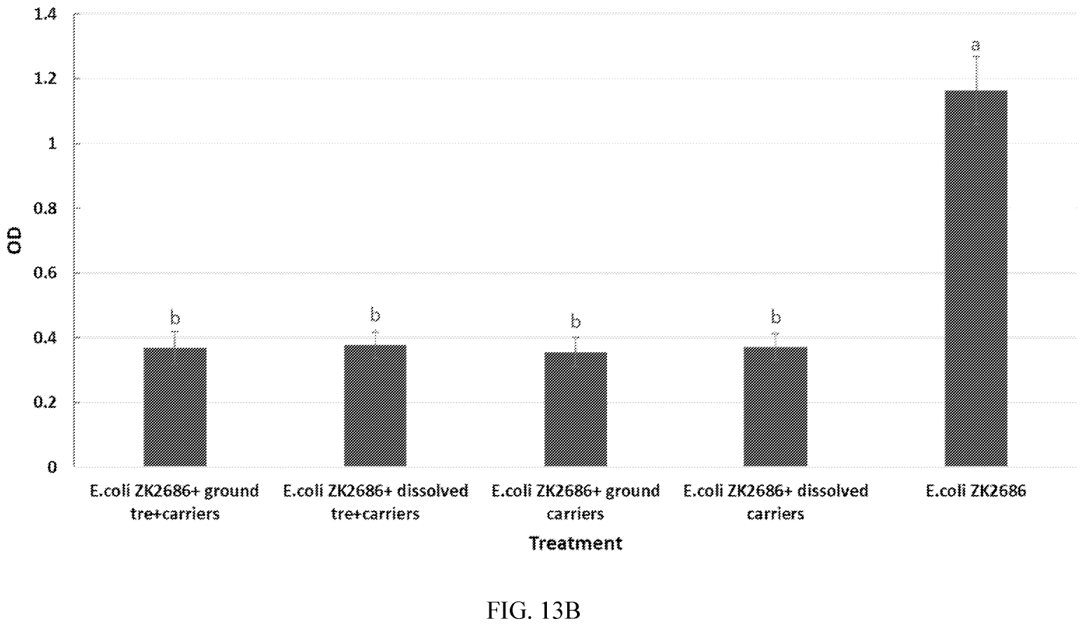

As shown in FIG. 13A, for the Pseudomonas fluorescens biofilm, a ˜65-75% reduction in crystal violet (CV) staining was observed after 72 h exposure period to the κ-carrageenan carriers. While no difference in CV staining was observed between dissolved or ground carriers, carriers containing trehalose performed better than carriers that did not contain additives.

As shown in FIG. 13B, for the E-coli ZK2686 biofilm, a ˜70% reduction in CV staining was observed after 72 h exposure period to the κ-carrageenan carriers. Furthermore, no difference was observed in CV staining between dissolved or ground carriers, nor between carriers which contained or did not contain trehalose.

### Example 10

### Preparation and Characterization of Low Methoxyl Pectin (LMP) Dried Gel Carriers

### Purification of LMP Materials

Three commercial LMP types (106 S-YA, 104 AS and 5 CS) (CPKelco, Copenhagen, Denmark) with degrees of esterification (DE) and amidation (DA) ranging from 7 to 27% and 0 to 24%, respectively, were used to prepare the carriers (Table 4). Notably, 5 CS has the lowest DE and DA. All the LMPs tested are used as food additives under EU designation E440. Since these LMPs were routinely standardized with sucrose by the manufacturer, they were purified in dialysis tubes with a 12,000-14,000 Da molecular weight cutoff (Medicell Membranes Ltd, London, England) against double distilled water (DDW) for 5 days at 4° C., changing the water three times a day. The material remaining within the dialysis tubing was freeze-dried.

TABLE 4
Chemical characteristics of the LMPs used in this study

| | | Degree of | Degree of | |
|---|---|---|---|---|
| | | esterification | amidation | pH of 2% |
| Sample details | Company | (DE) | (DA) | solution |
| Pectin LM 106 | CPKelco | 23% | 24% | 3.91 |
| AS-YA | | | | |
| Pectin LM 104 | CPKelco | 27% | 20% | 4.03 |
| AS | | | | |
| Pectin LM 5 CS | CPKelco | 7% | 0% | 4.56 |

pH-Adjustment and Pasteurization of LMP Solutions and Preparation of LMP Gel Carriers

Dialysis-purified LMP was oven-heated at 90° C. for 15 min, then dissolved in autoclaved HEPES to obtain 3% w/w LMP solution. The acidic pH was raised to 7.2 using NaOH 3M. Alternatively, the dialysis-purified LMP was dissolved in distilled water (DW) to obtain 3% w/w LMP solution. The solutions were then pasteurized by heating at 100° C. for 30 sec on a hot plate and immediately cooled on ice. Pasteurization was selected over sterilization at 121° C., since at a lower temperature, the degree of polymer hydrolysis is reduced. Gel carriers were obtained by dripping the LMP solutions into a sterile 1% (w/w) CaCl₂) solution for 20 min, with stirring. This procedure resulted in spontaneous crosslinking, producing spherical carriers with a diameter of ˜4 mm.

To evaluate the deactivation of microorganisms in the samples, carriers prepared from LMP dissolved in HEPES, as described above, were disintegrated in 2% (w/w) sodium hexametaphosphate (SHMP) solution, as detailed below, and the solutions were then plated on LB agar or potato dextrose agar (PDA) for total bacteria or yeast and molds, respectively. No colonies were detected.

### Soft Rot Enterobacteria (SRE) Growth on LMPs

To test the growth of SRE on LMPs, M9 minimal media (400 μM MgSO4, 7.5 mM (NH4)2SO4, 20 mM K2HPO4, 15 mM KH2PO4 and 0.2% (w/v) sucrose) was supplemented with 0.1% (v/v) of each of the liquid LMPs (106 S-YA, 104 AS or 5 CS), or alternatively one carrier per well. Bacterial cultures were grown overnight in LB, washed in M9, and normalized to 1×10⁷ CFU/ml. 20 μL of the normalized M9 treated-bacterial culture were transferred to fresh 180 μL of M9 minimal media supplemented with LMPs, in 96-well microtiter plates to a final bacterial concentration of 1×10⁶ CFU/ml. Bacterial growth was recorded for 48 h at 28° C. Each data point represents 8 replicates±SD, the experiment was repeated 3 times with similar results.

### LMP-Induced Plant Cell Wall Degrading Enzymes (PCWDE) Secretion by SREs

SRE cells were grown overnight at 28° C. with shaking at 150 rpm, washed twice in M9 medium and diluted to 10⁶ CFU/ml in 4 ml M9 supplemented with liquid 0.1% LMP (106 S-YA, 104 AS or 5 CS). The cultures were incubated for 24 h at 28° C. with shaking at 150 rpm. Two ml of the suspension were centrifuged at 13,000 rpm for 5 min, and 20 μL of the supernatant were used for a semi-quantitative plate assay of pectate lyase (Pel), Polygalacturonase (Peh) and protease (Prt) activity as described by Chatterjee et al. (Chatterjee et al., 1995. Appl Environ Microbiol, 61, 1959-1967). Each plate contained a specific activity medium: for Pel, 1% Polygalacturonic acid (PGA), 1% Yeast extract, 100 mM Tris-HCl (pH=8.5) (1 M stock), 0.8% Agar; for Peh, 1% Polygalacturonic acid (PGA), 1% Yeast extract, 2.2 mM ETDA (0.5 M stock), 0.8% Agar; and for Prt, 3% Gelatin, 0.4% Nutrient Broth, and 0.8% Agar. The plates were poked to form 4 mm holes, sealed with molten agarose, and the supernatants containing the enzymes were added and incubated at 28° C. for 18-24 h after which, Pel and Peh haloes were developed with 4 N HCl, and 24-36 h for Prt activity. The activity was expressed as the size of the observed haloes. Three independent experiments were carried out, each with four replicates.

### SRE Chemotaxis Towards LMP Carriers

Chemotaxis was tested using Petri dishes containing M9 minimal media, supplemented with 0.25% agar. The different LMPs (106 S-YA, 104 AS or 5 CS) were applied as spheric carriers (4 on each plate) at the center, in equal distances. Pcb was grown in fresh LB medium containing 100 μg/ml ampicillin, and incubated overnight at 28° C. with continuous shaking at 150 rpm. Next, bacterial cultures were centrifuged for 3 min at 13,000 rpm and resuspended in DDW. The bacterial strain (GFP-labeled Pcb) was applied in 5 μL drops of 1×10⁸ CFU/ml, 1.5 cm away from each carrier. After 48 h incubation at 28° C., the bacteria had migrated towards the pectin carrier, and the GFP fluorescence was detected using a Leica MZFLIII stereomicroscope (Germany) with Nikon DS-Fil digital camera.

### Optimization of 5 CS-Based LMP Carriers

Carriers were prepared as described above, except that the calcium chloride concentration was reduced to 0.35% (w/w), and the 5 CS LMP concentration was decreased to 1% (w/w). These carriers were manufactured in smaller sizes using an electronic pipette (REPETMAN®, Gilson, USA) by dripping 20 μl and 5 μl drops into the cross-linking solution, yielding carriers with diameters of ˜3- and 2-mm, respectively. Further optimization of 5 CS carriers was achieved by the addition of gelatin, prepared by dissolving gelatin powder (Bovine skin gelatin, Sigma, USA) into HEPES, followed by autoclaving at 121° C. for 20 min. The solutions were mixed to final concentrations of 1% pectin+2% gelatin or 1% pectin+5% gelatin, and carriers obtained from 5 μl drops in a 0.35% CaCl₂) solution. Carriers were then dried in a vacuum oven (Binder, Germany) at 37° C. and 400 mbar.

### Sphericity of Pectin Gel Carriers

Digital pictures of the LMP gel carriers were acquired under a binocular (model 5ZX16, Olympus America Inc., Center Valley, PA, USA) and their dimensions were measured using the image analysis software Image J (version 1.48 d, NIH, Bethesda, MD, USA). Sphericity was defined by calculating the ratio of the sphere's small to the large diameter:

$$Aspect ratio = \frac{{D}_{minimum}}{{D}_{maximum}}$$

Values closer to 1 indicate rounder beads (a value of 1 indicates a perfect sphere). Twelve replicates from two different batches were measured.

### Mechanical Properties of the Carriers

The LMP gel carriers were compressed between parallel lubricated plates to failure, at a constant deformation rate of 10 mm/min with an Instron Universal Testing Machine (UTM) (model 5544, Instron Corporation, Canton, MA, USA). Software Bluehill 3 enabled data conversion of the Instron's voltage versus time measurements into digitized force-deformation values. The stress (o) and strain (EE) were defined as:

$$σ = F / {A}_{o}$$

$${ε}_{E} = Δ H / {H}_{o}$$

where F is the force at break (N), Hₒ the carrier's initial height (m), ΔH the total deformation (m), and Aₒ the initial cross-sectional area of the carrier (m²).

### Swelling Ratios Measurements of LMP Gel Carriers

LMP gel carriers were introduced into plastic tubes containing DW. To avoid volume-dilution effects, the liquid-to-specimen volume ratio was in excess of 100:1. Periodically, carriers were removed from the tubes, wiped with filter paper, and weighed (±0.001 g) on an analytical balance (Precisa, Dietikon, Switzerland). The test was ended when the specimens reached constant weight (equilibrium, within 4-5 days).

The equilibrium swelling ratio by weight was calculated as follows:

$$Swelling ratio = \frac{W ( final )}{W ( initial )}$$

where W (initial) is the initial specimen weight (g) and W (final) is the final specimen weight at equilibrium (g).

### Reswelling Capacity of Dried LMP Carriers

Dried carriers introduced to DW at 25° C. were periodically weighed (+0.001 g) on an analytical balance (Precisa, Dietikon, Switzerland). The test was ended when the specimens reached a constant weight (equilibrium) usually after less than ˜24 h. Noticeably, carrier swelling started within a few minutes.

Reswelling capacity was calculated as:

$$Reswelling ( \% ) = \frac{W ( final )}{W ( initial wet state )} \times 100$$

where W (initial wet state) is the specimen weight as gel carrier before drying (g), and W (final) is the reswelled dried specimen weight at equilibrium (g).

### Preparation of LMP Carriers for the Encapsulation of BALOs

The 5 CS LMP carrier composition was prepared as above at a final concentration of 1% LMP+5% gelatin. Concentrated bdelloplasts were then added at a 1:9 volumetric ratio to the LMP-gelatin solution, and carriers obtained from 20 μl or 5 μl drops in a 0.35% CaCl₂) solution, followed by vacuum drying, as above.

### Encapsulation Efficiency

In order to release the encapsulated bdelloplasts from the LMP dried carriers, carriers were mixed with a sterile 2% sodium hexametaphosphate (SHMP) solution and shaken until complete dissolution. A previous study and preliminary experiments showed no harmful effect of SHMP on the predator (Sivakala et al., 2021. Journal of Applied Microbiology, 131, 2971-2980). The density of viable cells in the suspensions was obtained by dilution plating and PFU counts in double layered agar. EE was calculated according to the description in Example 2 herein above.

### Scanning Electron Microscopy (SEM)

Dried LMP gel carriers with encapsulated predators and the bacteria that reside within the carriers were visualized using SEM and high-resolution SEM (Tokyo, Japan), respectively, as previously described in Example 2 herein above.

### SRE-Induced Carrier Degradation

Pcb was grown as above, subcultured in fresh LB to an OD₆₀₀ of ˜0.2 and washed twice in aHEPES buffer. OD₆₀₀ was adjusted to 0.2, then diluted to yield ˜10⁷ CFU/ml. Pcb-induced degradation of LMP gel carriers prepared from the different LMPs, and of different preparations of 5 CS-based carriers was measured in double diluted NB medium (DDNB) amended with 2 mM CaCl₂) and 3 mM MgCl₂·6H₂O, in shaking flasks at 28° C. One carrier was placed in 10 ml of DDNB with or without Pcb, in 6 replicates from two different batches. The carriers were removed after 24, 48 and 72 h from the tubes, wiped with sterile filter paper and weighed (±0.001 g) under sterile conditions on an analytical balance (Precisa, Dietikon, Switzerland). Weight change was calculated as the percentage difference between the carriers' initial and final weights.

### Predation Dynamics of SRE-Released Encapsulated Predators

Predation by encapsulated bdelloplasts of B. bacteriovorus HD100-Td-tomato in LMP dried carriers (obtained from 5 μl drops) was tracked by fluorescence (predator growth) and OD (prey decay) in a plate reader incubated at 28° C., with shaking and measurement performed every 20 min (Tecan Spark 10M, Switzerland). The fluorescence detectable threshold for the lower and upper levels corresponded to ˜4×10⁷ and ˜10⁹ PFU/ml B. bacteriovorus HD100, respectively. One ml of Pcb suspension (OD₆₀₀=10) was added to 9 ml amended DDNB and 200 μl aliquots pipetted onto wells of clear black flat-bottom 96-well plates (Greiner-Bio One, Germany) containing 1, 2 or 3 carriers in a well. The experiments were performed twice independently and included three technical replicates per treatment.

### Example 11

### Physical and Mechanical Properties of LMP Carriers

Adjustment of pH and pasteurization significantly and differentially affected the carriers, depending on the LMP material. Table 5 demonstrates that while the diameter of 106 AS-YA and 5 CS carriers decreased, that of 104 AS was not significantly changed. However, an increase in carriers' sphericity was consistently detected. Table 6 presents results for load at break, stress and strain at break measurements, showing increased values in all cases, but with large differences between the formulations tested. Table 7 presents the carriers' swelling ratio of the carriers following pH adjustment and pasteurization. The stability of the carriers was investigated by immersing them in DW; The carriers did not disintegrate but rather swelled or contracted by up to ˜20%. (Table 7). Overall, the enhanced features of the pH-adjusted, pasteurized carriers may contribute to their durability during application.

TABLE 5
Physical properties of LMP gel carriers without (−)
and with (+) pH adjustment and pasteurization.

| | | Carrier | Carrier |
|---|---|---|---|
| | Carrier | initial | sphericity |
| | diameter | weight | Aspect ratio |
| Carrier type | (mm) | (mg) | (—) |
| LM 106 AS-YA (−) | 4.37^{a} ± 0.19 | 41.2^{b} ± 2.0 | 0.92^{bc} ± 0.01 |
| LM 106 AS-YA (+) | 4.21^{b} ± 0.25 | 42.0^{b} ± 2.6 | 0.94^{ab} ± 0.01 |
| LM 104 AS (−) | 4.20^{b} ± 0.33 | 41.0^{b} ± 3.2 | 0.92^{bc} ± 0.02 |
| LM 104 AS (+) | 4.32^{ab} ± 0.15 | 43.7^{b} ± 1.8 | 0.95^{a} ± 0.01 |
| LM 5 CS (−) | 4.17^{b} ± 0.25 | 46.9^{a} ± 4.0 | 0.91^{c} ± 0.03 |
| LM 5 CS (+) | 3.98^{c} ± 0.28 | 46.9^{a} ± 4.0 | 0.95^{a} ± 0.01 |

Different superscript letters in a column indicate significant differences (Student's t-test, P < 0.05). Results are averages of at least 10 replicates ± SD from two different batches.

TABLE 6
Mechanical properties of LMP gel carriers without (−) and
with (+) pH adjustment and pasteurization.

| | Load at | | Strain at |
|---|---|---|---|
| Carrier type | break(N) | Stress (MPa) | break (%) |
| LM 106 AS-YA (−) | 13.05^{c} ± 1.94 | 0.87^{c} ± 0.15 | 69.5^{b} ± 4.8 |
| LM 106 AS-YA (+) | 20.31^{a} ± 1.59 | 1.47^{a} ± 0.23 | 75.0^{a} ± 5.4 |
| LM 104 AS (−) | 9.93^{d} ± 0.93 | 0.72^{d} ± 0.11 | 68.5^{b} ± 5.5 |
| LM 104 AS (+) | 14.56^{b} ± 1.65 | 0.99^{b} ± 0.11 | 74.3^{a} ± 3.8 |
| LM 5 CS (−) | 2.13^{f} ± 0.14 | 0.15^{f} ± 0.01 | 42.0^{d} ± 4.8 |
| LM 5 CS (+) | 5.89^{e} ± 0.37 | 0.48^{e} ± 0.09 | 51.8^{c} ± 4.5 |

Different superscript letters in a column indicate significant differences (Student's t-test, P < 0.05). Results are averages of at least 10 replicates ± SD from two different batches.

TABLE 7
Equilibrium swelling ratio values of pectin gel carriers without (−) and
with (+) pH adjustment and pasteurization in DW at 25° C.

| | Carrier swelling |
|---|---|
| Carrier type | ratio (—) |
| LM 106 AS-YA (−) | 1.21^{a} ± 0.01 |
| LM 106 AS-YA (−) | 1.17^{b} ± 0.01 |
| LM 104 AS (−) | 1.11^{c} ± 0.04 |
| LM 104 AS (+) | 1.22^{a} ± 0.01 |
| LM 5 CS (−) | 0.91^{e} ± 0.01 |
| LM 5 CS (+) | 0.96^{d} ± 0.01 |

Different superscript letters in a column indicate significant differences (Student's t-test, P < 0.05). Results are averages of at least 10 replicates ± SD from two different batches.

### Example 12

### Soft Rot Enterobacteria (SRE) Growth on LMPs

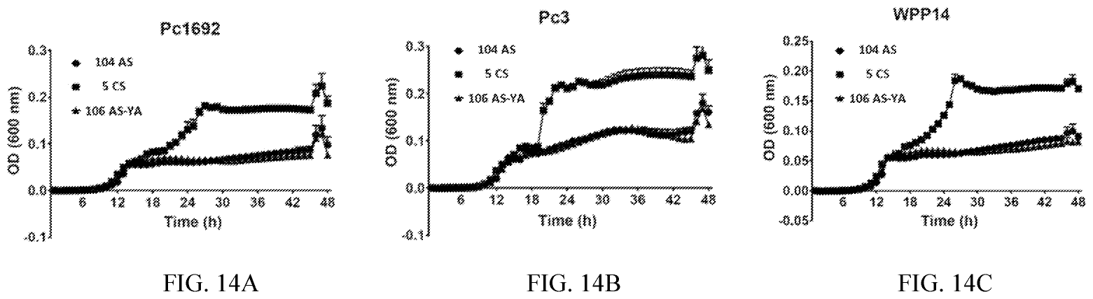

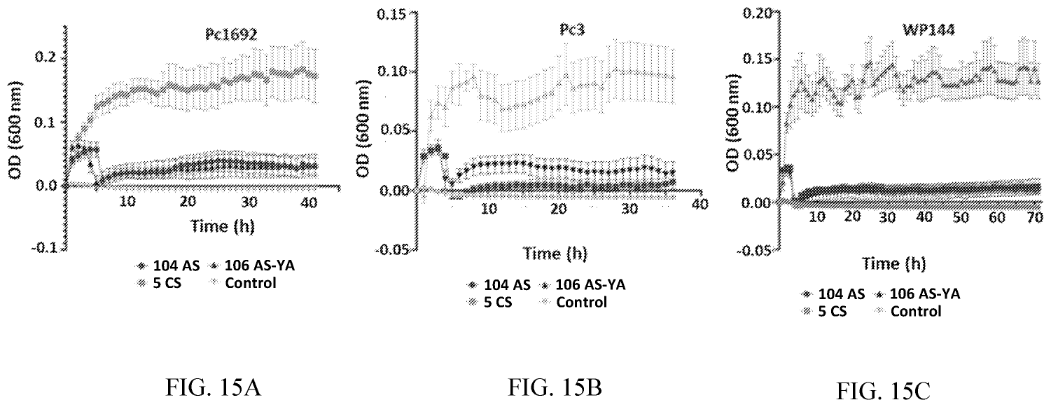

SRE growth on LMPs 106 S-YA, 104 AS and 5 CS in solution or as gel carriers was tested in M9 minimal media, with the materials acting as the sole carbon source. Initially, calibration was performed to determine inoculum size and LMP concentration. Consequently, 0.1% (w/w) LMP (or alternatively, 1 carrier per well in 96-well plates) and an inoculum of 10⁶ CFU/ml were selected. Solutions of all three materials enabled SRE growth, with 5 CS yielding significantly higher populations (FIG. 14). Very poor growth was achieved with the 106 S-YA, 104 AS carriers in contrast to the 5CS carriers which yielded populations at levels similar to those obtained in 5CS solutions, suggesting that the 5 CS carriers were solubilized and consumed (FIG. 15).

### Example 13

### LMP-Induced Plant Cell Wall Degrading Enzymes (PCWDE) Secretion by SREs

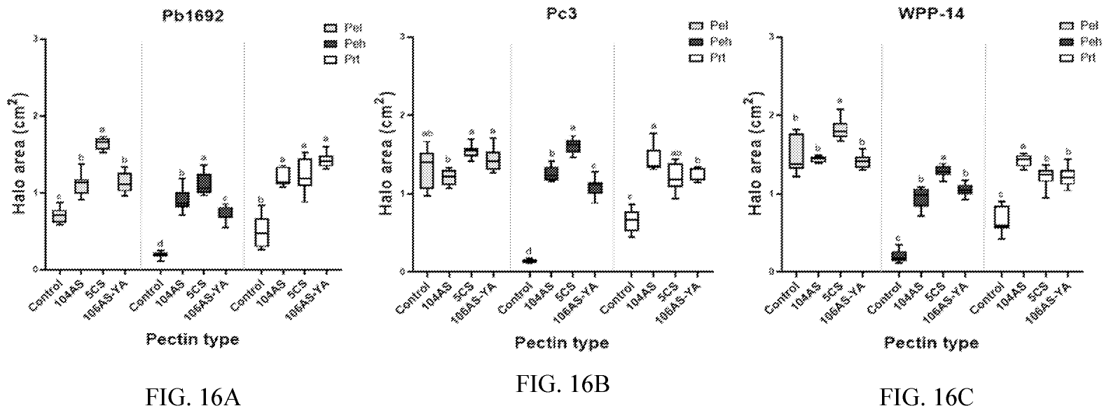

LMP-dependent PCWDE secretion was tested by growing the soft rot Enterobacteria (SREs) with LMPs as a sole carbon source for 8 hours, using the culture supernatants in Petri dishes containing a medium specific for each of the pectinolytic enzymes pectate lyase (Pel) and polygalacturonase (Peh), and for the protease (Prt). All LMP solutions induced the secretion of PCWDEs. Among them, 5CS induced significantly higher Pel and Peh activities in the SRE strains tested. However, the different LMP solutions did not induce differential Prt secretion (FIGS. 16A-16C). These results are consistent with the growth dynamics measurements shown in FIGS. 14A-14C and FIGS. 15A-15C and indicate that 5 CS pectin is highly suited as a material inducing SRE pectinolytic activities for the release of BALOs in pectin carriers.

### Example 14

### SRE Chemotaxis Towards LMP Carriers

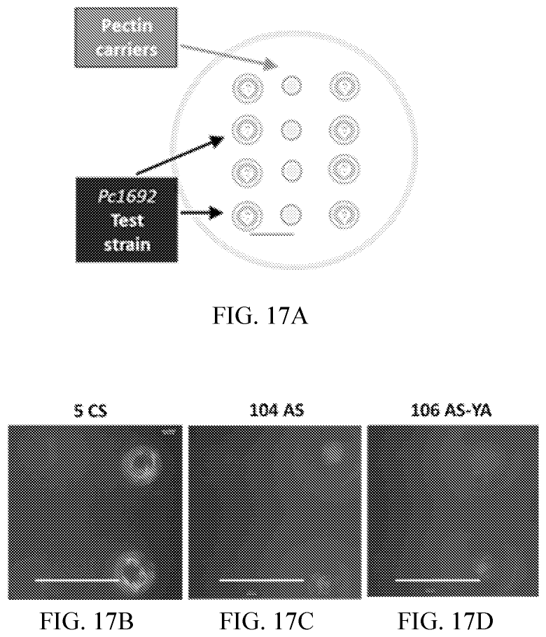

Chemotaxis of a green fluorescent protein (GFP)-labeled Pcb strain towards the different LMP sources was tested in soft minimal agar plates (FIG. 17A). FIGS. 17B-17D show that after 48 hours, the bacteria had migrated and populated the shell of the 5 CS LMP carrier (FIG. 17B), possibly also multiplying within it, while much lower fluorescence emanated from the 104 AS and 106 AS-YA carriers (FIGS. 17C and 17D, respectively), suggesting lower associated bacterial populations. Based on these results, it can be concluded that Pcb is attracted to the LMP 5 CS carrier within which the cells multiply.

### Example 15

### Degradation Dynamics of LMP Carriers by SREs

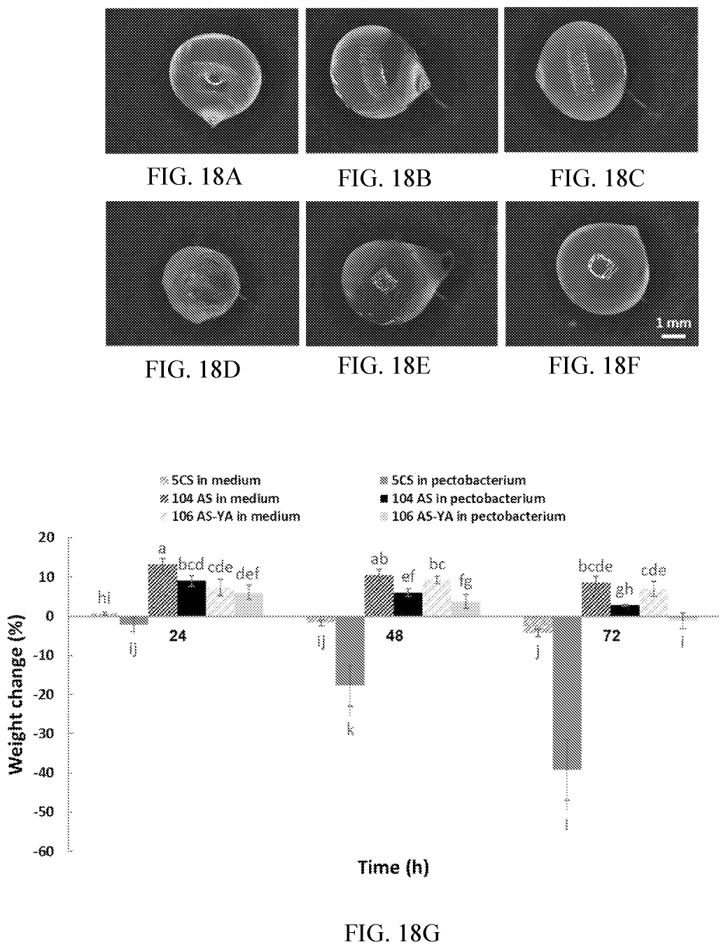

The degradation of the different LMP gel carriers by SREs was tracked in DDNB with or without Pcb, for 3 days. At the end of this period, the size of the 5 CS LMP carrier in the presence of Pcb was smaller, and the surface rougher (FIG. 18D), as compared to the same carrier immersed in medium only (FIG. 18A). In contrast, there were no noticeable changes in the appearance of the two other carriers (FIGS. 18E and 18F). FIG. 18G shows that all the carriers underwent weight changes in the medium alone, probably due to gradual swelling for 104 AS and 106 AS-YA or shrinking for 5 CS, until equilibrium was reached. The tendency of each type of carrier to swell or shrink in DDNB is consistent with the trends observed when those carriers were immersed in DW (Table 8). FIG. 18G further shows that all LMP carriers lost more weight when incubated with Pcb as compared to medium alone, wherein the differences increased over time. The 5 CS carrier significantly lost more weight, decreasing by about 40% of its initial weight within 72 hours of incubation with Pcb.

It was further observed in Table 8 that in the DDNB control treatment, the strength of the different carriers was reduced as compared to before treatment (Table 6). These results could be explained by the diffusion of cross-linking ions from the carriers to the medium, weakening the structure of the carriers. Incubation with Pcb significantly weakened and fragilized all the carriers (Table 8).

TABLE 8
Mechanical properties of LMP carriers incubated
for 72 h in DDNB without or with Pcb

| | Load at | | Strain at |
|---|---|---|---|
| Carrier type | break (N) | Stress (MPa) | break (%) |
| LM 106 AS-YA (DDNB) | 8.07^{a} ± 0.90 | 0.60^{a} ± 0.04 | 74.5^{a} ± 4.3 |
| LM 106 AS-YA (Pcb) | 1.57^{d} ± 0.52 | 0.11^{d} ± 0.04 | 58.8^{bc} ± 5.6 |
| LM 104 AS (DDNB) | 6.13^{b} ± 0.86 | 0.47^{b} ± 0.07 | 74.2^{a} ± 5.0 |
| LM 104 AS (Pcb) | 1.27^{d} ± 0.19 | 0.10^{d} ± 0.02 | 61.5^{b} ± 4.0 |
| LM 5 CS (DDNB) | 4.58^{c} ± 0.54 | 0.35^{c} ± 0.04 | 55.3^{c} ± 5.6 |
| LM 5 CS (Pcb) | 1.08^{d} ± 0.11 | 0.11^{d} ± 0.01 | 45.5^{d} ± 6.5 |

Different superscript letters in a column indicate significant differences (Student's t-test, P < 0.05). Results are averages of 6 replicates ± SD, from two different batches.

### Example 16

### Optimization of 5CS-Based Pectin Carriers

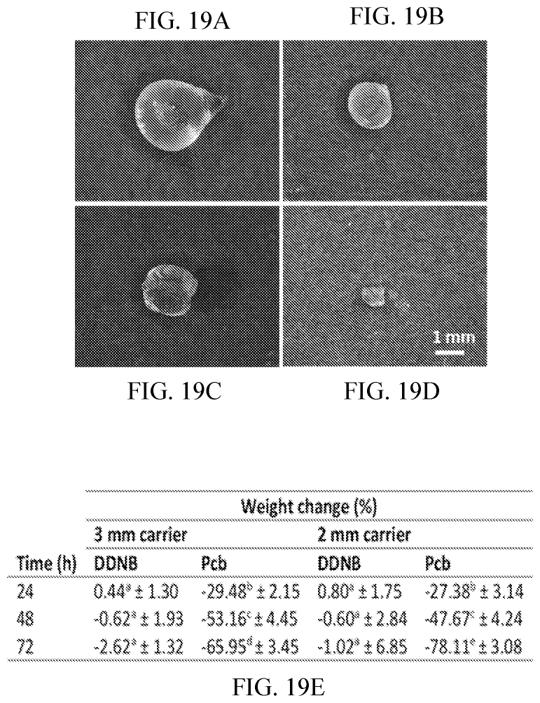

5 CS LMP carriers were optimized to enable faster SRE-induced degradation, examining the effect of reducing CaCl₂) cross-linker concentration and carrier size from 1% to 0.35% and from 4 mm to diameters of 2 or 3 mm, respectively. FIGS. 19A-19E show that all the carriers exposed to Pcb in DDNB lost significantly more weight than the DDNB controls. Furthermore, during the first 48 h of incubation with Pcb, the differently sized carriers lost 50% of their weight. However, after 72 hours, the smaller carrier decreased by about 80% of its initial weight as compared to 65% for the larger carrier (FIG. 19E). This value is twice higher than the one obtained with the previous formulation (FIGS. 18A-18G).

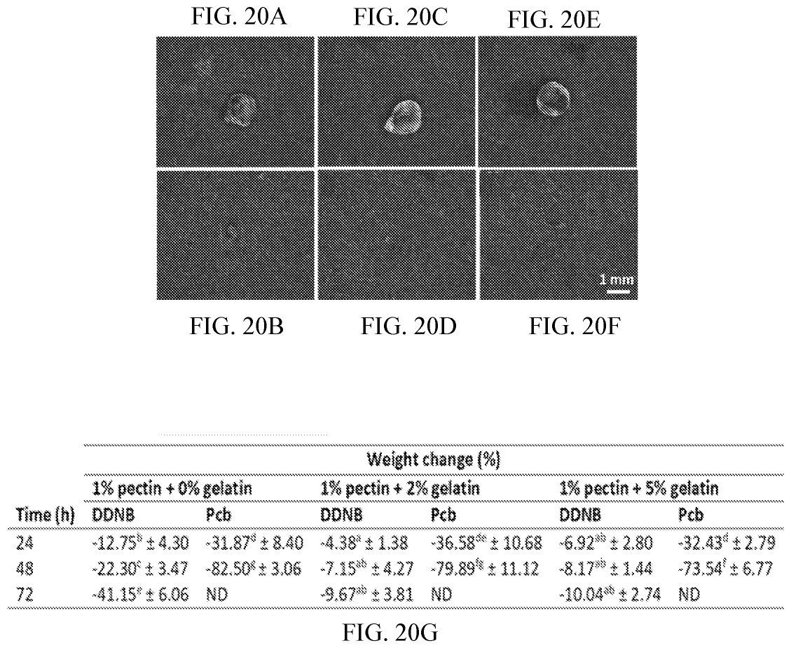

Reducing LMP concentration from 3 to 1% resulted in carriers becoming less stable, as revealed by the large weight loss occurring even in the absence of Pcb (FIG. 20G). Since gelatin can form ionic interactions with LMP and by that improve gel performance, the effect of 2% and 5% gelatin addition to the matrix was examined. FIGS. 20A-20G demonstrate that gelatin addition stabilized the carriers, as they lost significantly less weight in the absence of Pcb. However, in the presence of Pcb, the carriers with the different gelatin concentrations shrank by about 80% of their initial weight within 48 h. By 72 h, their weight was no longer measurable by the existing means (FIG. 20G).

The effect of vacuum drying of the LMP carriers was further examined. The reswelling capacity of dried 5 CS LMP carriers is an important parameter because enzymatic decomposition may be more effective in carriers exhibiting good reswelling, indicating a lower structural density. Table 9 demonstrates that the addition of gelatin to the LMP carriers resulted in improved reswelling capacity.

TABLE 9
Reswelling capacity of dried carriers comprising
5 CS LMP and gelatin in DW at 25

| LMP in sample (%) | Gelatin in sample (%) | Reswelling (%) |
|---|---|---|
| 1 | 0 | 4.6^{a} ± 0.1 |
| 1 | 2 | 19.0^{b} ± 0.2 |
| 1 | 5 | 46.6^{c} ± 0.3 |

Different superscript letters in a column indicate significant differences (Student's t-test, P < 0.05). Results are averages of 10 replicates ± SD from two different batches.

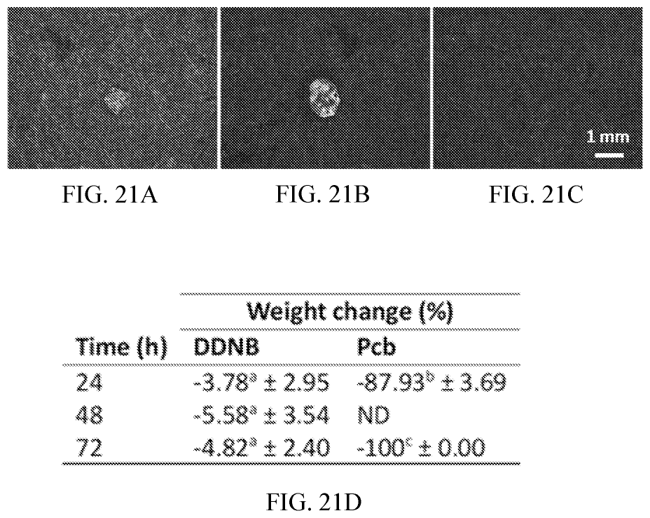

Accordingly, dry carriers containing 1% LMP+5% gelatin were first rewetted for 24 h in DW, then incubated for 24 h with Pcb in DDNB, leading to a weight loss of about 88% (FIG. 21D), a value only attained within 48 h without the drying and rewetting steps as can be seen in FIG. 20G. After 48 h, weights were no longer measurable and after 72 h, the carriers completely disintegrated (FIGS. 21A-21D).

Finally, the ability of the optimized carriers to encapsulate and preserve the viability of a mixture of B. bacteriovorus AP cells and bdelloplasts was examined. A high number of viable predatory bacteria (1.4×10⁹ PFU/g carriers) was detected within the LMP dried carriers, although a 2-log reduction followed the dehydration process. The initial entrapment of the predators in the carriers prior to drying was about 57% (Table 10).

TABLE 10
Density and entrapment efficiency of B. bacteriovorus
in optimized, dried pectin-gelatin carriers.

| | |
|---|---|
| Entrapment efficiency (%) | 57.2 ± 3.6 |
| Predator count after vacuum- | (1.4 ± 0.2) × 10⁹ |
| drying (PFU/g carriers) | |
| Survival (%) | 0.93 ± 0.06 |

Results represent the average ± SD of two independent experiments with three technical replicates for each treatment. In each test, five carriers were evaluated.

### Example 17

### Scanning Electron Microscopy of LMP and Gelatin Carriers

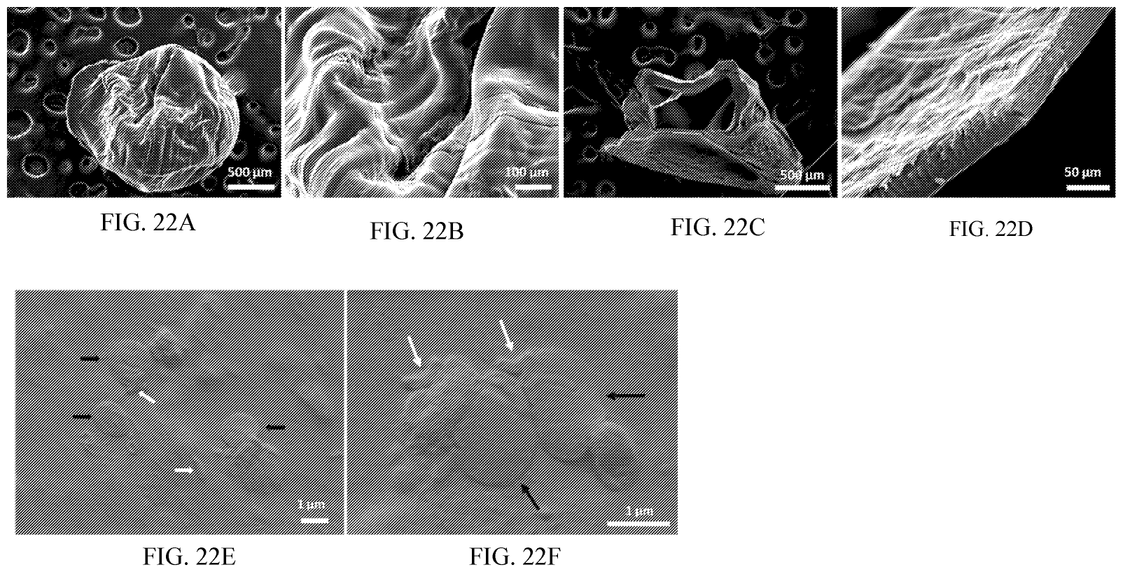

Observation under SEM revealed a thin shell wall and a hollow core, which may explain the rapid degradation of the optimized carriers by Pcb. FIGS. 22A-22D reveal a relatively smooth surface with few salt crystals. Further analysis with high-resolution SEM enabled the visualization of B. bacteriovorus, both as AP cells and as elongating cells within bdelloplasts, embedded within the matrix (FIGS. 22E-22F). AP cells could be seen with their flagellum, suggesting that upon release, the predator would readily be active. This mixed population results from the protocol used to obtain bdelloplasts, in which AP cells are twice the concentration of prey.

### Example 18

### Predation Dynamics of SRE-Released Encapsulated Predators

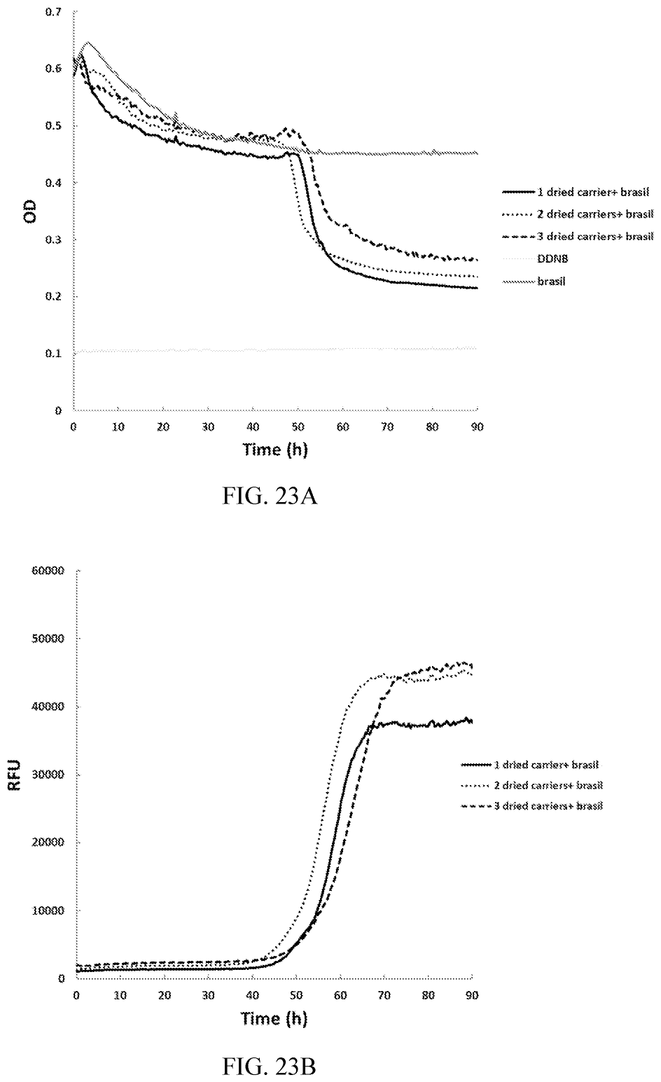

To test the predation efficiency of the predator encapsulated in optimized dried LMP carriers, B. bacteriovorus HD100 expressing the Td-tomato fluorescent protein was immobilized in the carriers and vacuum dried. Pcb was inoculated in microwells along with 1, 2 or 3 carriers (FIGS. 23A-23B). All treatments with carriers resulted in a significant decrease in the pathogen population size, as reflected by the decrease in OD values (FIG. 23A), and in an increase in the predator population, as shown by fluorescence (FIG. 23B). The final fluorescence level increased with the number of carriers, but final prey OD values were not concurrent (i.e., lower OD values were obtained with a lower number of carriers). It should be noted that in this range, RFUs are linear, and thus reflect a circa 1.5-fold difference in predator population density. Without wishing to be bound by any theory or mechanism of action, it is contemplated that the controlled release of the predators is operated by the gradual degradation of the carrier's matrix by the enzymatic activity of the pathogen, resulting in predation upon Pcb. Supporting this, no plaques were observed from samples collected after 96 h from the medium in control wells containing carriers without Pcb. This indicates that the carriers are not leaky, and predator release requires the degradation of the matrix.

### Example 19

### Protection of Potato Tubers Against Soft Rot by Bdellovibrio Encapsulated Formulations

### Bacterial Strains, Media, and Growth Conditions

The predatory bacteria used in this study was Bdellovibrio bacteriovorus HD100 (ATCC 15356). Escherichia coli ML35 (ATCC 43827) was used as prey bacteria.

E. coli was grown in LB medium at 37° C. in shaking flasks at 250 rpm, from single colonies originating from laboratory stocks kept at −80° C. Overnight cultures were centrifuged at 3620 g for 10 min at 4° C., and the cell pellet was washed and resuspended in HEPES buffer pH 7.8 amended with 2 mM CaCl₂) and 3 mM MgCl₂·6H2O (amendedHEPES—aHEPES), to a final optical density (OD₆₀₀) of 10. The E. coli suspension was further concentrated to yield ˜10¹¹ colony forming units (CFU)/ml. The stock suspensions were stored at 4° C. until use, for up to two weeks.

To initiate predatory co-cultures, a bacteriological loopful from the frozen stock of the predator was transferred to a flask containing 1.5 ml of an OD₆₀₀=10 E. coli suspension diluted in 15 mL of aHEPES. The co-cultures were incubated at 28° C. and 250 rpm until the culture's turbidity reached below OD₆₀₀=0.1. Then the suspension was filtered through a 0.45 μm filter (Sartorius). Thereafter, 5 ml of the filtered predatory cells from the starter culture were inoculated into 40 ml of aHEPES containing 5 ml of an OD₆₀₀=10 E. coli suspension in a 250 mL flask. The flask was shaken at 250 rpm at 28° C. Growth of the predator was monitored by a decrease in turbidity of the suspension. After most of the prey cells were lysed, the suspension was filtered through a 0.45 μm filter (Sartorius) yielding ˜10⁹ plaque forming units (PFU)/ml of predatory cells. The filtrate was stored at 4° C. until use for up to one day, without viability loss. To further concentrate the filtered predatory cells, the suspension (50 ml) was centrifugated at 12,497 g for 20 min at 10° C. and the cell pellet was resuspended in 0.5 ml of aHEPES to a final concentration of ˜10¹¹ PFU/ml of predatory cells. To create large amounts of bdelloplasts for encapsulation, synchronized cultures of the predator were obtained by adding 250 μl of the concentrated E. coli suspension to 500 μl of the concentrated AP cell suspension (1:2 prey: predator) incubated for about 1 hour at 28° C. and 250 rpm. This protocol resulted in an approximate ratio of 1:1 (AP cells: bdelloplasts). Bdelloplast formation was validated by phase microscopy. Prey and predator cultures were routinely counted by dilution plating as CFU per ml and PFU per ml, respectively. The latter plating was performed in double-layered agar (Jurkevitch 2012).

For the infection assays of potato tubers, Pectobacterium brasiliense (Pcb 1692) was used. Bacteria cells were grown overnight in LB at 28° C., 150 rpm, washed twice in M9 medium and normalized to 5×10⁶ CFU/ml or 5×10⁸ CFU/ml.

### Preparation of κ-Carrageenan-Based Carriers for Predator Encapsulation

κ-carrageenan dispersion (1.67% (w/w)) was prepared by adding k-carrageenan powder (Sigma, USA) to DW. Trehalose (Sigma, USA) was then added to a final concentration (w/w) of 5%. The mixture was then brought to boil to complete gum dissolution. Concentrated bdelloplasts were then added at a 1:9 volumetric ratio to the κ-carrageenan-based solution at 40° C. (yielding a final concentration of 1.5% carrageenan). This final mixture was dripped into a 0.5% (w/w) sterile solution of KCl (Biolab Chemicals, Israel) at volumetric ratio of 1:100 and stirred for 20 min. A spontaneous cross-linking reaction produced spherical gel/wet carriers. The gel carriers were then dried in a vacuum oven (Binder, Germany) at 37° C. and 400 mbar. To obtain powder, the dry carriers were milled by electric grinder (Graetz, Germany).

### Preparation of Pectin-Based Carriers for Predator Encapsulation

5 CS pectin (CPKelco, Copenhagen, Denmark) with degree of esterification (DE) of 7% and amidation (DA) of 0% was used to prepare the carriers. Before use, the pectin was purified and pasteurized. Gelatin solution was prepared by dissolving gelatin powder (Bovine skin gelatin, Sigma, USA) into HEPES (or HEPES with trehalose), followed by autoclaving at 121° C. for 20 min. The solutions were mixed to final concentrations of 1% pectin+5% gelatin or 1% pectin+5% gelatin+5% trehalose. Concentrated bdelloplasts were then added at a 1:9 volumetric ratio to the solution, and then the suspension was dripped into a sterile 0.35% CaCl₂) solution, followed by vacuum drying at 37° C. and 400 mbar. To obtain powder, the dry carriers were milled by electric grinder (Graetz, Germany).

### Bacterial Enumeration in the Carriers

For quantitative measurements of cell viability, dried carrageenan-based or pectin-based carriers were immersed in sterile DW or sterile 2% sodium hexametaphosphate (SHMP), respectively. The mixtures were then shaken until the carriers were totally dissolved. PFUs, representing viable cells, were counted by dilution plating in double layered agar.

### Stability of Encapsulated Predators Under Storage

The viability of encapsulated predators in carrageenan-based or pectin-based carriers, kept in hermetically sealed glass flasks containing silica gel, was measured during a one year-long period or until no viable bacteria were detected, stored at 3 different temperatures: 25° C., 4° C. and −20° C. Liquid suspensions of AP cells, prepared as described herein above, stored at the same temperature, served as a control. Bacterial enumeration in the carriers/suspensions was performed as detailed above, at intervals spanning from weeks to months.

### Infection Assays of Potato Tubers and Carrier-Based Protection in Small Vessels

White-skinned potato cultivar ‘Nicola’ (with average weight of ˜30 grams) was sourced from local supermarkets. Potatoes were surface sterilized by dipping for 20 min in a 0.5% sodium hypochlorite (NaOCl) solution, washed twice with sterile DW for 15 min, and left to dry in a laminar flow sterile hood. P. brasiliense suspension was prepared as described above. Potato tubers were inoculated by dipping them for 10 min in the pathogen culture at a concentration of 5×10⁸ CFU/ml. Following inoculation, the tubers were planted each in a 6 cm² cell of a sterilized plant pot, containing autoclaved all-purpose potting garden mix soil (ECOTERRA+, rich in nutrients composed of coconut coir, peat, and compost 70:20:10). The treatments (2 ml of BALOs suspensions) were applied on top of each potato tuber before covering with soil, or applied to the soil under the tuber, or near the corners of the pot. For the control treatment, 2 ml of DW were used. Test treatments included dissolved carrageenan-based carriers (in tap water to yield ˜10⁸ PFU/ml) or predator suspensions (˜10⁶ or ˜10⁸ PFU/ml). The pots were introduced in plastic boxes in a controlled environment chamber at 25° C./28° C. (±1° C.), high relative humidity, and 16 h of fluorescent light-8 h dark photoperiod. Moisture level was maintained by covering the plastic boxes/the pots with plastic wrap. Seven or 14-days post infection, the tubers were extracted and average decay (%) and number of infected potatoes were recorded. Disease Index was calculated as: Average decay (%)×Number of infected potatoes. All experiments were repeated four times, with 15 replicates per treatment.

### Infection Assays of Potato Tubers and Carrier-Based Protection in a Net-House

The experiments were conducted in a net-house. Potatoes (average tuber weight of ˜85 grams) were planted and irrigated twice a week by over-head spraying. The experiments were carried out for one month. Potatoes were prepared as above and treated with 2 ml of dissolved carrier suspension for a total of 4 times. The initial application was on top of the potato tuber before covering with soil, the following applications were made directly onto the soil. All experiments were repeated twice, with 32 replicates per treatment.

The methods described in this example were used in the Examples described herein below.

### Example 20

### Stability of Encapsulated Predators Under Storage

To examine the shelf life of dry formulations of encapsulated BALOs, the survival of B. bacteriovorus HD100 encapsulated in two different carriers: carrageenan-based and pectin-gelatin based carriers, was evaluated during one year storage at three temperatures: 25° C., 4° C. and −20° C., and compared to that of liquid suspensions of predator AP cells stored at the same temperatures.

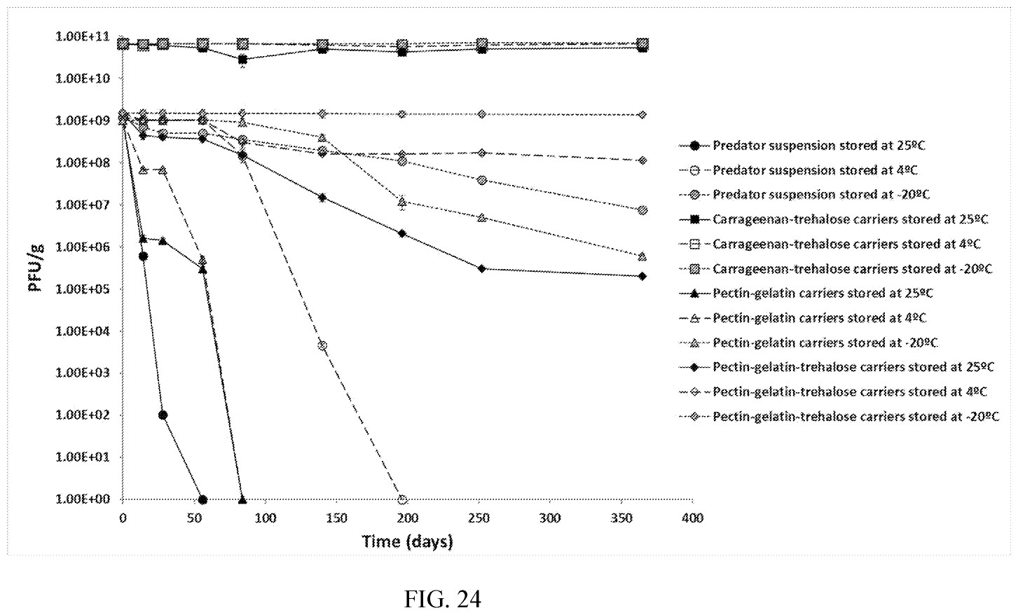

The carrageenan-trehalose carriers showed high stability at all temperatures (FIG. 24). In contrast, the cell density in the liquid suspensions at 25° C. decreased drastically: after one month only 1.0×10² live bacteria were detected in the suspension, and after two months no live bacteria were detected (Table 11 and FIG. 24). At 4° C., the decay rate of the bacteria in liquid suspensions was slower, yet survival decreased sharply after five months until no viable cell could be detected at seven months of storage. While storage at −20° C. improved survival, the concentration of predators in the suspension was still reduced by about two orders of magnitude (FIG. 24).

TABLE 11
Survival of B. bacteriovorus HD100 in carrageenan-
trehalose carrier and in liquid suspensions at 25° C.

| Time | Predator suspension | Carrageenan-trehalose carrier |
|---|---|---|
| (months) | (PFU/ml) | (PFU/g carrier) |
| 0 | 1.2 × 10⁹ | 6.5 × 10¹⁰ |
| 1 | 1.0 × 10² | 6.0 × 10¹⁰ |
| 12 | 0 | 5.5 × 10¹⁰ |

The pectin-gelatin dried carriers displayed improved survival over the bacterial suspensions at room temperature with a lower decay rate. However, after 3 months, no viable cell could be detected in the carriers at both 25° C. and 4° C. (FIG. 24). The addition of trehalose dramatically improved survival at all temperatures with almost no loss, and a decrease of about one order of magnitude in viability being recorded with storage at −20° C. and at 4° C., respectively (FIG. 24). These results demonstrate the superior stability of encapsulated Bdellovibrio formulations as compared to liquid predator suspensions, and thus their long shelf-life.

### Example 21

### Protection of Potato Tubers Against Soft Rot by Predator Formulations Under Controlled Conditions

To evaluate the protection of potato tubers against Pectobacterium brasiliense by encapsulated Bdelovibrio bacteriovorus HD100, potato tubers were dipped in P. brasiliense suspension, and then treated with water as a control or with the predators encapsulated in carrageenan-trehalose, obtained after dissolution of the dried carriers in water, which were applied on top of the potato tuber. The incubation continued for 7 days at 25° C. under humid conditions. The effect of the encapsulated predators was compared to the effect of AP suspensions at the same density (10⁸ PFU/ml) which were also applied on top of the potato tubers.

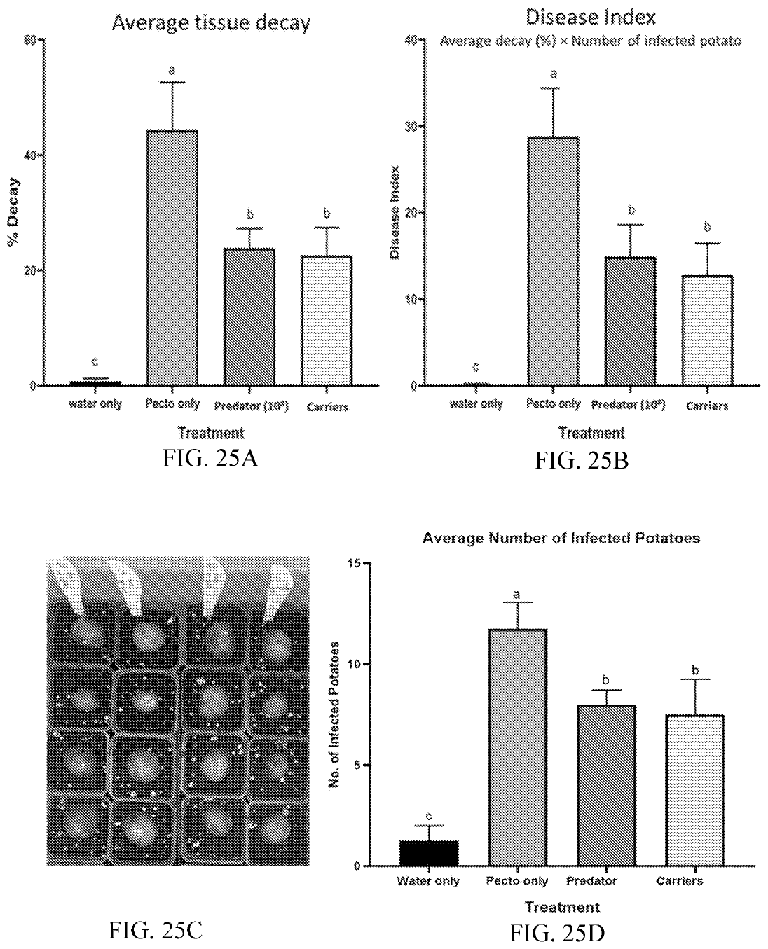

Protection by the κ-carrageenan hydrated formulation was similar to that obtained by the predator suspension, with significant lower averages (circa 50%) of tissue decay and disease index as compared to the control containing the pathogen alone (FIGS. 25A-25B).

### Example 22

### Protection of Potato Tubers Against Soft Rot by Predator Formulations Under Net-House Conditions

Potato tuber experiments were carried out in a net-house with controlled-irrigation in pots for four weeks until sprouting. Day/night conditions were natural conditions at ambient temperature, reaching 30° C. at times.

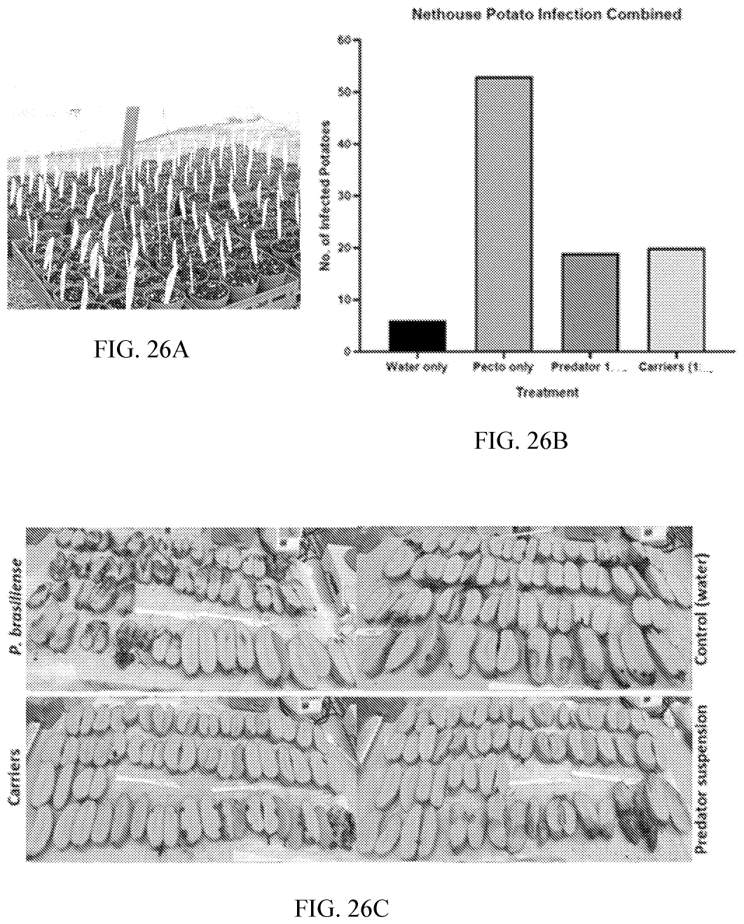

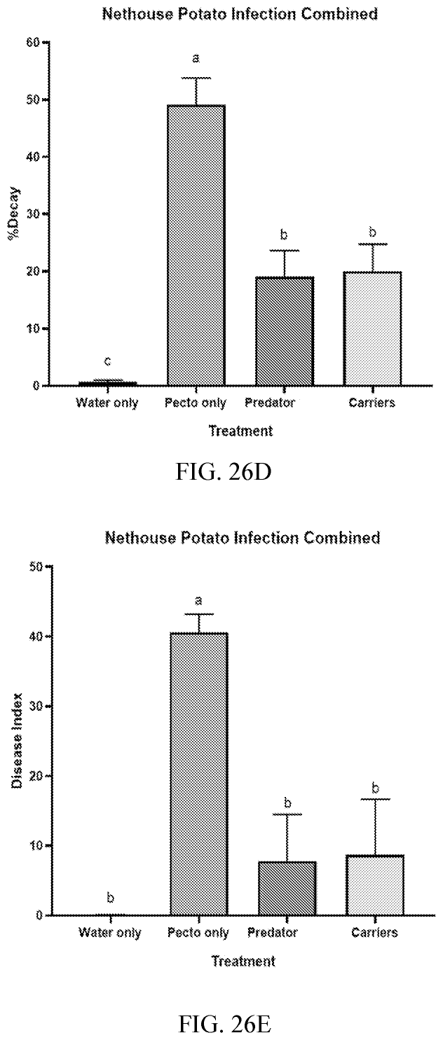

Both AP predator suspensions and dissolved carriers containing predators reduced the number of infected potatoes (FIG. 26B), the % decay (FIG. 26D) and the disease index (FIG. 26E) by at least ˜50%. Thus, encapsulation does not only keep the predators viable for long periods of time, it also does not alter their efficiency to provide protection against soft rot disease, even under natural conditions.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

### REFERENCE LIST

Aharon, E., Mookherjee, A., Pérez-Montaño, F., da Silva, G. M., Sathyamoorthy, R., Burdman, S., & Jurkevitch, E. (2021). Secretion systems play a critical role in resistance to predation by Bdellovibrio bacteriovorus. Research in microbiology, 172 (7-8), 103878.
Cao, H., Wang, H., Yu, J., An, J., & Chen, J. (2019). Encapsulated Bdellovibrio powder as a potential bio-disinfectant against whiteleg shrimp-pathogenic Vibrios. Microorganisms, 7(8), 244.
Duncan, M. C., Forbes, J. C., Nguyen, Y., Shull, L. M., Gillette, R. K., Lazinski, D. W., Ali, A., Shanks, R. M. Q., Kadouri, D. E., & Camilli, A. (2018). Vibrio cholerae motility exerts drag force to impede attack by the bacterial predator Bdellovibrio bacteriovorus. Nature communications, 9 (1), 1-9.
Fan, C., Guo, M., Liang, Y., Dong, H., Ding, G., Zhang, W., Tang, G., Yang, J., Kong, D., & Cao, Y. (2017). Pectin-conjugated silica microcapsules as dual-responsive carriers for increasing the stability and antimicrobial efficacy of kasugamycin. Carbohydrate polymers, 172, 322-331.
González, E., Herencias, C., & Prieto, M. A. (2020). A polyhydroxyalkanoate-based encapsulating strategy for ‘bioplasticizing’ microorganisms. Microbial Biotechnology, 13 (1), 185-198.
Jung, J., Arnold, R. D., & Wicker, L. (2013). Pectin and charge modified pectin hydrogel beads as a colon-targeted drug delivery carrier. Colloids and Surfaces B: Biointerfaces, 104, 116-121.
Liu, Y., Sun, Y., Ding, G., Geng, Q., Zhu, J., Guo, M., Duan, Y., Wang, B., & Cao, Y. (2015). Synthesis, characterization, and application of microbe-triggered controlled-release kasugamycin-pectin conjugate. Journal of agricultural and food chemistry, 63 (17), 4263-4268.
Mansfield, J., Genin, S., Magori, S., Citovsky, V., Sriariyanum, M., Ronald, et al. (2012). Top 10 plant pathogenic bacteria in molecular plant pathology. Molecular plant pathology, 13 (6), 614-629.
Necas, J., & Bartosikova, L. (2013). Carrageenan: a review. Veterinarni medicina, 58 (6).
Nussinovitch, A., & Hirashima, M. (2013). Cooking innovations. Boca Raton, FL: CRC Press (Chapter 4).
Rotem, O., Pasternak, Z., & Jurkevitch, E. (2014). Bdellovibrio and Like Organisms. In the Prokaryotes: Deltaproteobacteria and Epsilonproteobacteria. Rosenberg, E., DeLong, E. F., Lory, S., Stackebrandt, E., and Thompson, F. (eds). Berlin, Heidelberg: Springer Berlin Heidelberg, pp. 3-17.
Shemesh, Y., & Jurkevitch, E. (2004). Plastic phenotypic resistance to predation by Bdellovibrio and like organisms in bacterial prey. Environmental Microbiology, 6 (1), 12-18.
Sockett, R. E. (2009). Predatory lifestyle of Bdellovibrio bacteriovorus. Annual review of microbiology, 63, 523-539.
Thrimawithana, T. R., Young, S., Dunstan, D. E., & Alany, R. G. (2010). Texture and rheological characterization of kappa and iota carrageenan in the presence of counter ions. Carbohydrate Polymers, 82 (1), 69-77.
Vemmer, M., & Patel, A. V. (2013). Review of encapsulation methods suitable for microbial biological control agents. Biological Control, 67 (3), 380-389.

1.-50. (canceled)

51. A system for differential delivery of viable Bdellovibrio-and-like organisms (BALOs) comprising:
a carrier comprising polysaccharide hydrocolloid beads in a wet or a dried-gel form; and
BALOs immobilized within the carrier, wherein the BALOs are in a physiological state selected from the group consisting of bdelloplasts, bdellocysts, and attack phase cells (APs), which are present at a cell density of at least about 1.0×lO⁸ PFU/(g carriers),
wherein the system is in a form selected from: wet gel, dried-gel, or de-wetted gel, wherein the carrier preserves viability and bacteriolytic efficiency of the immobilized BALOs in the wet or the dried-gel form,
wherein the polysaccharide hydrocolloid is present in the system in a weight percent ranging from about 2% to about 25% out of the total weight of the dried-gel system, and
wherein said system comprises from about 0.1% (w/w) to about 10% (w/w) water when the system is in the dried-gel form.

52. The system according to claim 51, wherein the polysaccharide hydrocolloid is selected from the group consisting of κ-carrageenan, pectin, alginate, agar, gellan, carboxymethyl cellulose (CMC), furecellaran, xanthan gum, locust bean gum (LBG), konjac-mannan, chitosan, pullulan, curdlan and derivatives and combinations thereof.

53. The system according to claim 52, wherein the polysaccharide hydrocolloid is κ-carrageenan or low methoxyl pectin (LMP).

54. The system according to claim 51, wherein the polysaccharide hydrocolloid is crosslinked by a crosslinking agent and/or a gelation-inducing agent comprising a metal cation selected from the group consisting of a potassium (K⁺), calcium (Ca²⁺), barium (Ba²⁺), magnesium (Mg²⁺), aluminum (Al³⁺), zinc (Zn²⁺), lead (Pb²⁺), ferrous (Fe²⁺), strontium (Sr²⁺), copper (Cu²⁺), cadmium (Cd²⁺), nickel (Ni²⁺), cobalt (Co²⁺), and combinations thereof.

55. The system according to claim 51, wherein the carrier further comprises a protein hydrocolloid and/or a protein.

56. The system according to claim 55, wherein the protein hydrocolloid is gelatin present in the system in a weight percent ranging from about 20% to about 85% out of the total weight of the dry system.

57. The system according to claim 51, wherein the carrier further comprises at least one osmoprotectant in a weight percent ranging from about 20% to about 85% out of the total weight of the dry system.

58. The system according to claim 57, wherein the at least one osmoprotectant is selected from the group consisting of trehalose, inositol, glycerol, arabitol, betaine, an amino acid, and combinations thereof.

59. The system according to claim 58, wherein the at least one osmoprotectant is trehalose.

60. The system according to claim 51, wherein the differential delivery of the BALOs is characterized by a fast release of the BALOs from the carrier, wherein said carrier comprises κ-carrageenan.

61. The system according to claim 51, wherein the differential delivery of the BALOs is characterized by a suspended release of the BALOs from the carrier, wherein said carrier comprises low methoxyl pectin (LMP).

62. A method of preventing, reducing or eliminating a bacterial load in an item, a biofilm a plant, a plant part, soil, or a crop pre- or post-harvest, the method comprising contacting the item, the biofilm, the plant, the plant part, soil, or the crop pre- or post-harvest with an effective amount of the delivery system according to claim 1, thereby preventing, reducing or eliminating the bacterial load in said item, biofilm, plant, plant part, soil, or crop.

63. The method according to claim 62, wherein the bacteria of the bacterial load are phytopathogenic bacteria selected from the group consisting of P ectobacterium carotovorum, P ectobacterium chrysanthemi, P ectobacterium aroidearim, P ectobacterium atrosepticum, P ectobacterium betavasculorum, P ectobacterium cacticida, P ectobacterium colocasium, P ectobacterium cypripedii, P ectobacterium melonis, P ectobacterium parmentieri, P ectobacterium Polaris, P ectobacterium punjabense, P ectobacterium rhapontici, P ectobacterium versatile, P ectobacterium wasabiae, P ectobacterium zantedeschiae, Xanthomonas campestris, Pseudomonas syringae, Pseudomonas fluorescens, Pseudomonas tolaasii, Pseudomonas glycinea, Erwinia amylovora, Ralstonia solanacearum, Acidovorax citrulli, Acidovorax oryzae, Acidovorax avenae, Dickeya solani, Dickeya chrysanthemi, Dickeya dadantii, Xylella fastidiosa, and Pantoea sp.

64. The method according to claim 62, wherein the bacteria of the bacterial load are phytopathogenic and wherein the crop is selected from the group consisting of potato (Solanum tuberosum), onion (dry bulb and green), welsh onion (Allium fistulosum), shallot, carrot, soybean, barley, buckwheat, millet, oats, rice, rye, quinoa, sugar beet, teff, teosinte, triticale, wheat, wild rice, corn, soybean, cotton, borage, buffalo gourd (Cucurbila foetidissimd), canola, crambe, flax, jojoba, lesquerella, oilseed rape, safflower, sesame, sunflower, grain sorghum (milo), sugar cane, aloe vera, asparagus, bamboo shoots, globe artichoke, okra, peanut, pineapple, strawberry, allspice, angelica, star anise, annatto (seed), balm, basil, bumet, chamomile, caper buds, caraway, black caraway, cardamom, cassia bark, cassia buds, catnip, celery seed, chervil (dried), chive, Chinese chive, cinnamon tree, clary, clove buds, coriander leaf (cilantro or Chinese parsley), coriander seed (cilantro), costmary, culantro (leaf), culantro (seed), cumin, curry (leaf), dill (dillweed), dill (seed), epazote, fennel seed, fenugreek, white ginger flower, grains of paradise (Aframomum melegueta), horehound (Marrubium vulgare), hyssop, juniper berry, lavender, lemongrass, lovage (leaf and seed), mace, marigold, marjoram (including oregano), mexican oregano, mioga flower, mustard (seed), nasturtium, nutmeg, parsley, pennyroyal, pepper (black and white), pepper leaves, peppermint, perilla, poppy (seed), rosemary, rue, saffron, sage, savory (summer and winter), spearmint, stevia leaves, sweet bay, tansy, tarragon, thyme, vanilla, wintergreen, woodruff, wormwood, broccoli, Chinese broccoli (Gai Lan), broccoli raab (rapini), brussels sprouts, cabbage, Chinese cabbage (bok choy), Chinese cabbage (napa), Chinese mustard cabbage (Gai Choy), cauliflower, cavalo broccolo, collards, kale, kohlrabi, mizuna, mustard greens, mustard spinach, rape greens, garlic, great-headed garlic, leek, chayote (fruit), Chinese waxgourd (Chinese preserving melon), citron melon, cucumber, gherkin, hyotan, cucuzza, hechima, Chinese okra, melon, balsam apple, balsam pear, bittermelon, Chinese cucumber, cantaloupe, casaba, crenshaw melon, golden pershaw melon, honeydew melon, honey ball melon, mango melon, Persian melon, pineapple melon, Santa Claus melon, snake melon, pumpkin, crookneck squash, scallop squash, straightneck squash, vegetable marrow, zucchini, butternut squash, calabaza, hubbard squash, acorn squash, spaghetti squash, watermelon, amaranth (Chinese spinach), arugula (roquette), beet greens, cardoon, celery, Chinese celery, celtuce, chaya, chervil, edibleleaved chrysanthemum, garland chrysanthemum, cress (garden and upland), dandelion, dock (sorrel), dokudami, endive (escarole), Florence fennel, gow kee, lettuce (head and leaf), orach, parsley, purslane (garden and winter), dadicchio (red chicory), rhubarb, spinach, New Zealand spinach, vine spinach, Swiss chard, watercress (upland), water spinach, eggplant, groundcherry (Physalis spp), bell pepper, chili pepper, pepper, pimento, sweet pepper, tomatillo, tomato, legume vegetables, grain lupin, sweet lupin, white lupin, field bean, kidney bean, lima bean, navy bean, pinto bean, runner bean, snap bean, tepary bean, wax bean, adzuki bean, asparagus bean, blackeyed pea, catjang, Chinese longbean, cowpea, crowder pea, moth bean, mung bean, rice bean, southern pea, urd bean, yardlong bean, broad bean (fava), chickpea (garbanzo), guar, jackbean, lablab bean, lentil, dwarf pea, edible-podded pea, English pea, field pea, garden pea, green pea, snowpea, sugar snap pea, pigeon pea, soybean (immature seed), sword bean, arracacha, arrowroot, Chinese artichoke, Jerusalem artichoke, beet (garden), burdock, canna, cassava (bitter and sweet), celeriac, Chayote (root), chervil (turnip-rooted), chicory, chufa (Cyperus esculentus), dasheen (taro), galangal, ginseng, horseradish, leren, kava (turnip-rooted), parsley (turnip-rooted), parsnip, radish, oriental radish, rutabaga, salsify, back salsify, Spanish salsify, skirret, sweet potato, tanier, turmeric, turnip, wasabi, yacon, yam bean, true yam, and any combinations thereof.

65. The method according to claim 62, wherein the bacteria of the bacterial load are phytopathogenic and wherein the plant or plant part is selected from the group consisting of a seed, seedling, plantlet, tuber, bulb, rhizome, tree, herb, shrub, creeper, vegetable, fruit, legume, mushroom, flower, and combinations thereof.

66. The method according to claim 62, wherein contacting the item, biofilm, plant, plant part, soil, or crop with the delivery system is performed by a process selected from the group consisting of a foliar application, soil application, application through irrigation systems, starter solutions, injection to soil, patches application, aerial application, spraying, spreading, dripping, dipping, washing, incubating, and any combination thereof.

67. The method according to claim 62, wherein the item is selected from the group consisting of machinery and equipment for manufacturing or storage of pharmaceuticals; medical devices or implants; machinery or equipment for food preparation and storage; tanks, pipes, filters, reservoirs, sinks, coolers; water irrigation systems; textile, plant nurseries, aquaculture facilities, soil treatments, boats, ship maintenance, water treatment facilities, sewage treatment facilities, and any part thereof.

68. A method for the preparation of a delivery system for the differential delivery of viable Bdellovibrio-and-like organisms (BALOs), the method comprising:
(i) admixing a suspension comprising BALOs with a hydrocolloid solution comprising a polysaccharide hydrocolloid to form a hydrocolloid composition comprising the BALOs, wherein the BALOs are in a physiological state selected from the group consisting of bdelloplasts, bdellocysts, and attack phase cells (APs); and
(ii) admixing the hydrocolloid composition comprising the BALOs of step (i) with a solution comprising a crosslinking agent to form a carrier comprising polysaccharide hydrocolloid beads in a wet gel form, such that the BALOs are immobilized within the polysaccharide hydrocolloid carrier at a cell density of at least about 1.0×lO⁸ PFU/(g carriers), thereby forming the delivery system in a wet gel form,
wherein the carrier preserves viability and bacteriolytic efficiency of the immobilized BALOs when in a form selected from a wet gel, a dried-gel, or a re-wetted gel,
wherein the polysaccharide hydrocolloid is in a weight percent ranging from about 2% to about 25% out of the total weight of the system when in the dried-gel form, and
wherein said system comprises from about 0.1% (w/w) to about 10% (w/w) water out of the total weight of the system when in the dried-gel form.

69. The method according to claim 68, further comprising a step of drying the delivery system of step (ii), thereby forming a dry delivery system.

70. The method according to claim 69, wherein drying the delivery system is performed by at least one technique selected from vacuum drying, air drying, desiccant drying, fluidized bed drying, spray-drying, sun drying, and freeze-drying.
